(19) United States
(12) Patent Application Publication
(10) Pub. No.: US 2021/0047373 A1
(43) Pub. Date: Feb. 18, 2021

(54) BETA BARREL POLYPEPTIDES AND METHODS FOR THEIR USE

(71) Applicant: UNIVERSITY OF WASHINGTON, Seattle, WA (US)

(72) Inventors: Jiayi DOU, Seattle, WA (US); Anastassia VOROBIEVA, Seattle, WA (US); Jason C. KLIMA, Seattle, WA (US); David BAKER, Seattle, WA (US)

(21) Appl. No.: 17/041,363

(22) PCT Filed: Apr. 4, 2019
(86) PCT No.: PCT/US2019/025743

§ 371 Date: Sep. 24, 2020

Related U.S. Application Data
(60) Provisional application No. 62/652,813, filed on Apr. 4, 2018.

(51) Int. Cl. C07K 14/00; G16B 15/20; G01N 21/80

(52) U.S. Cl. CPC C07K 14/00; G01N 21/80; G16B 15/20

(57) ABSTRACT

Disclosed herein are de novo designed beta barrel polypeptides, methods for designing such polypeptides, and methods for their use.

### CROSS REFERENCE

This application claims priority to U.S. Provisional Patent Application Ser. No. 62/652,813 filed Apr. 4, 2018, incorporated by reference herein in its entirety.

### STATEMENT OF GOVERNMENT RIGHTS

This invention was made with government support under Grant No. HDTRA1-11-1-0041 awarded by the Defense Threat Reduction Agency and Grant Nos. CHE-1332907 and Fellowship awarded by the National Science Foundation. The government has certain rights in the invention.

### BACKGROUND

Anti-parallel β-barrels are excellent scaffolds for ligand binding, as the base of the barrel can accommodate a hydrophobic core to provide overall stability, and the top of the barrel provides a recessed cavity for ligand binding (often flanked by loops which can contribute further binding affinity and selectivity). However, as noted above, β-sheet topologies are notoriously difficult to design from scratch, with no reported success to date.

### SUMMARY

In one aspect the disclosure provides non-naturally occurring beta barrel polypeptides comprising the formula X1-X2-X3-X4-X5-X6-X7-X8-X9-X10-X11-X12-X13-X14-X15-X16-X17-X18-X19-X20, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta sheet with an internal G residue;

X15 comprises a single polar amino acid;

X16 comprises a beta turn;

X17 comprises a beta strand;

X18 comprises a beta turn; and

X19 comprises a beta strand.

In various embodiments, Z1 is a hydrophobic amino acid and Z2 is a polar amino acid; Z1 is selected from the group consisting of L, A, and F; and/or Z2 is selected from the group consisting of T, K, N, and D. In various other embodiments, the X1 capping domain comprises an alpha helix, and/or X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO: 121) or RAAQLLP (SEQ ID NO: 134), wherein the highlighted residue is invariant.

In various further embodiments:
X2 comprises the amino acid sequence at least 50%, 55%, 60%, 65%, 70%, 75%, 80%, 85%, 90%, 95%, or 100% identical to the amino acid sequence G (T/K/N/D) WQZT(M/F)TN (SEQ ID NO: 122) wherein Z is any amino acid, or GTWQ(V/L/A/I) T(M/F)TN (SEQ ID NO: 135), wherein the highlighted residues are invariant;
X3 comprises the amino acid sequence (E/S)DG or EDG;
X4 comprises the amino acid sequence at least 50%, 55%, 60%, 65%, 70%, 75%, 80%, 85%, 90%, 95%, or 100% identical to the amino acid sequence QTSQGQMHFQP (SEQ ID NO: 123), wherein the highlighted residues are invariant;
X5 comprises a single polar amino acid selected from the group consisting of R, T, Q, N, K, E, D, S, or wherein X5 is R;
X6 comprises the amino acid sequence (T/S)PZ3, where Z3 is polar amino acid or Tyr; or wherein X6 is SPY;
X7 the amino acid sequence at least 50%, 55%, 60%, 65%, 70%, 75%, 80%, 85%, 90%, 95%, or 100% identical to the amino acid sequence T(L/A/M)D(I/V)(K/V)(A/S) GT(I/M) (SEQ ID NO: 124) or TMDIVAQGTI (SEQ ID NO: 136), wherein the highlighted residues are invariant; X8 comprises the amino acid sequence (S/A)DG or SDG; X9 comprises the amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO: 125) or RPIVGYGKATV (SEQ ID NO:137), wherein the highlighted residues are invariant;
X10 is selected from the group consisting of R, T, Q, N, K, E, D, or S; or X10 is K;
X11 comprises the amino acid sequence (S/T)(P/C)(polar or Y), or wherein X 11 is TPD;
X12 comprises the amino acid sequence at least 50%, 55%, 60%, 65%, 70%, 75%, 80%, 85%, 90%, 95%, or 100% identical to the amino acid sequence T(M/L/V)(D/H/Q/N)(V/A/L/I)(D/N/H/Q)(I/L/V) T(Y/W) (SEQ ID NO: 126) or TLDIDITY (SEQ ID NO:138);
X13 comprises the amino acid sequence (S/E)DG, or wherein X13 comprises the amino acid sequence at least 60%, 80%, or 100% identical to PSLGN (SEQ ID NO: 127);
X14 comprises the amino acid sequence at least 50%, 55%, 60%, 65%, 70%, 75%, 80%, 85%, 90%, 95%, or 100% identical to the amino acid sequence (K/M/I/L)(Q/K)(V/A/G)QGQ(V/I)T(M/L/Y) (SEQ ID NO: 128) or IKAQGQITM (SEQ ID NO: 139), wherein the highlighted residues are invariant;
X15 is selected from the group consisting of R, T, Q, N, K, E, D, or S, or wherein X15 is D; X16 comprises the amino acid sequence (S/T)P(D/T/Y);
X16 comprises the amino acid sequence SPT;
X17 comprises the amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/F/V/Y) (SEQ ID NO: 129) or QFKFDATT (SEQ ID NO: 140);
X19 comprises the amino acid sequence at least 50%, 55%, 60%, 65%, 70%, 75%, 80%, 85%, 90%, 95%, or 100% identical to the amino acid sequence [(S/K/N/H)](K/R/N/H)(V/L)TGT(L/I/M)QRQE (SEQ ID NO: 132) or RLTGTLQRQE (SEQ ID NO: 144), wherein residues in brackets are optional; and/or
X18 comprises the amino acid sequence selected from the group consisting of (S/E/N/A/Q)DG, SDG, K(G/Q/K/T)(A/D/E/N)(G/D/N)(N/G/D/Y/S) (SEQ ID NO: 130), KG(A/D/E)(G/D/N)(N/G/D/Y) (SEQ ID NO: 131), KGENDFHG (SEQ ID NO:141), KGADGWHG (SEQ ID NO: 142), and KGAGNFTG (SEQ ID NO: 143).

In another embodiment, the beta barrel polypeptide further comprises a functional domain. In one embodiment, the functional domain is present within X18. In another embodiment, the functional domain comprises a detectable moiety including but not limited to a fluorescent protein or other chromophore; and a detector polypeptide including but not limited to a pH-responsive polypeptide, an ion-binding polypeptide, a small-molecule binding peptide, a nucleic acid binding polypeptide, an inorganic or organic substrate-binding polypeptide.

In various further embodiments, the beta barrel polypeptide comprises the amino acid sequence at least 50%, 55%, 60%, 65%, 70%, 75%, 80%, 85%, 90%, 95%, or 100% identical to the amino acid sequence selected from SEQ ID NOS: 1-120. In various further embodiments, the beta barrel polypeptide comprises the amino acid sequence at least 50%, 55%, 60%, 65%, 70%, 75%, 80%, 85%, 90%, 95%, or 100% identical to the amino acid sequence selected from SEQ ID NOS: 24-32, 37-66, 69, 75-76, 88-90, 92, and 94.

In further embodiments, the beta barrel polypeptide comprises the amino acid sequence at least 50%, 55%, 60%, 65%, 70%, 75%, 80%, 85%, 90%, 95%, or 100% identical to the amino acid sequence of SEQ ID NO: 38 (mFAP2). In various further embodiments, the polypeptide comprises residues at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or all 45 of the following positions relative to SEQ ID NO: 38 (mFAP2), with numbering starting from the first residue after the optional N-terminal methionine residue:

| Position (mFAP2 numbering, no M) | Residues |
|---|---|
| 13 | V, A, L, I |
| 15 | M, F |
| 17 | N |
| 23 | S, T |
| 27 | W, M |
| 29 | F, I |
| 37 | M, L |
| 39 | I, V |
| 41 | A |
| 45 | I, M, L |
| 49 | R |
| 50 | P, T |
| 51 | I |
| 52 | V, S, Q, T |
| 57 | A, V, L |
| 59 | V, A, C |
| 65 | L, M, V |
| 67 | I, V, A |
| 69 | I, L |
| 71 | Y, W |
| 72-76 | PSLGN (SEQ ID |
| | NO: 127) |
| 77 | I, M, L |
| 79 | A, G, V |
| 83 | I, V |
| 85 | M, L, Y, N |
| 91 | F, A |
| 93 | W, F |
| 95 | A, G, S |
| 97 | T |
| 98-100 | KG (E/A) |
| 101-103 | (N/G/D) (D/N/G) F |
| 104-106 | (H/T/Q) GR |
| 105 | F, W, Y |
| 107 | L, V |
| 111 | L, I, M |

In various further aspects, the disclosure provides nucleic acids encoding the beta barrel polypeptide of any embodiment or combination of embodiments of the disclosure; expression vectors comprising the nucleic acids of the disclosure; recombinant host cells comprising the nucleic acids and/or the expression vectors of the disclosure, and pharmaceutical compositions, comprising a pharmaceutically acceptable carrier combined with the beta barrel polypeptides, nucleic acids, expression vectors, and/or the recombinant host cells of any embodiment or combination of embodiments of the disclosure.

In a further aspect the disclosure provides uses of the beta barrel polypeptides, nucleic acids, expression vectors, and/or the recombinant host cells and/or the pharmaceutical compositions of any embodiment or combination of embodiments of the disclosure, for uses including, but not limited to pH sensing, ion-sensing/detection (including but not limited to Ca²⁺, La³⁺, Tb³⁺, and other ion sensing/detection/quantification), super-resolution microscopy, localization microscopy, and detection and quantification of other small-molecules, ions, organic or inorganic substrates, peptides, or nucleic acids by insertion of their respective binding peptides into the loops or turns of the polypeptides.

In another aspect, the disclosure provides methods for designing beta barrel polypeptides, comprising any embodiment or combination of embodiments of polypeptide design steps disclosed herein.

### DESCRIPTION OF THE FIGURES

FIG. 1: Principles for designing β-barrels. Two methods for β-barrel generation. A. Parametric generation of 3D β-barrel backbones based on the hyperboloid model (Methods). The cavity geometry is controlled on the global level through the model parameters (rA and rB). B. 2D map-guided approach defining residues connectivity and subsequent assemble of 3D backbones with Rosetta. The cavity geometry is controlled on the local level by specification of backbone torsion angle bins for each residue. C. Both glycine kinks and bulges release Lennard Jones repulsive interactions in β-barrels. Full backbones are shown on the left and the Cβ-strip along the black arrow is shown on the right. (Top) No bulge, no glycine kink; (Middle) one glycine kink in the middle of each Cβ-strip, no bulge; (Bottom) one glycine kink in the middle of each Cβ-strip, bulges associated with each turn. D. Blueprint used to generate a β-barrel of type (N=8; S=10) with a square cavity for ligand binding. The overall radius r of the barrel and the tilt θ of the strands are determined by the choice of n and S as indicated. The residues in the 2D blueprint (left) and the 3D structure (middle) are colored by backbone torsion bin (right). Backbone hydrogen bonds in the 2D representation are shown as dashed lines. Shaded and open circles represent residues facing the barrel interior and exterior, respectively. “Strain-relieving” glycine positions are shown as yellow circles and bulges as stars. Vertical dotted lines show the axes along which the sheet is designed to bend.

FIG. 2: Folding, stability and structure of BB1. A. In silico folding energy landscape; each point is the result of an independent ab initio folding calculation (gray circles), a refinement trajectory starting from the lowest energy ab initio models (grey crosses) or starting from designed structure (black). B. SEC trace of the purified monomer. C. Far ultraviolet circular dichroism spectra at 25° C. (grey line), 95° C. (black dashed line) and cooled back at 25° C. (black dotted line). D. Near-UV CD spectra in Tris buffer (grey line) or GuHCl (black line). E. Cooperative unfolding in GuHCl followed by near-UV CD signal at 285 nm (grey line) and tryptophan fluorescence (black line). F-J: Superpositions of the crystal structure (grey) and the design model. F. Overall backbone superposition. G. Section along the β-barrel axis showing the rotameric states of core residues. H. One of the top loop with G1 β-bulge. I. Equatorial section of the β-barrel, showing the shape of the cavity. The glycine kinks are shown as sticks. The bottom of the panel shows the cross-section of the three closest native β-barrel structures based on TMscore (PDB IDs 1JMX (0.77); 41L6O (0.73); 1PBY (0.71)). J. One of the bottom loops with β-bulge. K. Sticks representation and experimental electron density of the designed tryptophan corner.

FIG. 3: Computational design and structural validation of β-barrels with recessed cavities for ligand binding. a, (Left) Ensembles of side chains generated by the RIF docking method making hydrogen bonding (upper left) and hydrophobic interactions (lower left) with DFHBI. (Right) Ensemble of 200 β-barrel backbones, with Cα atoms surrounding the binding cleft indicated by magenta spheres. b, Scaffold-ligand pairs (left) with multiple ligand-coordinating interactions from RIF docking are subjected to Rosetta energy-based sequence design calculations (right): positions around the ligand (above the dashed line) are optimized for ligand binding; the bottom of the barrel (dark grey, below dashed line), for protein stability. c, (Left) Crystal structure (cartoon with grey surface) of b10 with a recessed binding pocket filled with water molecules (spheres). (middle) b10 design model backbone superimposed on the crystal structure. (Right) Comparison of crystal structure and design model for two different barrel cross sections (indicated by dashed lines); glycine Cα atoms are indicated by spheres in the upper layer.

FIG. 4: Sequence dependence of fold and function. a, (Upper) b11L5F backbone model colored by average fluorescence activation scores. Blue positions are strongly conserved during yeast selection; red positions are frequently substituted by other amino acids. Residues buried in the design model are much more conserved than solvent exposed residues. (Lower) All the mutations to the Gly-kinks (spheres in the model) and tryptophan (W) corner reduced the fluorescence activity significantly, in consistency with a critical structural role for the designed function. b & c, Bottom (b) and top (c) comparisons of b11L5F side chains colored by average fluorescence activation scores (upper row) and stability scores (lower row). In the bottom barrel, core residues are strongly conserved in both function and stability selections (b); in the top barrel there is a clear function-stability tradeoff with the key DFHBI interacting residues critical for function but far from optimal for stability (c). Individual mutations for seven DFHBI-coordinating residues from RIF docking were shown as heat maps (c right).

FIG. 5: Structure and function of mFAPs. a & b, 2Fo-Fc omit electron density in the mFAP1-DFHBI complex crystal structure. DFHBI is clearly in the high energy cis-planar conformation (a) stabilized by closely interacting residues (b). The density map was contoured at 1.0σ. c, Superposition of mFAP1 design model and the crystal structure. Hydrogen bonds coordinating DFHBI are indicated by dashed lines. d, Fluorescence emission spectra of 0.5 μM DFHBI with or without 5 μM mFAPs, excited at 467 nm. e & f, Confocal micrographs of fluorescent E. coli cells expressing mFAP2 (e) and yeast cells displaying Aga2p-mFAP2 fusion proteins on the cell surface (f). g & h, Confocal micrographs of live mammalian cells expressing mFAP2 (g) and mFAP2 fusions to tags or proteins localized to the mitochondria outer membrane (Tom20, right), the mitochondria inner membrane (human cytochrome c oxidase targeting sequence, middle), and the endoplasmic reticulum (Sec61β, left) (h). DFHBI concentration was 20 μM for all the cells.

FIG. 6: Comparison of structures of GFP and mFAP1 a, Surface mesh and ribbon representations of structures of GFP (left, PDB ID: 1EMA) and the computationally designed mFAP1 (right) with the chromophores embedded in the protein (spheres). GFP, a product of natural evolution, has more than twice the number of residues, and a taller (top panel) and wider (bottom panel) barrel. Water molecules are shown as light spheres. b, Close up of chromophore binding interactions in GFP (left) and mFAP1 (right).

FIG. 7: Parametric design: workflow and shortcoming. A. Schematic representation of the parametric approach to generate beta-barrel backbones. (B. and C.) Comparison between beta-barrels of type (n=8; S=8, B) and type (n=8; S=10, C); showing the 2D map of residues connectivity (top), the arrangements of the CPs in the Cβ-strips (middle) and the packing pattern of the core side-chains (bottom). The difference in sheer number translates into different overall strand staggering and barrel radii, and a different number of core Cβ-strips (top, middle), which results in different packing arrangement with side-chains in the core of the barrel. (D-F) The final parametric designs exhibited distorted hydrogen bonds, based on deviation of C—O—H angles distribution of the backbone hydrogen bonds in the designs to native proteins (E), and the register shift between hydrogen bonded residues (F). These two metrics are defined in D.

FIG. 8: Glycine kinks and beta-bulges remove excessive strain in beta-barrel backbones. A. In the absence of local torsional irregularities, all arrangements of beta-strands generated either with the hyperboloid, cylindrical, coiled-coil parametric models or with Rosetta's fragment protocol and relaxed with valine at every position lose a significant fraction of the hydrogen bond interactions (white). More interactions are retained with one residue in the middle of each Cβ-strip mutated to glycine before relaxation (grey). B. The strain in the beta-barrel backbone due to sheet closure is translated as unfavourable local left-handed twist, which can be accommodated by glycine kinks. (Top) The local strand twist is calculated using a sliding window of 4 residues along each β-strand (the Cα of residues of one strand are shown here as spheres) and defined as the angle between the vectors Cα1-Cα3 and Cα2-Cα4. The handedness of the twist is defined as the triple scalar product between these two vectors and the barrel major axis (scattered arrow). Positive and negative values denote right-handed and left-handed twist, respectively. (Bottom) Local twist calculated along each beta-strand of fragments-generated backbones without and with glycine kinks (grey), relaxed with constraints to maintain hydrogen bonds between strands. C. After relaxation, the positions in the middle of each Cβ-strip remain in the beta-sheet B ABEGO space if they remain valine (top right) or are distributed partly to the positive phi space (E ABEGO) if mutated to glycine. D. Similar torsion angle distribution is observed for glycines in the β-strands of native β-barrels.

FIG. 9: Placement of beta-bulges and choice of beta-turns. A. Glycine kinks increase local β-strand curvature and create a “corner” in the β-sheet. Backbones relaxed with and without glycine kinks (shown as sticks) were colored according to the local beta-strand curvature. The curvature is defined in (B) as described in². Because of strand staggering pattern and loop chirality, different bulge positions and beta-turns were selected for the top and bottom hairpins. The bottom of the barrel was defined as the side of the- and C-termini. C. β-bulges were aligned with the corners to further promote curvature, at position +1 relative to the top beta-turns and position −2 relative to the bottom beta-turns. D. Analysis of the beta-turns of a set of 35 native beta-barrels. The canonical GG turn is under represented in the dataset. The most abundant turn is AAG—which has an intrinsic beta-bulge at position +1 (G bulge). The second most abundant β-turn is AA, which is usually less abundant in beta-sheet proteins because its internal torsion is less compatible with the β-strand twist. We found that the AA turn is favored in native proteins when it starts at position 2 relative to a beta-bulge (E). The AAG turn was used to connect the top hairpins and the AA turn was used to connect the bottom hairpins.

FIG. 10: Deviations of BB1 crystal structure from the design model. A. Crystal contacts are mainly mediated by residues from the β-turns. One of the three bottom turns in the crystal structure (grey) significantly deviates from the design model (magenta) and forms additional crystal contacts. B. Top: Three phenylalanine have different rotameric states in the crystal structure (grey) and the design model. In the crystal structure, Phe41 interacts with Gly53 (aromatic rescue interaction³²), which shows the most backbone deviation between the crystal structure and the design (bottom). C. We ran MD simulation on the crystal structure and found that the most stable Phe41 rotamer across the MD trajectory was the rotamer in the design model. The lowest energy rotamer after minimization and repacking the MD refined model with Rosetta was also different from the rotamer characteristic of the aromatic rescue interaction. It is likely that the different phenylalanine rotamers results from the difficulty to accurately capture the long-range aromatic rescue interaction.

FIG. 11: Scaffold construction and post-design ligand docking simulations. a, Three geometric constraints used for constructing beta barrel scaffolds in the low-resolution centroid modeling stage. b, Ramachandran plots for beta barrel scaffolds. Backbones generated with all three constraints (a) had a very narrow phi/psi distribution (Set 1, upper left); by dropping N—H—O angle constraint (a), backbone torsion diversity gained slight improvement (Set 2, upper right). Both sets showed broadened phi/psi distribution after two rounds of sequence design calculation using Rosetta full-atom force field (lower panel). c, Total numbers of unique DFHBI-scaffold pairing solutions from RIF docking for each set of scaffolds. Backbones with better torsional diversity seems to yield more unique RIF docking solutions. d, Computed metrics for 42 designs ordered and tested. Results from ab initio folding simulation were scaled to 0.0 to 1.0, with 1.0 represents a funnel-shaped folding landscape³³. e, Alternative ligand binding conformations revealed by post-design ligand docking simulations. With rigid protein sidechain, the designed DFHBI-binding mode was close to the lowest-energy docking conformation (left, designed binding mode was circled in grey in the energy landscape). Ligand docking simulations using the apo protein model after extensive MD-based structure refinement found an alternative pseudo-symmetric docking conformation indicated by a circle in the docking energy landscapes. Three designed hydrogen bonding residues remain the same in two conformations (upper panel).

FIG. 12: Biochemical and structural characterization of design b10. a, Size-exclusion chromatogram of His6-tagged b10 after Ni-NTA affinity purification. The elution volume corresponds to a monomeric protein of approximate 11 kDa. b, Far-UV circular dichroism (CD) characterization of b10. Spectra at different temperatures (left) showed b10 refolds after one heating-cooling cycle. Thermal melting curve(right) was collected by monitoring the CD signal at 220 nm. c, Designed ligand binding pocket in b10 crystal structure containing multiple water molecules. d, DFHBI binding interactions in the b10 design model. e, Superposition of binding sites in b10 design model and crystal structure. Sidechain differences are highlighted by dashed circles.

FIG. 13: Experimental characterization of design b11. a, Size-exclusion chromatogram of His6-tagged b11 (solid line) and b38 (dashed line) after Ni-NTA affinity purification. b38 elutes before 12.5 mL indicating the formation of bigger oligomers, while b11 contains a small monomer fraction that elutes around 15 mL. b, Mutations mapped onto b11 and b38 design models. A disulfide bond connecting the N-terminal helix to the beta strand (Q1C and M59C) were introduced into b38 as a design strategy to help monomeric protein folding. c, Far-UV CD characterization of b11. b11 forms an amyloid-like beta structure at 95 degrees Celsius with a negative peak around 226 nm³⁴ and refolds back after cooling to 25 degrees Celsius. Thermal melting curves monitored at 226 nm in the presence of 1 mM tris(2-carboxyethyl) phosphine (TCEP) showed decreased thermal stability. d, Purified b11 protein activating DFHBI fluorescence. With 10 μM DFHBI, b11 showed concentration-dependent fluorescence activity. e, Designed interacting residues contributing to b11-DFHBI fluorescence. Single or double knockouts of hydrogen bonding residues (Y71, S23, N17 and T95) and a hydrophobic packing residue (M15) showed decreased fluorescence intensity at 500 nm in comparison with the wild-type b11 (WT).

FIG. 14: Experimental and in silico characterization of design b11L5F. a & b, Backbone conformations and sequences of the original fifth turn in b11 (a) and the redesigned 5-residue fifth turn in b11L5F (b). Backbone hydrogen bonds are indicated by dashed lines. c, Yeast cells displaying b11 or b11L5F incubated with or without 500 μM DFHBI were analyzed by flow cytometry. d, Purified b11L5F protein improved the fluorescence activity by 5.2 fold in comparison with b11 (100 μM protein+10 μM DFHBI). e, Ligand-docking simulations with refined apo model of b11L5F. Energy landscape was plotted by comparing all the docking conformations to the lowest-energy conformation, which is consistent with lowest-energy docking conformation for b11 (transparent grey, also see Extended Data FIG. 3e). The second lowest-energy ligand conformation was in the same orientation.

FIG. 15: Incorporation of point mutants improving fluorescence activation without compromising protein stability. a & b, Beneficial mutations at three positions mapped onto the b11L5F design model (a) and their corresponding scores (b). c, Fluorescence activation was improved with single and double mutations (left). Binding titration curves were obtained for all six triple mutations (right). d, Disulfide-bonding cysteines were mutated to Valine and Serine in addition to the triple mutant 83I_95A_103L and resulted “b11L5F.1”. e, Far-UV CD characterization of b11L5F.1. Thermal melting curves was monitored at 226 nm.

FIG. 16: Crystal structure of b11L5F_LGL (Protein samples of all six triple mutants in FIG. 15c were prepared for crystallization. b11L5F_LGL with 83L/95G/103L combination was successfully crystallized). a, Crystal contacts. Contacts between proteins in one asymmetric unit were mediated by two tyrosines in stick representation (grey dashed circle); contacts between three asymmetric units were formed between β-turns (black dashed circle). b, Backbone superposition of design model and crystal structure showed a loop displacement possibly due to the crystal contacts. c, Disulfide bond in the crystal structure matched to the design model. d, Side chain conformations in the design models matched the crystal structure in most cases. Backbone Cα RMSD upon alignment is 1.02 Å. e, Ligand density in the crystal structure. Left: 2Fo-Fc omit map showing the electron density after refinement without placing DFHBI. Middle: best ligand placement to match the density. Right: designed ligand binding interactions (silver) overlaid with the crystallized binding pocket.

FIG. 17: Characterization of five designs from the second round of design calculation. a, Binding titrations curves for b11L5F.1 and five new designs. b11L5F.2 showed the best fluorescence activity. b, 13 mutations introduced in b11L5F.2 were mapped onto the design model. c, Far-UV CD characterization of b11L5F.2. Thermal melting curves monitored at 226 nm. d, Ligand-docking simulations with the refined apo model of b11L5F.2. (Left) Energy landscape was plotted by comparing all the docking conformations to the design model. (Right) Lowest-energy docking conformations matches the design model.

FIG. 18: Characterization of three best variants from library selection. a, Yeast cells displaying mFAP proteins incubated with 5 μM DFHBI analyzed by flow cytometry. b, Fluorescence activity of purified proteins incubated with 0.5 μM DFHBI (excitation: 450 nm; emission: 500 nm and 510 nm). c & d, Mutations in mFAPs mapped on the design models (c) and their corresponding far-UV CD characterization (d). Common mutations in all three mFAPs were highlighted in bold. Thermal melting curves were recorded at 226 nm.

FIG. 19: Crystal structures of mFAP0 and mFAP1 in complex with DFHBI. a & b, Crystal contacts in the DFHBI-bound structures of mFAP0(a) and mFAP1(b). Contacts between proteins in one asymmetric unit were formed around 40V and 54Y (dashed circles) that were introduced for helping crystallization. Contacts between asymmetric units were formed between β-turns (black dashed circle). c, 2Fo-Fc omit electron density of DFHBI in the mFAP0-DFHBI complex crystal structure. DFHBI is in the higher energy cis-planar conformation. The density map was contoured at 1.0σ. d, Superposition of mFAP0 design model (silver) and the crystal structure (magenta). Hydrogen bonds were indicated by dashed lines. e, Helical capping interactions mediated by P62D mutation in mFAP1.

FIG. 20: Characterization of brighter and chromophore-specific mFAP variants (mFAP2a and mFAP2b). (a) Design model of de novo β-barrel variant mFAP2b protein backbone (cartoon) bound to small molecule chromophore DFHBI (sticks). (b, c) Chemical structures of the chromophores DFHBI and DFHBI-1T, respectively. (d, e) In vitro titration of (d) DFHBI or (e) DFHBI-1T with purified mFAP2, mFAP2b, and mFAP2a proteins. mFAP2a/DFHBI, mFAP2a/DFHBI-1T, and mFAP2b/DFHBI complexes are brighter than the mFAP2/DFHBI complex. mFAP2a has higher affinity toward DFHBI compared to mFAP2b. mFAP2a binds DFHBI-1T with higher affinity than mFAP2 and the complex has brighter fluorescence than the DFHBI systems. Error bars represent standard deviation of the mean of 8 technical replicates. Shown are the non-linear least squares fits to a single binding-site isotherm equation (lines). (f-i) Each panel shows an image of the fluorescent signal emitted by E. coli cells expressing the indicated mFAP variant labeled with 10 μM of the indicated chromophore (left) and a zoom on the modeled binding pocket of that mFAP variant bound to the chromophore (right). (f) mFAP2b with DFHBI, (g) mFAP2b with DFHBI-1T does not give a fluorescent signal, presumably because binding of DFHBI-1T into the mFAP2b binding pocket is inhibited by steric clashes with V13 (shown as cylinders and circled), (h) mFAP2a with DFHBI, (i) mFAP2a with DFHBI-1T. (f-i) The images on the left of each panel were acquired using a laser scanning confocal fluorescence microscope and show pseudo-colored normalized fluorescence intensity values per pixel. Scale bars represent 10 microns. In the design models shown on the right of each panel the residues unique to mFAP2b (V13, M15) and residues unique to mFAP2a (A13, F15) are shown as sticks. Intermolecular hydrogen bonds to the chromophore are shown as black dotted lines. Vacuum electrostatic contact potential around chromophore shown in a transparent grey surface.

FIG. 21: Super-resolution microscopy and photostability of mFAP2a and mFAP2b. (a-g) To show the application of the mFAP system in super-resolution microscopy, mFAP2a and mFAP2b were fused to a designed protein polymerizing into a filament (DHF119). (a) The maximum diameter of the DHF119 fiber fused to the 6×His-tagged mFAP2b was estimated to be ˜22 nm based on the computational design models. (b-d) The DHF119-GS-mFAP2a-6×His fiber was labeled at 5.78 μM DFHBI-1T and (e-f) the DHF119-GS-mFAP2b-6×His was labeled at 1.83 μM DFHBI. Both systems were imaged with (b,e) fluorescence widefield microscopy and (c,f) fluorescence localization microscopy. Scale bars are 1 micron. (d,g) Normalized average intensity profiles between the yellow arrowheads shown in the widefield image (black line) or localization reconstruction (grey line) estimate the diameter of the fluorescent filament. The mean and standard deviation of the mean of the full width half maximum (FWHM) values of the three intensity profiles are annotated, 11-k) Photostability of mFAP2a and mFAP2b complexes with DFHBI and DFHBI-1T compared to AcGFP1. A higher fluorescent signal is retained in fixed COS-7 cells expressing mFAP2a in the endoplasmic reticulum (h) between frame 1 (left) and frame 200 (right) when labeled with 50 μM DFHBI, compared to fixed COS-7 cells expressing AcGFP1 in the endoplasmic reticulum (i). Normalized fluorescence intensity images are shown for four regions of interest (ROIs). (h, i) Frames 1 (left) to 200 (right) were acquired under continuous wave imaging at 0.885 Hz (1.13 s/frame). Scale bars are 10 microns. (j, k) Average of the normalized image intensities from four ROIs under continuous wave imaging at 0.885 Hz (1.13 s/frame) for (j) mFAP2a labeled at 50 μM DFHBI, mFAP2a labeled at 0.5 μM DFHBI, mFAP2a labeled at 50 μM DFHBI-1T, mFAP2a labeled at 0.5 μM DFHBI-1T, AcGFP1, and (k) mFAP2b labeled at 50 μM DFHBI, mFAP2b labeled at 0.5 μM DFHBI, AcGFP1. (j, k) Standard deviations of the average normalized image intensities of four ROIs are shown as shading.

FIG. 22: In vitro characterization of the pH-responsive mFAP_pH and comparison to the existing pHRed system and pH-unresponsive mFAP2b. (a-c) Chemical basis of pH-responsiveness in mFAP_pH. (a) Chemical structures of protonated tautomers and deprotonated resonance structures of DFHBI. The protonated (phenolic) and deprotonated (phenolate) forms of DFHBI exhibit different fluorescence properties. The arrangement of intermolecular hydrogen bonds in the binding site of mFAP2b (c, binding pocket of the design model with the phenolate form of DFHBI) has low shape complementarity to the protonated DFHBI tautomers. The mutations W27M and W93F improve binding to the protonated DFHBI tautomers (b, model of mFAP_pH binding pocket with the phenolic form of DFHBI). In the panels (b) and (c) the residues unique to mFAP_pH (M27, F93) and the residues unique to mFAP2b (W27, W93) are shown as sticks. (d) Normalized fluorescence excitation spectra of the mFAP_pH/DFHBI complex are shown for a pH titration from pH 3.63 to pH 8.38. (e) mFAP_pH normalized fluorescence emission spectra at pH 3.63 and pH 8.38. (f, g) Comparison of the excitation spectra of the pH-responsive mFAP_pH (f) and the pH-unresponsive mFAP2b (g) at pH 3.61 and pH 7.34. (f) Binding of mFAP_pH to both the phenolic and phenolate forms of DFHBI results in a high fluorescence ratio fold-change from low to high pH. (h, i) pH-dependent fluorescence of the mFAPpH/DFHBI complex compared to the previously described pHRed system. (h) Normalized fluorescence measurements from the pH titration of mFAP_pH (dark grey lines) and pHRed (light grey lines) where: (dark grey, from top left to bottom right) fluorescence emission from the protonated DFHBI tautomers exciting the blue-shifted fluorescence excitation peak (λₑₓ=379 nm and λₑₘ=498 nm); (dark grey, from bottom left to top right) fluorescence emission from the deprotonated DFHBI resonance structures exciting from red-shifted fluorescence excitation peak (λₑₓ=483 nm and λₑₘ=503 nm); (light grey, from top left to bottom right) fluorescence emission from the blue-shifted fluorescence excitation peak (λₑₓ=440 nm and λₑₘ=635 nm); and (light grey, from bottom left to top right) fluorescence emission from the red-shifted fluorescence excitation peak (λₑₓ=575 nm and λₑₘ=635 nm). Error bars represent the standard deviation of the mean of 3 technical replicates. The means are fit to either a sigmoid equation or an inverse sigmoid equation using non-linear least squares fitting. (i) Fluorescence ratios (Fᵣₐₜᵢₒ) of unnormalized fluorescence measurements from panel (h) for mFAP_pH and pHRed. In panels (h) and (i), the dotted lines indicate pH values at which the measured fluorescence ratio (Fᵣₐₜᵢₒ in panel (i)) coincides with two different pH values, and therefore is not used in the non-linear least squares fitting. This anomalous behavior of pHRed at low pH is likely due to the denaturation of the protein. By comparison to pHRed, the mFAP_pH system is stable at low pH and exhibited a more broad dynamic range of pH sensing. (i) For mFAP_pH and pHRed, Fᵣₐₜᵢₒ is calculated from the unnormalized fluorescence measurements from panel (h) using fluorescence emission from the red-shifted excitation peak as the numerator and fluorescence emission from the blue-shifted excitation peak as the denominator. Error bars represent the standard deviation of the mean of 3 technical replicates. The means are fit to continuous logistic equations with the formulae

$${F}_{ratio} = \frac{23.48}{1 + {e}^{- 2.02 \cdot ( pH - 6.90 )}}$$

for mFAP_pH and

$${F}_{ratio} = \frac{4.20}{1 + {e}^{2.00 \cdot ( pH - 6.60 )}}$$

for pHRed.

FIG. 23. In vitro fluorescence characterization of Ca²⁺-responsive mFAP variants. Different constructs combining a variable number of EF-hand motif insertions into the loop7 of mFAP2b or mFAP2a with different linker sequences produced peptides exhibiting positive or negative allostery between Ca²⁺ and DFHBI binding. (a-c) DFHBI titration in the absence of Ca²⁺ (squares) and presence of Ca²⁺ (circles) (a) mFAP2b demonstrating no allostery between Ca²⁺ and DFHBI binding, (b) EF1p2_mFAP2b demonstrating positive allosteric modulation, and (c) EF2n_mFAP2b demonstrating negative allosteric modulation. Normalized fluorescence values (n=1) are fit to a sigmoid equation using non-linear least squares fitting (lines). (d-f) Ca²⁺ titration with excess DFHBI concentrations compared to protein concentrations show that the dynamic range of calcium sensing is dependent on the number of EF-hand motifs inserted into loop7 (d) mFAP2b demonstrating a lack of Ca²⁺-responsiveness, (e) EF1p2_mFAP2b (containing one EF-hand motif on loop7) showing normalized fluorescence and demonstrating positive allostery between DFHBI and Ca²⁺ binding, and (f) from right to left: EF1n_mFAP2b (containing one EF-hand motif on loop7, K_{d}=260 EF2n_mFAP2b (containing two EF-hand motifs on loop7, K_{d}=60 and EF4n_mFAP2b (containing four EF-hand motifs on loop7, K_{d}=7 μM) showing normalized fluorescence and demonstrating negative allostery between DFHBI and Ca²⁺ binding. Error bars represent the standard deviation of the mean of 3 technical replicates. The means are fit to a sigmoid equation (for positive allostery) or inverse sigmoid equation (for negative allostery) with Hill coefficients of 1 using non-linear least squares fitting (lines) to extract reported K_{d} values in Table 5.

FIG. 24A-C. Amino acid sequence alignment of exemplary polypeptides of the disclosure.

FIG. 25A-B. Amino acid sequence alignment of exemplary polypeptides of the disclosure.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect the disclosure provides non-naturally occurring polypeptides comprising the formula X1-X2-X3-X4-X5-X6-X7-X8-X9-X10-X11-X12-X13-X14-X15-X16-X17-X18-X19-X20, wherein:

X1 comprises a capping domain;

X2 comprises a beta strand,

wherein a contiguous C-terminal portion of X1 and N-terminal portion of X2 comprise the amino acid sequence Z1-P-G-Z2-W, where Z1 and Z2 are any amino acid;

X3 comprises a beta turn;

X4 comprises a beta strand that includes an internal G residue and a P at its C terminus;

X5 comprises a single polar amino acid;

X6 comprises a beta turn;

X7 comprises a beta strand including an internal G residue;

X8 comprises a beta turn;

X9 comprises a beta strand including an internal P residue and 2 internal G residues;

X10 comprises a single polar amino acid;

X11 comprises a beta turn;

X12 comprises a beta strand;

X13 comprises a beta turn;

X14 comprises a beta sheet with an internal G residue;

X15 comprises a single polar amino acid;

X16 comprises a beta turn;

X17 comprises a beta strand;

X18 comprises a beta turn; and

X19 comprises a beta strand.

As demonstrated in the examples that follow, the polypeptides disclosed herein constitute the first successful de novo design of a β-barrel polypeptide, and the first de novo design of the fold and function of a small molecule binding protein.

As used herein, a “capping domain” is any sequence of amino acids that appropriately position the Z1-P-G-Z2-W domain noted above (also referred to herein as the “tryptophan corner’). As such, the capping domain may be of any suitable length and amino acid composition. In one non-limiting embodiment, the capping domain may comprise an alpha helical domain. Exemplary capping domains are provided in the specific polypeptide sequences disclosed herein.

In one embodiment, Z1 is a hydrophobic amino acid and Z2 is a polar amino acid. In another embodiment, Z1 is selected from the group consisting of L, A, and F, or Z1 is L. In a further embodiment, Z2 is selected from the group consisting of T, K, N, and D, or Z2 is T. In one embodiment, X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO: 121) or RAAQLLP (SEQ ID NO: 134), wherein the highlighted residue is invariant.

As used herein, each “beta strand” may be any suitable series of amino acids that include alternating hydrophobic and polar amino acid residues (in whole or in part). In some embodiments, each beta strand independently is between 8-12, 8-11, 8-10, 8-9, 9-12, 9-11, 9-10, 10-12, 10-11, 8, 9, 10, 11, or 12 amino acid residues in length when not including a functional domain, as discussed below.

As used herein, each “beta turn” may be any suitable sequence that can serve to transition between two beta strands in the polypeptide. In various embodiments, each beta turn may independently be 3-5, 4-5, 3, 4, or 5 amino acids in length when not including a functional domain, as discussed below. In other embodiments, one or more beta turn may include a proline residue.

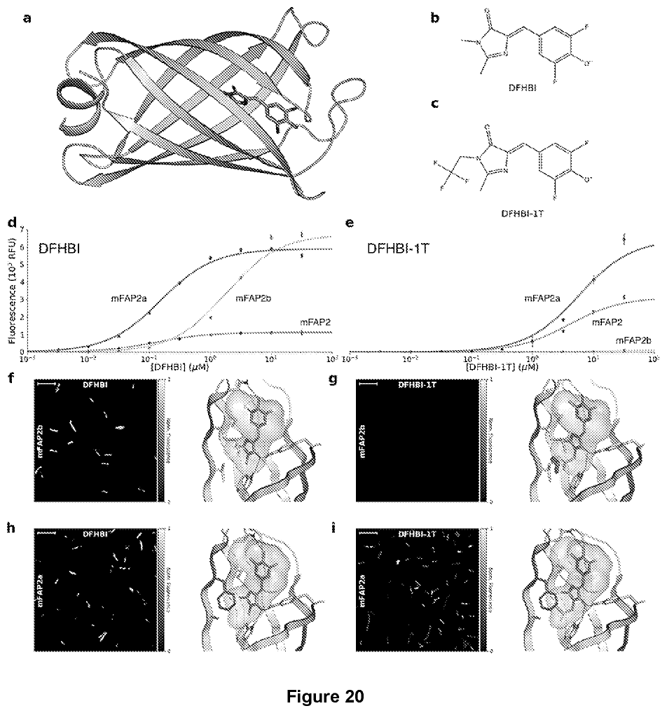

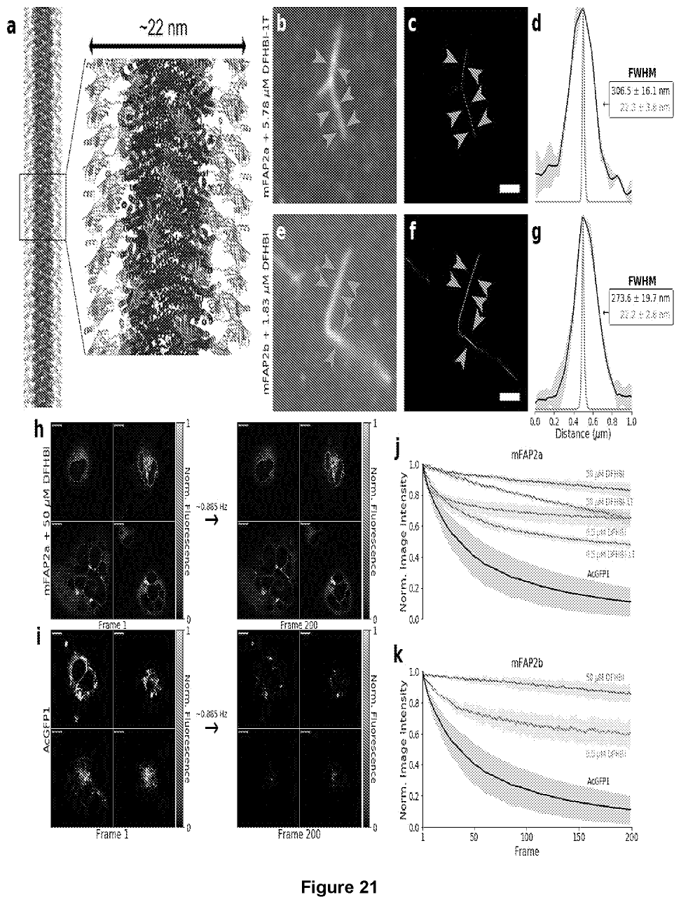

In various non-limiting embodiments, the various domains may include the following, based on the alignments shown in FIGS. 20 and 21:
X2 comprises the amino acid sequence at least 50%, 55%, 60%, 65%, 70%, 75%, 80%, 85%, 90%, 95%, or 100% identical to the amino acid sequence G (T/K/N/D) WQZT(M/F)TN (SEQ ID NO: 122) wherein Z is any amino acid, or GTWQ(V/L/A/I) T(M/F)TN (SEQ ID NO: 135), wherein the highlighted residues are invariant;
X3 comprises the amino acid sequence (E/S)DG or EDG;
X4 comprises the amino acid sequence at least 50%, 55%, 60%, 65%, 70%, 75%, 80%, 85%, 90%, 95%, or 100% identical to the amino acid sequence QTSQGQMHFQP (SEQ ID NO: 123), wherein the highlighted residues are invariant;
X5 comprises a single polar amino acid selected from the group consisting of R, T, Q, N, K, E, D, S, or wherein X5 is R;
X6 comprises the amino acid sequence (T/S)PZ3, where Z3 is polar amino acid or Tyr; or wherein X6 is SPY;
X7 the amino acid sequence at least 50%, 55%, 60%, 65%, 70%, 75%, 80%, 85%, 90%, 95%, or 100% identical to the amino acid sequence T(L/A/M)D(I/V)(K/V)(A/S) GT(I/M) (SEQ ID NO: 124) or TMDIVAQGTI (SEQ ID NO: 136), wherein the highlighted residues are invariant;
X8 comprises the amino acid sequence (S/A)DG or SDG;
X9 comprises the amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO: 125) or RPIVGYGKATV (SEQ ID NO:137), wherein the highlighted residues are invariant;
X10 is selected from the group consisting of R, T, Q, N, K, E, D, or S; or X10 is K;
X11 comprises the amino acid sequence (S/T)(P/C)(polar or Y), or wherein X 11 is TPD;
X12 comprises the amino acid sequence at least 50%, 55%, 60%, 65%, 70%, 75%, 80%, 85%, 90%, 95%, or 100% identical to the amino acid sequence T(M/L/V)(D/H/Q/N)(V/A/L/I)(D/N/H/Q)(I/L/V) T(Y/W) (SEQ ID NO: 126) or TLDIDITY (SEQ ID NO:138);
X13 comprises the amino acid sequence (S/E)DG, or wherein X13 comprises the amino acid sequence at least 60%, 80%, or 100% identical to PSLGN (SEQ ID NO: 127);
X14 the amino acid sequence at least 50%, 55%, 60%, 65%, 70%, 75%, 80%, 85%, 90%, 95%, or 100% identical to the amino acid sequence (K/M/I/L)(Q/K)(V/A/G)QGQ(V/I)T(M/L/Y) (SEQ ID NO: 128) or IKAQGQITM (SEQ ID NO: 139), wherein the highlighted residues are invariant;
X15 is selected from the group consisting of R, T, Q, N, K, E, D, or S, or wherein X15 is D;
X16 comprises the amino acid sequence (S/T)P(D/T/Y); of wherein X16 comprises the amino acid sequence SPT;
X17 comprises the amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/F/V/Y) (SEQ ID NO: 129) or QFKFDATT (SEQ ID NO: 140);
X18 comprises the amino acid sequence selected from the group consisting of (S/E/N/A/Q)DG, SDG and K(G/Q/K/T)(A/D/E/N)(G/D/N)(N/G/D/Y/S) (SEQ ID NO: 130), KG(A/D/E)(G/D/N)(N/G/D/Y) (SEQ ID NO: 131), KGENDFHG (SEQ ID NO: 141), KGADGWHG (SEQ ID NO: 142), and KGAGNFTG (SEQ ID NO: 143); and/or
X19 comprises the amino acid sequence at least 50%, 55%, 60%, 65%, 70%, 75%, 80%, 85%, 90%, 95%, or 100% identical to the amino acid sequence [(S/K/N/H)](K/R/I/N)(V/L)TGT(L/I/M)QRQE (SEQ ID NO: 132) or RLTGTLQRQE (SEQ ID NO: 144), wherein the position in brackets is optional.

As described herein, the polypeptides of the disclosure are excellent scaffolds for ligand binding. Thus, in another embodiment the polypeptides of any embodiment of the disclosure may further comprise one or more functional domains. As used herein, a “functional domain” is any polypeptide or post-translational modification that has an activity that adds functionality to the polypeptides of the disclosure. In non-limiting embodiments, such functional domains may comprise one or more polypeptide antigens, polypeptide therapeutics, ion-binding polypeptides (including but not limited to calcium-binding polypeptides), small-molecule binding polypeptides, inorganic or organic substrate-binding polypeptides, pH-sensitive polypeptides, voltage-sensitive polypeptides, mechanically-sensitive polypeptides, thermally-responsive polypeptides, nucleic acid-binding polypeptides, luminescent or fluorescent polypeptides, fluorescence quenching polypeptides, detectable markers including but not limited to covalent linking or non-covalent interaction of fluorescent molecules, luminescent or fluorescent or fluorescence quenching proteins or functional portions thereof, etc. The one or more functional domains may be fused at any appropriate regions within the polypeptides of the disclosure. In various embodiments, the one or more functional domains may be fused to one or more of the beta turn domains (i.e.: X3, X6, X8, X11, X13, X16, and/or X18). In one specific embodiment, X18 comprises a functional domain. In various other embodiments, the capping domain and/or X19 may comprise a functional domain.

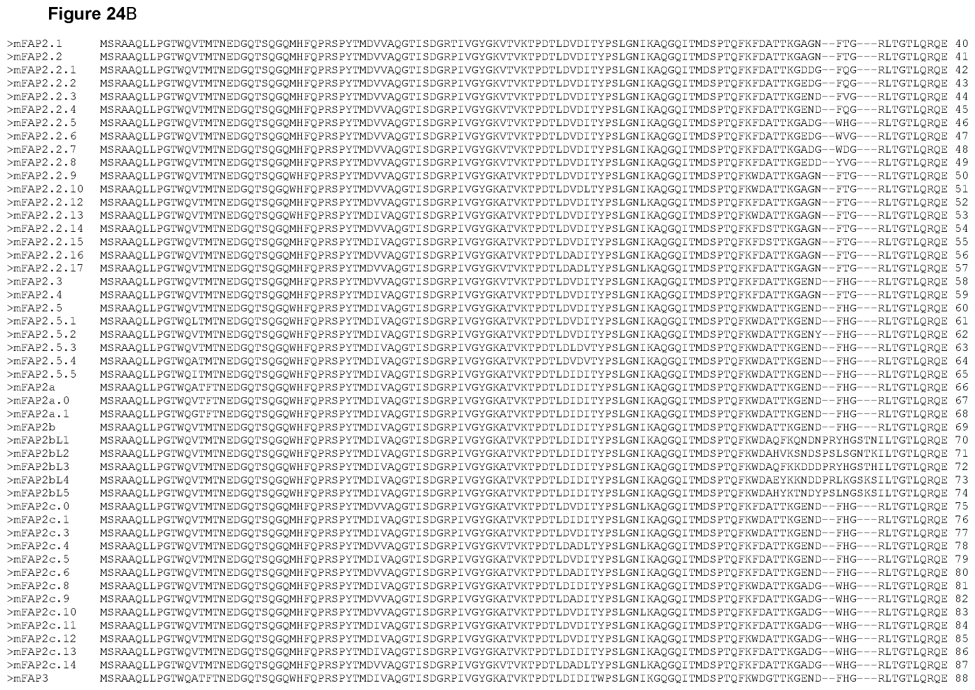

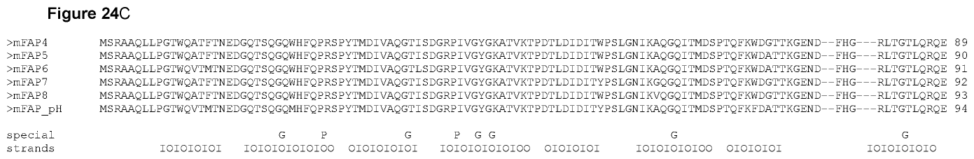

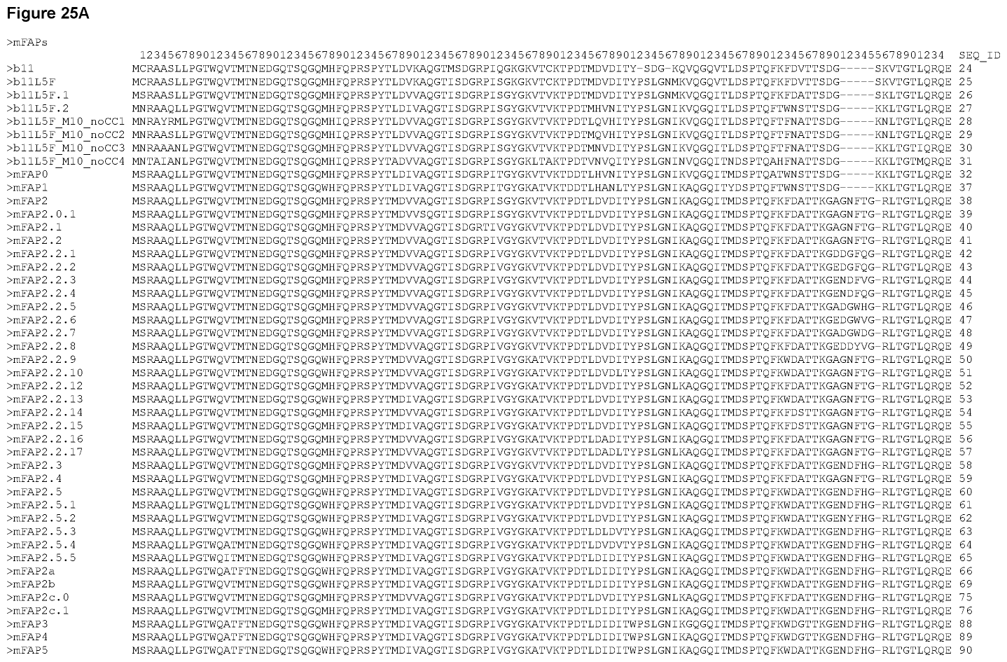

In various further embodiments, the polypeptide comprises the amino acid sequence at least 50%, 55%, 60%, 65%, 70%, 75%, 80%, 85%, 90%, 95%, or 100% identical to the amino acid sequence selected from SEQ ID NOS: 1-94, as shown in FIG. 24A-C. In all cases, the N-terminal methionine is optional.

In various further embodiments, the polypeptide comprises the amino acid sequence at least 50%, 55%, 60%, 65%, 70%, 75%, 80%, 85%, 90%, 95%, or 100% identical to the amino acid sequence selected from SEQ ID NOS:95-120, shown below. Each of these embodiments are calcium-sensing polypeptides that are based on the polypeptides in FIG. 24A-C, but have an insertion of 1 (EF1), 2 (EF2) or 4 (EF4) EF-hand motif(s) (an exemplary functional domain) in the last beta-turn (X18).

SEQ ID NO: 95
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQTKILTGTLQRQE
SEQ ID NO: 96
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQP
SEQ ID NO: 97
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQPHILTGTLQRQE
SEQ ID NO: 98
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQPRLTGTLQRQE
SEQ ID NO: 99
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQPRLTGTLQRQE
SEQ ID NO: 100
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGDGYISAAEAAAQTKILTGTLQRQE
SEQ ID NO: 101
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHYKGDKDGDGYISAAEAAAQILTGTLQRQE
SEQ ID NO: 102
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAEYKGDKDGDGYISAAEAAAQILTGTLQRQE
SEQ ID NO: 103
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAEYKGDKDGDGYISAAEAAAQILTGTLQRQE
SEQ ID NO: 104
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHYKGDKDGDGYISAAEAAAQGLTGTLQRQE
SEQ ID NO: 105
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHYKGDKDGDGYISAAEAAAQGLTGTLQRQE
SEQ ID NO: 106
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHYKGDKDGDGYISAAEAAAQILTGTLQRQE
SEQ ID NO: 107
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNYEEFVQMP
R-LTGTLQRQE
SEQ ID NO: 108
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAEEFVQMP
R-LTGTLQRQE
SEQ ID NO: 109
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAEEFVQMP
RLTGTLQRQE
SEQ ID NO: 110
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAAEAAQMT
KILTGTLQRQE
SEQ ID NO: 111
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAAEAAQMT
KILTGTLQRQE
SEQ ID NO: 112
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDATTKGDKDGNGYISAAELRHVMTNLGEKLTDEEVDEMIREADIDGDGQVNAAEAAA
MTKILTGTLQRQE
SEQ ID NO: 113
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDATTKGDKDGNGYISAAELRHVMTNLGEKLTDEEVDEMIREADIDGDGQVNAAEAAA
MTKILTGTLQRQE
SEQ ID NO: 114
SRAAQLLPGTWQVTMTNEDGQTSQGQWHFRPRSPYTMDIVAQGTISDGRPIVGYGKATVKTPDTLDIDITYPSLG
NIKAQGQITMDSPTQFKWDAHVKGDKDGNGYISAAELRHVMTNLGEKLTDEEVDEMIREADIDGDGQVNYEEFVQ
MPRLTGTLQRQE
SEQ ID NO: 115
SRAAQLLPGTWQATFTNEDGQTSQGQWHFRPRSPYTMDIVAQGTISDGRPIVGYGKATVKTPDTLDIDITYPSLG
NIKAQGQITMDSPTQFKWDAHVKGDKDGNGYISAAELRHVMTNLGEKLTDEEVDEMIREADIDGDGQVNYEEFVQ
MPRLTGTLQRQE
SEQ ID NO: 116
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAAEAAAMP
RLTGTLQRQE
SEQ ID NO: 117
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNAAEAAAMP
RLTGTLQRQE
SEQ ID NO: 118
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVDKDGNGYISAAELRHVMTNLGEKLTDEEVDEMIREADIDGDGQVNYEEFVQMP
RLTGTLQRQE
SEQ ID NO: 119
SRAAQLLPGTWQVTMTNEDGQTSQGQWHFQPRSPYTMDIVAQGTISDGRPIVGYGKATVKTPDTLDIDITYPSLG
NIKAQGQITMDSPTQFKWDAHVKGDKDGDGTITTKELGTVMRSLGQNPTEAELQDMINEVDADGDGTIDFPEFLT
MMARKMKDTDSEEEIREAFRVFDKDGNGYISAAELRHVMTNLGEKLTDEEVDEMIREADIDGDGQVNYEEFVQMP
HILTGTLQRQE
SEQ ID NO: 120
SRAAQLLPGTWQATFTNEDGQTSQGQWHFQPRSPYTMDIVAQGTISDGRPIVGYGKATVKTPDTLDIDITYPSLG
NIKAQGQITMDSPTQFKWDAHVKGDKDGDGTITTKELGTVMRSLGQNPTEAELQDMINEVDADGDGTIDFPEFLT
MMARKMKDTDSEEEIREAFRVFDKDGNGYISAAELRHVMTNLGEKLTDEEVDEMIREADIDGDGQVNYEEFVQMP
HILTGTLQRQE

In one embodiment, residues noted as “special” residues in the figures are invariant. The figure indicates residues on the interior (I) and exterior (O) of the polypeptide; residues on the exterior are readily substitutable. In other embodiments, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

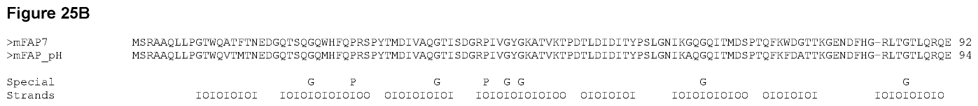

In various further embodiments, the polypeptide comprises the amino acid sequence at least 50%, 55%, 60%, 65%, 70%, 75%, 80%, 85%, 90%, 95%, or 100% identical to the amino acid sequence selected from SEQ ID NOS: SEQ ID NOS: 24-32, 37-66, 69, 75-76, 88-90, 92, and 94, as shown in FIG. 25A-B. These polypeptides have the strongest binding and/or fluorescent activities of the polypeptides disclosed herein. In all cases, the N-terminal methionine is optional.

In one embodiment, the polypeptide comprises the amino acid sequence at least 50%, 55%, 60%, 65%, 70%, 75%, 80%, 85%, 90%, 95%, or 100% identical to the amino acid sequence selected from SEQ ID NO: 38 (mFAP2).

mFAP2
(SEQ ID NO: 38)
(M)SRAAQLLPGTWQVTMTNEDGQTSQGQMHFQP
RSPYTMDVVAQGTISDGRPISGYGKVTVKTPDTL
DVDITYPSLGNIKAQGQITMDSPTQFKFDATTKG
AGNFTGRLTGTLQRQE.

In further embodiments, the polypeptide comprises residues at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or all 45 of the following positions relative to SEQ ID NO: 38 (mFAP2), with numbering starting from the first residue after the optional N-terminal methionine residue:

| Position (mFAP2 numbering, no M) | Residues |
|---|---|
| 13 | V, A, L, I |
| 15 | M, F |
| 17 | N |
| 23 | S, T |
| 27 | W, M |
| 29 | F, I |
| 37 | M, L |
| 39 | I, V |
| 41 | A |
| 45 | I, M, L |
| 49 | R |
| 50 | P, T |
| 51 | I |
| 52 | V, S, Q, T |
| 57 | A, V, L |
| 59 | V, A, C |
| 65 | L, M, V |
| 67 | I, V, A |
| 69 | I, L |
| 71 | Y, W |
| 72-76 | PSLGN (SEQ ID |
| | NO: 127) |
| 77 | I, M, L |
| 79 | A, G, V |
| 83 | I, V |
| 85 | M, L, Y, N |
| 91 | F, A |
| 93 | W, F |
| 95 | A, G, S |
| 97 | T |
| 98-100 | KG (E/A) |
| 101-103 | (N/G/D) (D/N/G) F |
| 104-106 | (H/T/Q) GR |
| 105 | F, W, Y |
| 107 | L, V |
| 111 | L, I, M |

In all of these embodiments other than SEQ ID NOS:95-120 (which include one or more functional domains), the percent identity requirement does not include any additional functional domain that may be incorporated in the polypeptide. For example, if the functional domain is incorporated in the X18 turn, then the percent identity requirement is based on the X1-17 domain, the X18 domain that does not include the functional domain, and the X19-X20 domain.

As used throughout the present application, the term “polypeptide” is used in its broadest sense to refer to a sequence of subunit D- or L-amino acids, including canonical and non-canonical amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another aspect the disclosure provides nucleic acids encoding the polypeptide of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the present disclosure provides pharmaceutical compositions, comprising one or more polypeptides, nucleic acids, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, nucleic acids, expression vectors, and/or host cells may be the sole active agent in the pharmaceutical composition, or the composition may further comprise one or more other active agents suitable for an intended use.

The polypeptides, nucleic acids, expression vectors, host cells, and pharmaceutical compositions of the disclosure may be used for any suitable purpose, as described in detail herein. In various non-limiting embodiments, the purpose may include pH sensing, ion-sensing/detection (including but not limited to Ca²⁺, La³⁺, Tb³⁺, and other ion sensing/detection/quantification), super-resolution microscopy, localization microscopy, and detection and quantification of other small-molecules, ions, inorganic or organic substrates or materials, peptides, or nucleic acids by insertion of their respective binding peptides into the loops of the polypeptides.

In another aspect the disclosure provides methods for designing beta barrel polypeptides, comprising any embodiment or combination of embodiments of polypeptide design steps disclosed herein. Detailed disclosure on such design protocols are provided in the examples that follow.

### Examples

### Background

Up-and-down beta barrels are excellent scaffolds for ligand binding as the base of the barrel can accommodate a hydrophobic core to provide overall stability, and the top of the barrel a recessed cavity for ligand binding flanked by loops which can contribute further binding affinity and selectivity. We hypothesized that the volume of the beta-barrel cavity and its 3D shape—or the shape of the cross-section perpendicular to the main axis of the barrel—can be encoded in the 2D blueprint by placing glycine kinks. The kinks would locally bend the beta-sheet into “corners” and shape an otherwise roughly circular cross-section into a polygon. Low energy amino acid sequences were obtained for backbones generated according to the above criteria using the Rosetta sequence design. Monomeric BB1 design exhibited a characteristic beta-sheet far UV circular dichroism signal, and a strong near-UV signal suggesting an organized tertiary structure. The design was stable and cooperatively folded: the circular dichroism spectrum was unchanged at 95 degrees C., and in guanidine denaturation experiments followed by both the near UV CD signal and by tryptophan fluorescence a single cooperative unfolding transition was observed at 2.5M guanidine. Having determined the rules for de novo design of beta barrels, we next sought to design functional beta barrels with cavities custom built to bind a particular ligand. We chose as a model compound 3,5-difluoro-4-hydroxybenzylidene imidazolinone (DFHBI), a close derivative of GFP chromophore that due to internal torsional flexibility, does not fluoresce upon photon excitation. Non-covalent interactions that constrain DFHBI in a planar conformation can considerably increase its fluorescence. We developed a new “Rotamer Interaction Field (RIF)” docking method that simultaneously samples over rigid body and sequence degrees of freedom using a hierarchical grid based approach. We then used a two-step Monte Carlo-based Rosetta design protocol to optimize the total complex energy. Synthetic genes encoding the 56 designs were obtained and the proteins expressed in E coli. 38 of the proteins were well expressed and soluble; sizing chromatography and far-UV circular dichroism spectroscopy showed that 22 of these were monomeric beta sheet proteins. The crystal structure of one of the non-disulfide designs (b10) was solved to 2.1 Å, and was found to have only 0.57 Å backbone RMSD from the design model. The upper barrel of the crystal structure maintains the designed pocket, and is filled with six water molecules in the absence of DFHBI. Thus, the design principles are sufficiently robust to allow the accurate design of potential binding pockets. Three of the 22 monomeric designs were found to activate DFHBI fluorescence. With DFHBI bound, b11 and b32 have the characteristic emission spectra of eGFP with an absorption peak at 450 nm and an emission peak at 510 nm. Knockout of the designed interacting residues in the binding pocket eliminates the 510 nm fluorescence. We sought to improve interactions with the ligand by redesigning the top beta turns around the ligand binding site, and introducing turn substitutions to make additional ligand contacts. One such variant b11_L5F with its fifth turn changed to a five-residue turn increased the fluorescence intensity by fourfold.

To obtain a comprehensive view of the sequence determinants of both the beta barrel scaffold and the conformation-specific DFHBI binding activity, we assayed every possible point mutant of b11_L5F for protein stability and fluorescence activation. A library containing all the point mutants of b11_L5F was displayed on the yeast cell surface. To further improve the fluorescence activation, we constructed a site-directed mutagenesis library using doped DNA oligos that incorporated dual or single beneficial mutations at 20 positions.

A) De Novo Design of Fluorescence-Activating Beta Barrels

Protein structures rarely have internal symmetry, and hence the symmetric twisting of alpha helices around a central axis in coiled coils and of beta strands in beta barrels has fascinated scientists since they were first discovered. Here we first show that accurate de novo design of beta barrels requires considerable symmetry breaking to achieve continuous hydrogen bond connectivity and eliminate backbone strain. We then build ensembles of beta barrel backbone structures with cavity shapes matched to the fluorogenic compound DFHBI, and use a hierarchical grid-based search method to simultaneously optimize the rigid body placement of DFHBI in these cavities and the identities of the surrounding amino acids for high shape and chemically complementary binding. The designs have high structural accuracy and bind and fluorescently activate DFHBI in vitro and in E. coli, yeast and mammalian cells. This de novo design of small molecule binding activity, using backbones custom built to bind the ligand, permits design of increasingly sophisticated ligand binding proteins, sensors, and catalysts that are not limited by the backbone geometries available in known protein structures.

Two outstanding unsolved challenges remain in designing protein folds from scratch. First, the de novo design of all-β proteins, which is complicated by the tendency of β-strands and sheets to associate intermolecularly to form amyloid like structures if their register is not perfectly controlled. Second, the design of protein backbones customized to bind small molecules of interest, which requires precise control over both backbone and sidechain geometry, as well as balancing the sometimes opposing requirements of protein folding and function. Success in developing such methods would reduce the longstanding dependency on natural proteins, enable protein engineers to craft new proteins optimized to bind chosen small-molecule targets, and lay a foundation for de novo design of proteins customized to catalyze specific chemical reactions.

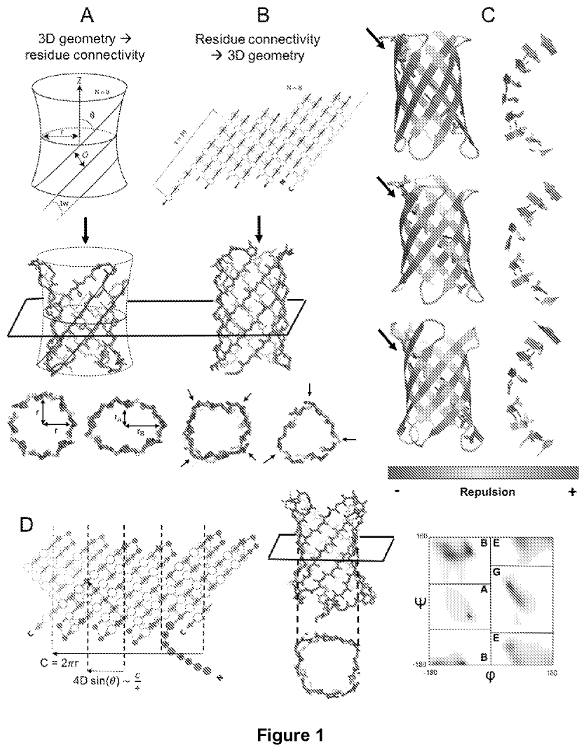

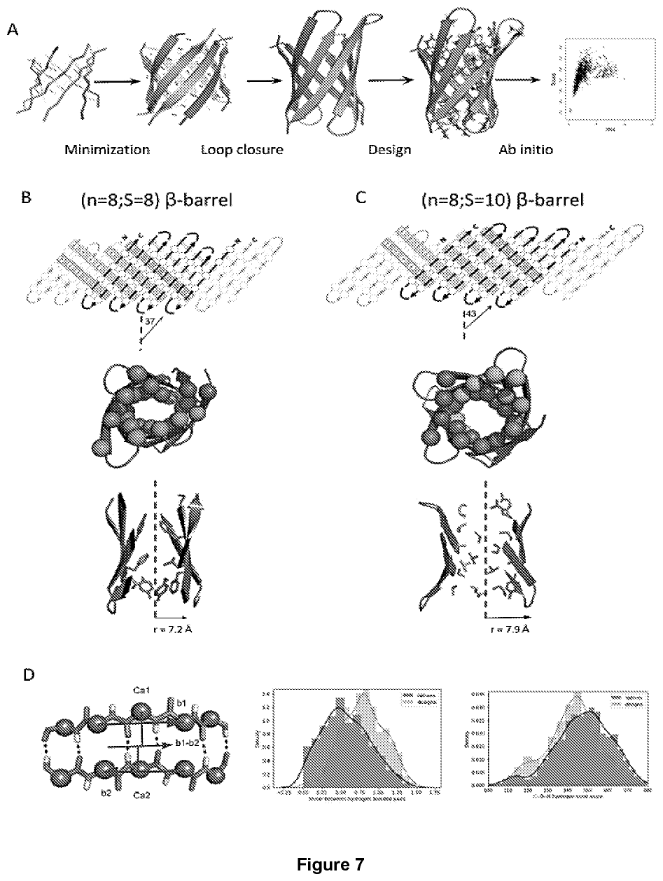

Anti-parallel β-barrels are excellent scaffolds for ligand binding, as the base of the barrel can accommodate a hydrophobic core to provide overall stability, and the top of the barrel provides a recessed cavity for ligand binding (often flanked by loops which can contribute further binding affinity and selectivity). We first set out to address this problem by parametrically generating regular arrangements of 8 anti-parallel β-strands using the equations for an elliptic hyperboloid of revolution. We generated ensembles of backbones by sampling the elliptical parameters and the tilt of the strands with respect to the barrel axis (FIG. 1A, top) arranging the Cαs along equidistant straight lines on the hyperboloid surface (FIG. 1A, center). Backbones generated with such constant coil angles between strands could not achieve perfectly regular hydrogen bonding. To resolve this problem, we introduced force-field guided variation in local twist by gradient based minimization (see Methods). We selected the backbones with the most extensive inter-strand hydrogen bonding, connected the strands with short loops and carried out combinatorial sequence optimization to obtain low energy sequences (FIG. 7A). Synthetic genes encoding 41 such designs were produced and the proteins expressed in E. coli. Almost all were found to be insoluble or oligomeric; none of this first set of 41 designs were monomeric with an all-β circular dichroism spectrum.

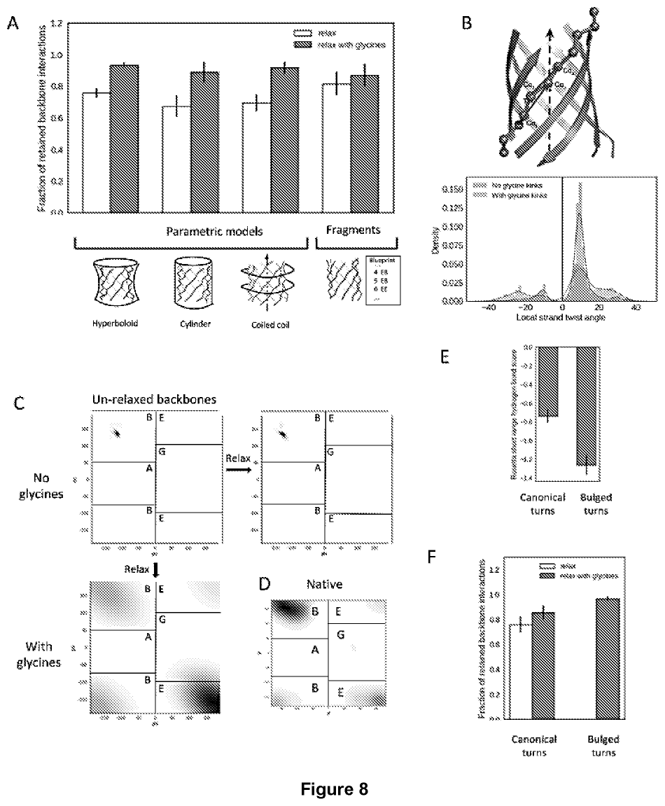

In considering the reasons for the failure of the initial designs, we noted that many of the backbone hydrogen bond interactions on the top and bottom of the barrels were distorted or broken (FIG. 7D). To investigate the origins of this distortion, we experimented with three alternative approaches to generating symmetric beta barrel backbones (without loops, see Methods) and observed strand splitting in all cases following iterative minimization and sidechain repacking with Rosetta (FIG. 8A), suggesting there is strain inherent to the closing of the curved β-sheet on itself. To identify the origin of this strain, we repeated the relaxation after imposing strong constraints on the hydrogen bond interactions to prevent them from breaking. As illustrated in FIG. 1C, the strain manifested in two places. First, steric clashes build up along strips of side-chains in the directions of the hydrogen bonds, perpendicularly to the direction of the β-strands (“Cβ-strips”) (FIG. 1C, top right). Second, unfavorable left handed twist appears in β-strand residues (FIG. 8B; the chirality of the peptide backbone favors right handed twist).

These results indicate that large local deviations in ideal β-strand twist are necessary to maintain continuous hydrogen bond interactions between strands in a closed β-barrel, and hence that a parametric approach assuming uniform geometry may not be optimal. Instead, we chose to build β-barrel backbones starting from a 2D map specifying the peptide bonds, the backbone torsion angle bins, and the backbone hydrogen bonds (FIG. 1B). We then used this map to drive the assembly with Rosetta™ of a 3D model from an extended peptide chain. In contrast to parametric backbone design, which may be viewed as “3D to 2D” approach as a 3D surface is generated and then populated with residues (FIG. 1A), this alternative strategy proceeds from 2D to 3D and can readily incorporate local torsional deviation. The definition of hydrogen bond interactions in the 2D map also enables direct control over the β-barrel sheer number (S)—the total shift in strand registry between the first and last strand—which defines the hydrophobic packing arrangement and the diameter of the barrel (FIG. 7B).

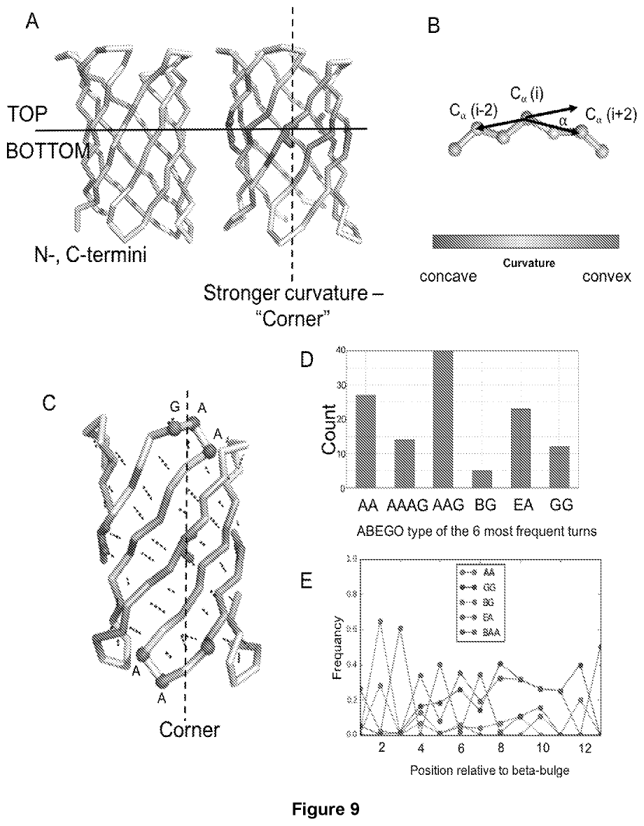

We found that both the steric and left-handed twist related issues could be solved by strategic placement of glycine residues (which are normally disfavored in beta sheets^{20,21}) and β-bulges in the 2D map. The achiral glycine residues can have a left-hand twist without disrupting the β-sheet hydrogen bond pattern and reduce the steric clashes within Cβ-strips (FIG. 1C, middle). We also found that β-bulges associated with β-turns reduce steric strain at the extremities of Cβ-strips (FIG. 1C, bottom) and stabilize the hydrogen bonds between the β-strand residues flanking the turns (FIG. 8E). The breaking of backbone hydrogen bonds observed during relaxation of the uniform poly-valine models was eliminated by incorporation of one glycine with defined positive Φ torsion bin (“glycine kinks”, region E in FIG. 8D) in each of the 5 Cβ-strips of an 8 stranded (n=8) S=10 β-barrel, and β-bulges near the β-turns (FIG. 8A, F, FIG. 9).

We were able to control the volume and the shape of the β-barrel cavity by altering the placement of glycine kinks in the 2D map. Such kinks dramatically increase local β-sheet curvature, forming corners in an otherwise roughly circular cross-section (FIG. 1B, bottom). We chose to design a square barrel shape and placed 5 “strain-relieving” and 1 “shaping” glycine based on the geometric parameters of (n=8; S=10) β-barrels (FIG. 1A). The resulting 3D backbones feature a large and regular ligand-binding cavity volume (FIG. 1D). To tie together the bottom of the barrel, we introduced a “tryptophan corner” by placing a short 3-10 helix followed by a glycine kink and a Trp at the beginning of the barrel, and an interacting Arg at the C-terminus.

Low energy amino acid sequences were designed for these backbones using Rosetta™ flexible backbone combinatorial design. Four designs with low energy and backbone hydrogen bonding and local geometry matching the 2D map were selected for experimental characterization (Table 1).

TABLE 1
Experimental characterization of nonfunctional
β-barrel designs based on the 2D map.

| Design | | E. coli | | | β CD |
|---|---|---|---|---|---|
| ID* | E-value** | Expression | Solubility | SEC | spectrum |
| BB1 | 0.1100 | yes | yes | monomer | yes |
| G9A | | yes | yes | oligomers | |
| W11A | | yes | yes | oligomers | |
| R107A | | yes | yes | oligomers | |
| BB2 | 1.1000 | yes | yes | tetramer | yes |
| BB3 | 1.9000 | yes | no | | |
| BB4 | 0.5600 | yes | yes | | no |

*G9A, W11A and R107A are the knockout mutants of the tryptophan corner in BB1.
**E-value is calculated by BLAST the nonredundent protein database.

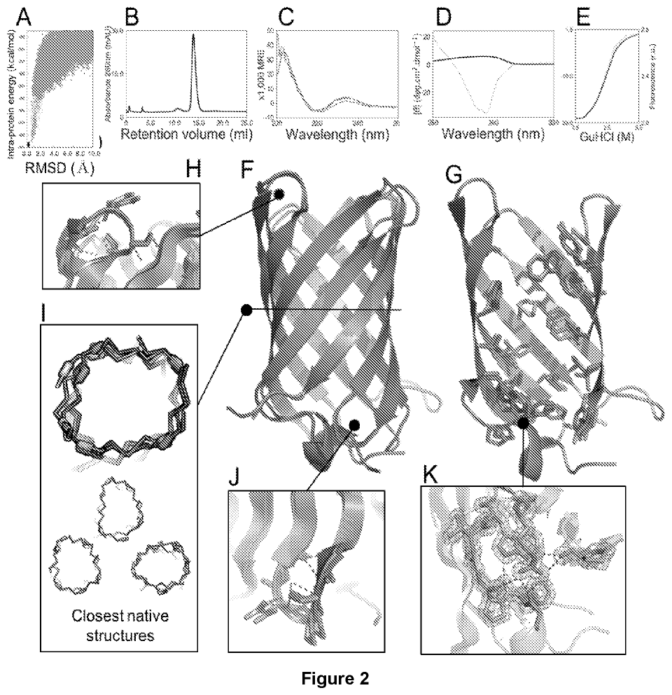

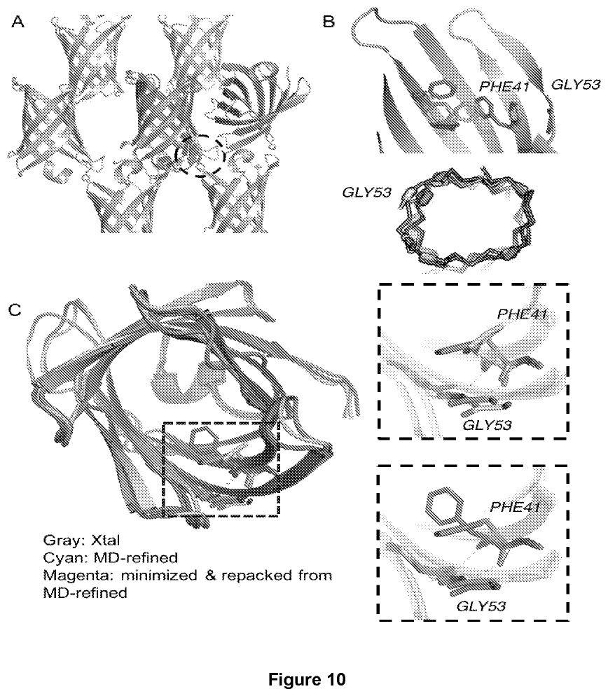

The sequences of these designs are not related to those of proteins with known structure (BLAST E-values ranged from 0.11 to 1.9 against the non-redundant protein database) and fold into the designed structure in silico (FIG. 2A). Synthetic genes encoding the designs were expressed in E. coli. Two of the designs were expressed soluble and purified; size-exclusion chromatography (SEC) coupled with multi-angle light scattering (MALS) showed that one was a stable monomer (BB1, SEQ ID NO 1) (FIG. 2B) and the other a soluble tetramer. BB1 exhibited a characteristic β-sheet far UV circular dichroism (CD) signal, and a strong near-UV signature suggesting an organized tertiary structure (FIG. 2C,D). The design was stable at 95 degree Celsius, and showed cooperative unfolding in a guanidine titration experiment (FIG. 2E). The crystal structure of BB1 solved at 1.6 Å resolution was very close to the design model (within 1.4 Å RMSD over 99 residues of the 109 residues, FIG. 2F-G, deviations from the design are shown in FIG. 10) and had no close structural homolog in the PDB (highest TM-score <0.8). Essentially all of the key features of the design model are found in the crystal structure. The cross-section of the structure resembles the square cavity of the design model, which is not observed in any existing natural β-barrel crystal structures (FIG. 21), and is shaped by the designed glycine kinks. All 7 designed turns and β-bulges are correctly recapitulated in the crystal structure (FIG. 2H,J), along with the 3-10 helix and tryptophan corner (FIG. 2K).

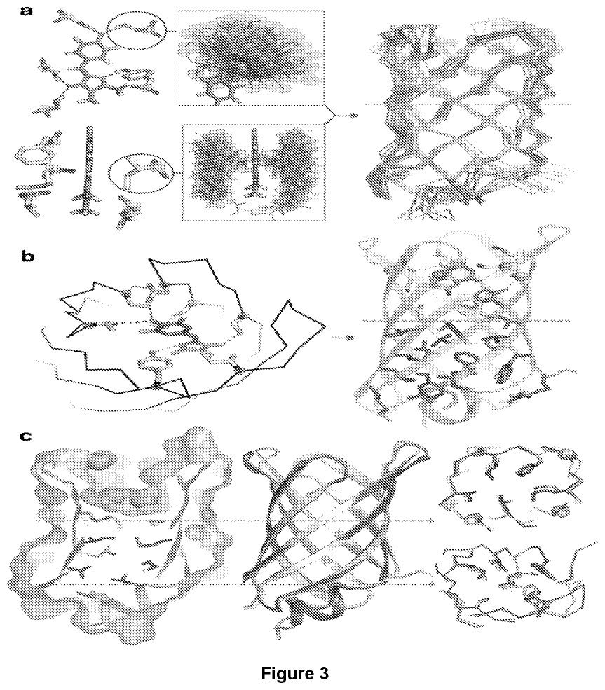

Having determined principles for de novo design of β-barrels, we next sought to design functional β-barrels with binding sites tailored for a small molecule of interest. We chose DFHBI ((Z)-4-(3,5-difluoro-4-hydroxybenzylidene)-1,2-dimethyl-1H-imidazol-5(4H)-one) (FIG. 3a, left, green), a derivative of the intrinsic chromophore of GFP, as a model compound to test the computational design methods. Due to its internal torsional flexibility, DFHBI does not fluoresce unless it is constrained in the cis-planar conformation^{25,26}. We sought to design protein sequences that fold into a stable β-barrel structure with a recessed cavity complementary in shape to DFHBI and lined with side-chains to bind and constrain DFHBI selectively in its cis-planar conformation. We chose to take a three step approach: (1) de novo construction of β-barrel backbones with suitably shaped cavities, (2), placement of DFHBI in the pocket, and (3) energy-based sequence design. For the first step, we used the 2D map developed above to construct an ensemble of 200 β-barrel backbones with cavities suitable for DFHBI binding in the upper half of the barrel (FIG. 3a, right).

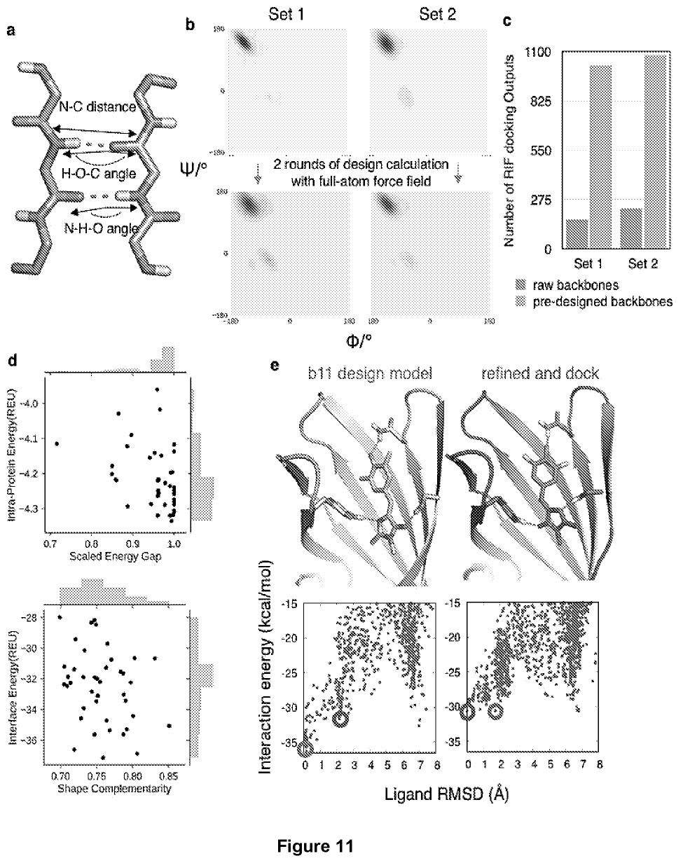

The placement of ligand in the binding pocket requires sampling of both the rigid body movement of the ligand, and the sequence identities of the surrounding amino acids that form the binding site. Because of the dual challenges associated with optimization of structure and sequence simultaneously, most approaches to designing ligand-binding site to date have separated sampling into two steps: rigid body placement of the target ligand in the protein binding pocket followed by design of the surrounding sequence. This two-step approach has the limitation that optimal rigid body placement cannot be determined independently of knowledge of the possible interactions with the surrounding amino acids. We addressed these challenges with a new “Rotamer Interaction Field (RIF)” docking method that simultaneously samples over rigid body and sequence degrees of freedom (see Methods). RIF docking first generates an ensemble of billions of discrete amino acid side chains that make hydrogen-bonding and non-polar hydrophobic interactions with the target ligand. Then, a hierarchical grid based search algorithm is used to place this pre-generated interacting ensemble in the scaffold (FIG. 3a & b). We used RIF docking to place DFHBI into the upper portion of the β-barrel for each of the 200 scaffolds in the ensemble (FIG. 3a and FIG. 11a-c).

To identify protein sequences that can not only buttress the ligand-coordinating residues from the RIF docking but also have low intra-monomer energies to drive protein folding, we developed a Monte Carlo-based sequence design protocol that iterates between 1) fixed-backbone design around the ligand-binding site to optimize ligand interacting energy and 2) flexible-backbone design for the rest of protein optimizing the total complex energy (FIG. 3b) (see Methods). This protocol was applied to 2,102 DFHBI-backbone pairs from RIF docking, and 42 designs with large computed monomer folding energy gaps and low energy protein-ligand interactions and intra-protein interactions were selected for experimental characterization, plus an additional 14 variants with a single disulfide bond added to increase stability (FIG. 11d & Table 2). Ligand docking simulations following extensive structure refinement (see Methods) revealed that due to the approximate symmetry of the hydrogen bonding pattern of DFHBI, many of the designed binding pockets could accommodate the ligand in two equally-favorable orientations (FIG. 11e).

TABLE 2
Photophysical characterization of mini- fluorescence-activating proteins (mFAPs).

| | | | | Extinction | Absolute | Relative | |
|---|---|---|---|---|---|---|---|
| | λ_{abs}(nm)* | λₑₓ(nm)* | λₑₘ(nm)* | Coefficient (M⁻¹cm⁻¹) | Quantum Yield** | Quantum Yield** | K_{d}(μM) |
| mFAP1-DFHBI | 485 | 487 | 506 | 49,300 | 0.020 | 0.022 | 0.56 ± 0.07 |
| mFAP2-DFHBI | 476 | 480 | 500 | 43,200 | 0.021 | 0.021 | 0.18 ± 0.05 |
| DFHBI | 418 | — | — | 31,935 | 0.001 | 0.0007*** | |

*λ_{abs} is peak absorbance wavelength, λₑₓ is peak excitation wavelength and λₑₘ is peak emission
**Absolute quantum yield is measured with an integrating sphere; Relative quantum yield is measured using acridine yellow and fluorescein as the standards (see Methods).
***reported value²⁵.

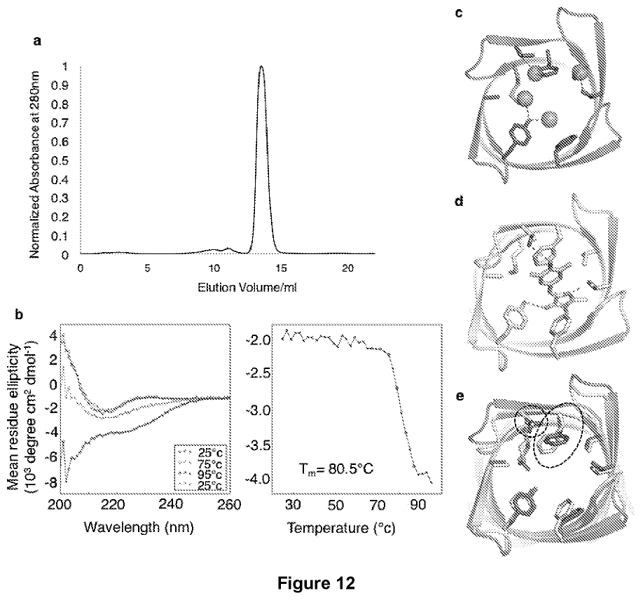

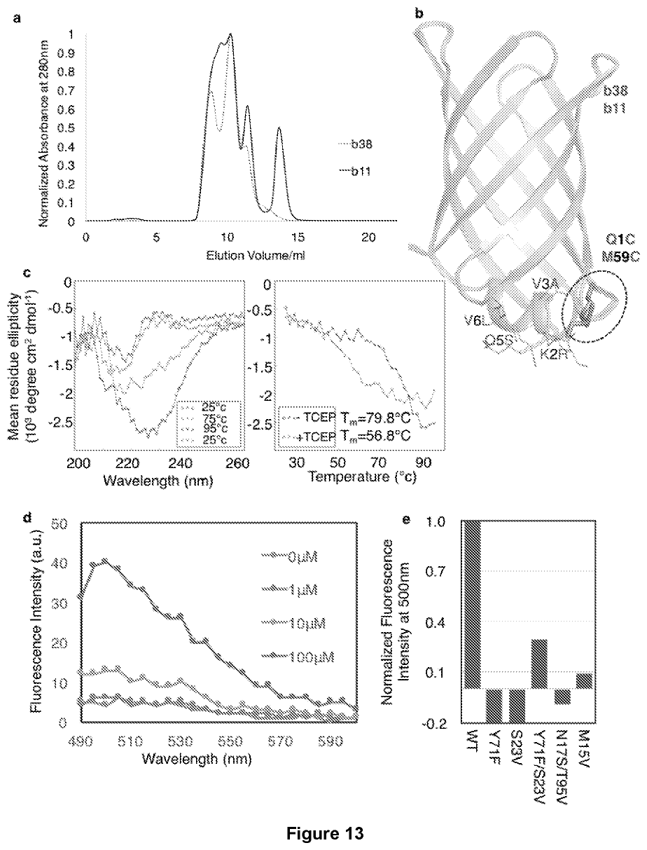

Synthetic genes encoding the 56 designs were obtained and the proteins expressed in E. coli. 38 of the proteins were well expressed and soluble; SEC and far-UV CD spectroscopy showed that 20 of these were monomeric β-sheet proteins (Table 2). Four of the oligomer-forming designs became monomeric upon incorporation of a disulfide bond between the N-terminal 3-10 helix and the barrel β-strands (FIG. 13d). The crystal structure of one of the monomeric designs (b10 SEQ ID NO:3) was solved to 2.1 Å, and was found to be very close to the design model (0.57 Å backbone RMSD) (FIG. 3c, & FIG. 12c-e). The upper barrel of the crystal structure maintains the designed pocket, which is filled with multiple water molecules (FIG. 3c, left, & FIG. 12c). Thus, the design principles described above are sufficiently robust to allow the accurate design of potential small molecule binding pockets.

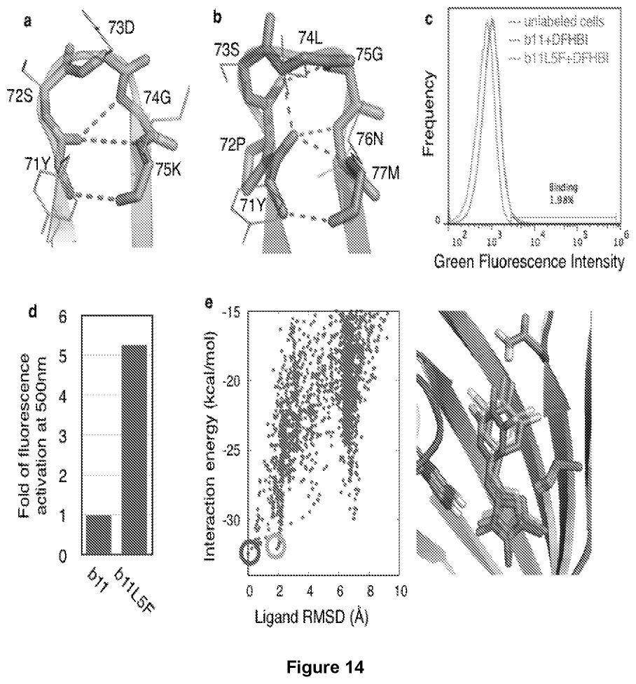

Monomeric designs b11 (SEQ ID NO:4) and b32 (SEQ ID NO:12) were found to activate DFHBI fluorescence (FIG. 13d). Knockout of interacting residues in the designed binding pocket eliminated fluorescence (FIG. 13e). The ligand-binding activity comes at a substantial stability cost as almost half of the barrel is carved out to form the binding site: while the original BB1 (designed for stability alone) does not temperature denature, both b11 and b32 undergo reversible thermal melting transitions (FIG. 13b). b11 contains a disulfide bond; the parent design lacking the disulfide (b38) is not a monomer (FIG. 13a-c, & Table 2). We sought to improve the binding interactions and fluorescence intensity by redesigning β-turns around the ligand binding site (see Methods). b11L5F (SEQ ID NO:25) with a 5-residue fifth turn showed increased fluorescence intensity (FIG. 14a-d).

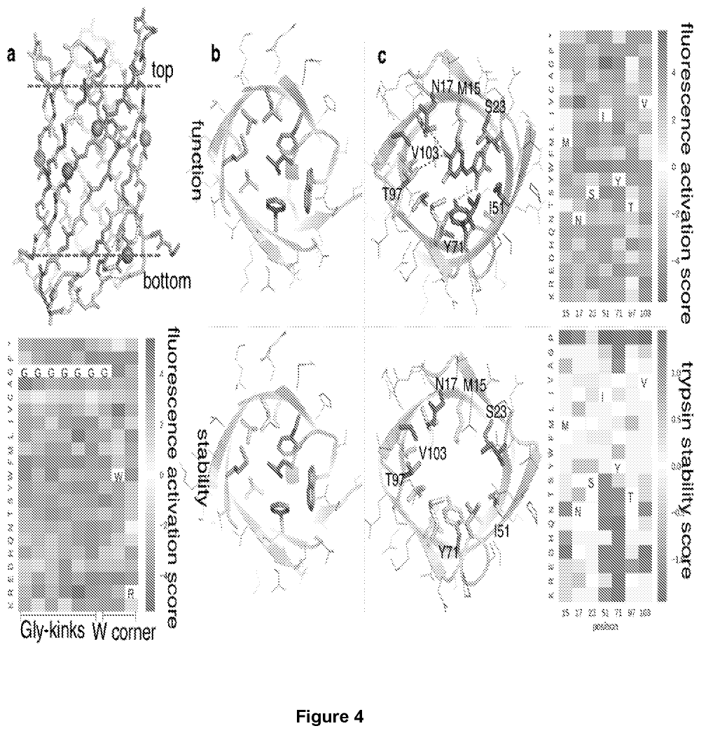

To obtain a comprehensive view of the sequence determinants of the conformation-specific DFHBI binding activity of b11L5F, we assayed the effect of each single amino acid substitution (19*110=2,090 in total) on both protein stability²⁹ and DFHBI activation on the yeast cell surface³⁰. The function (fluorescence activation) and stability (proteolysis resistance) landscapes have similar overall features consistent with the design model, with residues buried in the designed β-barrel geometry much more conserved than surface exposed residues (FIG. 4a). The function landscape suggests the geometry of the designed cavity helps activate DFHBI fluorescence: the key sequence features that specify the geometry of the cavity—the glycine kinks and the tryptophan corn-r—are conserved (FIG. 4a-b). Of the six coordinating residues from RIF docking, only a single substitution (V103L) increased fluorescence (FIG. 4c, upper right). Whereas the structure and function landscapes were very similar at the bottom of the barrel (FIG. 4b), there was a striking trade-off between stability and function at the top of the barrel around the designed binding site (FIG. 4c, left): many substitutions that stabilize the protein drastically reduce fluorescence activation (FIG. 4c, left). This bottom/top contrast indicates that success in de novo design of fold and function requires a substantial portion of the protein (in our case, the bottom of the barrel) to provide the driving force for folding as the functional site will likely be destabilizing.

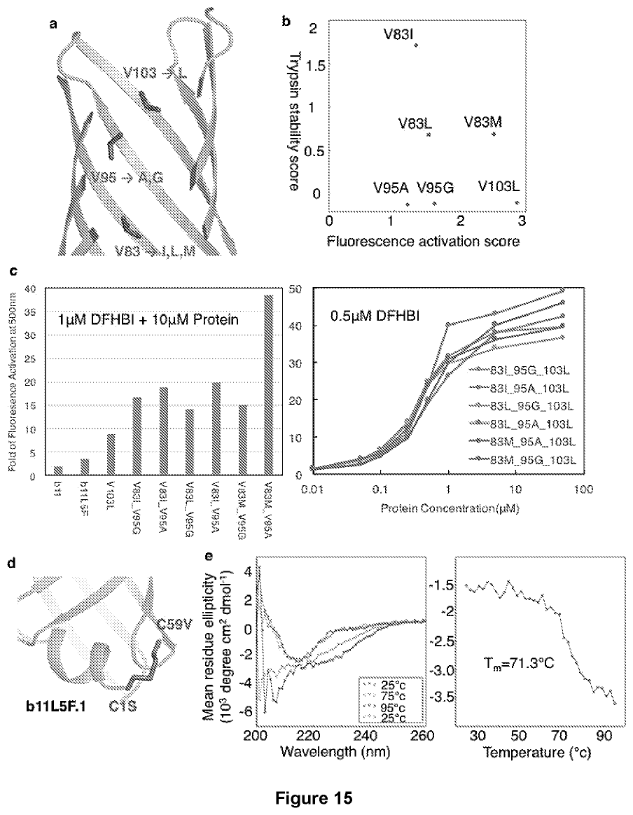

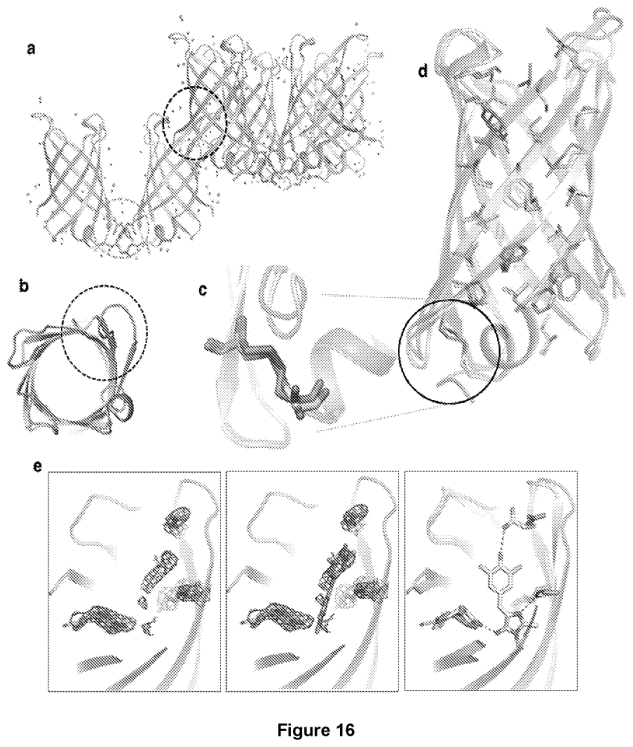

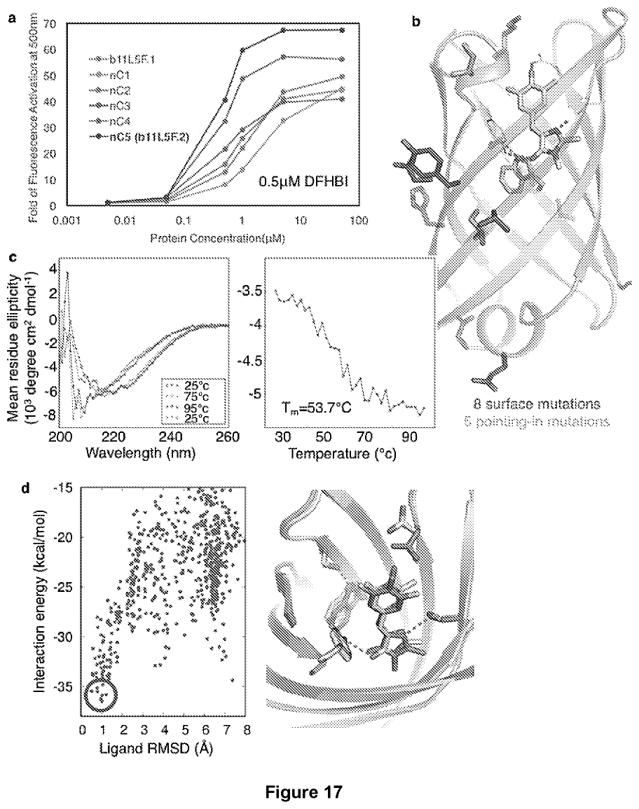

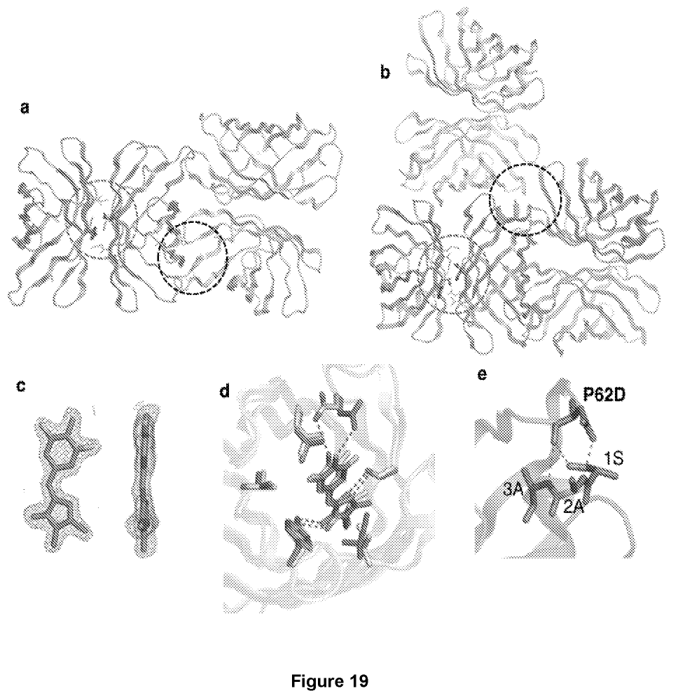

Guided by the comprehensive protein stability and fluorescence activation maps, we combined substitutions at three positions that improved function without compromising stability (V103L, V95AG and V83ILM), and obtained variants with tenfold higher DFHBI fluorescence that form stable monomers without a disulfide bond (b11L5F.1; FIG. 15a-e, SEQ ID NO:26). The crystal structure of one of these variants (b11L5F_LGL) was solved to 2.2 Å and was very close to the design model (FIG. 16a-c). The majority of the buried side chains adopt the designed rotameric conformation, however, the electron density around the DFHBI could not be resolved, consistent with the multiple binding modes suggested by the docking calculations FIGS. 16e, 11e & 14e). A second round of computational design calculations was carried out to favor a specific binding mode by optimizing the interactions with the lowest energy docked conformation, and to optimize hydrophobic packing interactions in the bottom of the barrel now freed from the disulfide bond (see Methods and FIGS. 11e (right) & 15d). Five designs predicted by ligand docking calculations to have a single ligand binding conformation were experimentally tested and three showed increased fluorescence activity, the best of which increased the fluorescence by approximately 1.4-fold (b11L5F.2 (SEQ ID NO:27); FIG. 17 a-d). Screening of two combinatorial libraries (based on b11L5F.1 and b11L5F.2) incorporating additional substitutions identified in the stability and function maps (FIG. 15) yielded variants with 1.5-to-2 fold increased fluorescence intensity and improved protein stability (FIG. 19a-c; activation of fluorescence of 0.5 μM DFHBI by approximately 100-fold). We refer to these mini-fluorescence-activating proteins as mFAPs in the remainder of the text; mFAP0 and mFAP1 are variants of b11L5F.2, and mFAP2 of b11L5F.1.

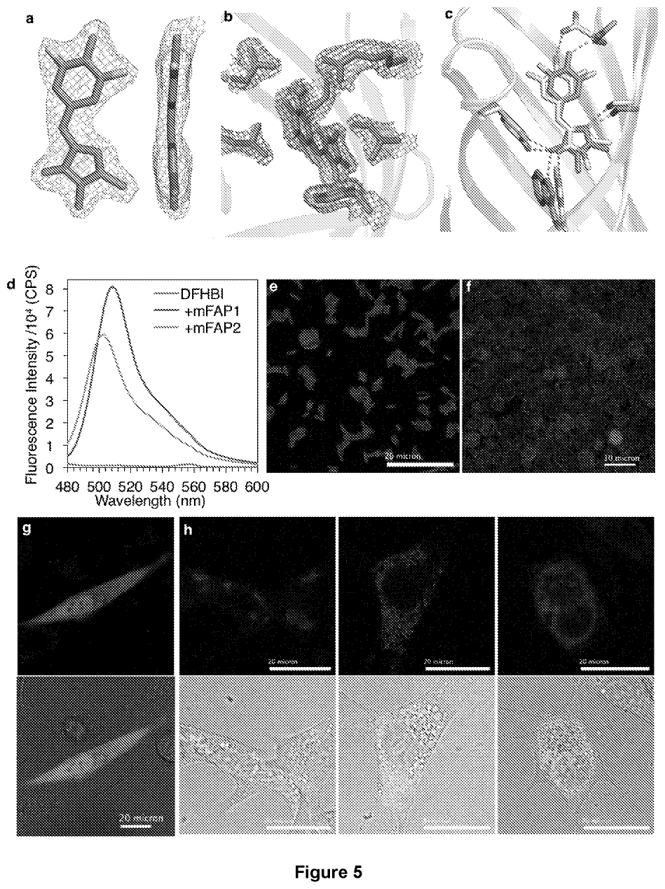

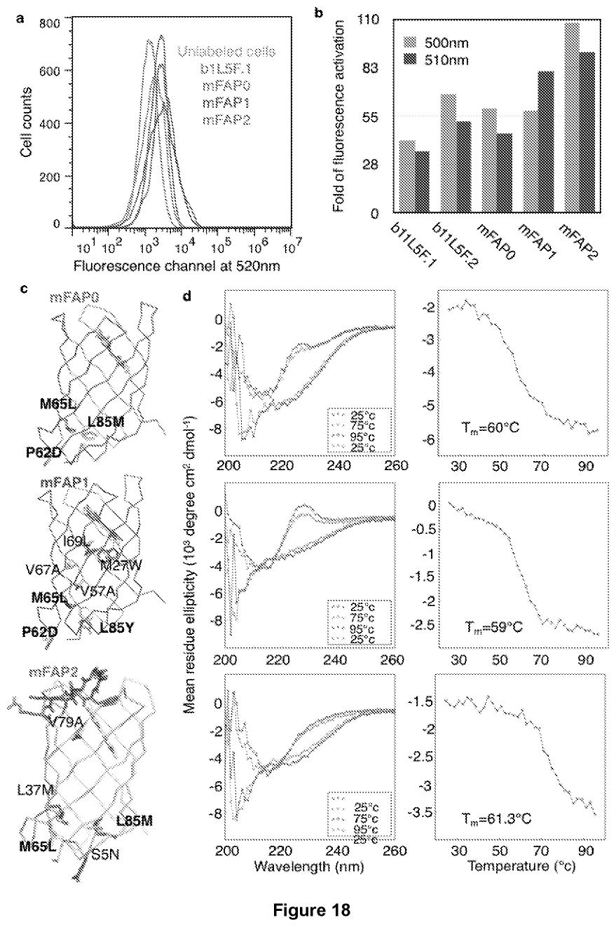

The 1.8 Å and 2.3 Å crystal structures of mFAP0 (SEQ ID NO:32) and mFAP1 (SEQ ID NO:33) in complex with DFHBI were virtually identical to the design models with an overall backbone RMSD of 0.91 Å and 0.64 Å, respectively (FIG. 5a-c & FIG. 19a-d). DFHBI is in the cis-planar conformation with unambiguous electron density in both structures (FIG. 5a-b & FIG. 19c). In addition to three designed hydrogen bonds, a water molecule was found to interact with the solvent exposed phenol group in DFHBI (FIG. 5b). The DFHBI binding modes in the crystal structures are nearly identical to the lowest-energy docked conformations used in the second round design calculations, with all-atom RMSD of 0.12 Å and 0.35 Å respectively (FIG. 5c and FIG. 19d). Three mutations shared by mFAP0 and mFAP1 in the bottom barrel (P62D, M65L and L86MorY) likely stabilize the protein by helical capping and subtle hydrophobic rearrangements (FIG. 19e). The M27W mutation in mFAP1 introduced an additional hydrogen bond to DFHBI that likely leads to a 5 nm red-shift in its fluorescence spectra (FIG. 5d). mFAP2 (SEQ ID NO:38) based on b11L5F.1 has a 6-residue insertion in the seventh β-turn (FIG. 18c-d, bottom row) that was predicted to form multiple intra-loop hydrogen bonds.

To determine whether the designed DFHBI-binding fluorescence-activating proteins function in living cells, we imaged mFAP1- and mFAP2-DFHBI complexes in E. coli, yeast, and mammalian cells by confocal microscopy. Both mFAP1 and mFAP2 showed in vivo fluorescence activation upon adding 20 μM DFHBI (FIG. 5e-h) activation was observed in less than 5 minutes following DFHBI addition). Cytosolic expression of mFAP2 in E. coli and mammalian cells showed clear fluorescence throughout the cells (FIG. 5e & g). Yeast cells displaying mFAP2 on their surfaces resulted in fluorescence in a thin region outside of the plasma membrane (FIG. 5f). Fusion of the mFAPs to mitochondrial and ER localized proteins resulted in fluorescence tightly localized to these organelles (FIG. 5h). The quantum yield of mFAP1 and mFAP2 in complex with DFHBI is 2.0% and 2.1% respectively (Table 2).

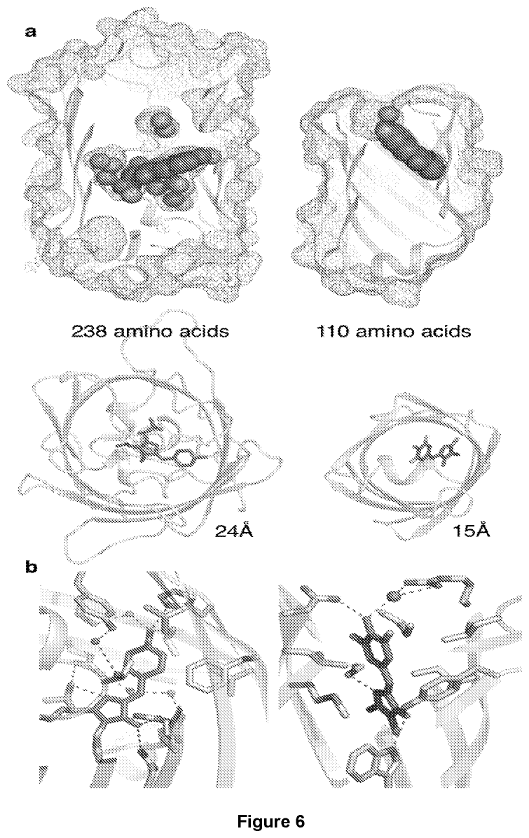

It is instructive to compare the structures of our designed fluorescence-activating proteins with those of natural fluorescent proteins (FIG. 6). Both are β-barrels, and have similar chromophores, but our designs have less than half the residues and narrower and shorter barrels connected with short β-turns (mFAP1 has 109 residues and fluorescent proteins in the GFP family are 226-263 residues) (FIG. 6a). In both cases, specific protein-chromophore interactions prevent chromophore intramolecular motions from dissipating the absorbed photons, but the hydrogen bonding and hydrophobic packing around DFHBI is different from GFP and is tailored to the smaller and simpler β-barrel (FIG. 6b). The precise structural control by computational design, together with the greater exposure of the chromophore, may prove useful for fluorescence-based imaging and sensing applications.

The comparison in FIG. 6 highlights the two primary advances: the first successful de novo design of a β-barrel, and the first de novo design of the fold and function of a small molecule binding protein. The first advance required the elucidation of general principles for designing β-barrels, notably the requirement for symmetry breaking to reduce steric strain and enable hydrogen bonding throughout the barrel structure. These principles, identified by pure geometric considerations coupled with simulations following failure of the initial parametric design approach, are borne out by both the crystal structures, which show that the designed structures are almost perfectly specified by the designed sequences, and the sequence fitness landscapes, which show that the key sequence features of the design are essential to structure and function (FIG. 4a). The second advance goes considerably beyond the design of ligand binding proteins and catalysts to date, which has relied on repurposing naturally occurring scaffolds. The three step approach taken here—first, identifying the basic principles required for specifying a general fold class, second, using these principles to generate a family of backbones with pocket geometries matched to the ligand or substrate of interest, and third, designing shape and chemically complementarity in the binding pockets by simultaneous optimization of the sequence of the binding pocket and the rigid body orientation of the small molecule using the new RIF docking approach—provides a general solution to the de novo design of binding pocket problem. The generative approach allows the exploration of an effectively unlimited set of backbone structures with shapes customized to the ligand or substrate of interest, and equally importantly, provides a critical test of our understanding of the determinants of folding and binding that goes well beyond descriptive analysis of existing protein structures.
1. Huang, P.-S., Boyken, S. E. & Baker, D. The coming of age of de novo protein design. Nature 537, 320-327 (2016).
2. Marcos, E. et al. Principles for designing proteins with cavities formed by curved β sheets. Science 355, 201-206 (2017).
3. Bick, M. J. et al. Computational design of environmental sensors for the potent opioid fentanyl. Elife 6, (2017).
4. Tinberg, C. E. et al. Computational design of ligand-binding proteins with high affinity and selectivity. Nature 501, 212-216 (2013).
5. Dou, J. et al. Sampling and energy evaluation challenges in ligand binding protein design. Protein Sci. (2017). doi:10.1002/pro.3317
6. Liu, C. et al. Out-of-register β-sheets suggest a pathway to toxic amyloid aggregates. Proc. Natl. Acad. Sci. U.S.A. 109, 20913-20918 (2012).
7. Polizzi, N. F. et al. De novo design of a hyperstable non-natural protein-ligand complex with sub-A accuracy. Nat. Chem. (2017). doi:10.1038/nchem.2846
8. De Simone, G., Ascenzi, P. & Polticelli, F. Nitrobindin: An Ubiquitous Family of All β-Barrel Heme-proteins. IUBMB Life 68, 423-428 (2016).
9. Richter, A., Eggenstein, E. & Skerra, A. Anticalins: exploiting a non-Ig scaffold with hypervariable loops for the engineering of binding proteins. FEBS Lett. 588, 213-218 (2014).
10. Toda, M., Zhang, F. & Athukorallage, B. Elastic Surface Model For Beta-Barrels: Geometric, Computational, And Statistical Analysis. Proteins (2017). doi:10.1002/prot.25400
11. Novotný, J., Bruccoleri, R. E. & Newell, J. Twisted hyperboloid (Strophoid) as a model of beta-barrels in proteins. J. Mol. Biol. 177, 567-573 (1984).
12. Koh, E. & Kim, T. Minimal surface as a model of β-sheets. Proteins: Struct. Funct. Bioinf. 61, 559-569 (2005).
13. Lasters, I., Wodak, S. J., Alard, P. & van Cutsem, E. Structural principles of parallel beta-barrels in proteins. Proc. Natl. Acad. Sci. U.S.A. 85, 3338-3342 (1988).
14. Salemme, F. R. Conformational and geometrical properties of beta-sheets in proteins. III. Isotropically stressed configurations. J. Mol. Biol. 146, 143-156 (1981).
15. Lin, Y.-R. et al. Control over overall shape and size in de novo designed proteins. Proc. Natl. Acad. Sci. U.S.A. 112, E5478-85 (2015).
16. Kuhlman, B. et al. Design of a novel globular protein fold with atomic-level accuracy. Science 302, 1364-1368 (2003).
17. Murzin, A. G., Lesk, A. M. & Chothia, C. Principles determining the structure of beta-sheet barrels in proteins. I. A theoretical analysis. J. Mol. Biol. 236, 1369-1381 (1994).
18. Murzin, A. G., Lesk, A. M. & Chothia, C. Principles determining the structure of beta-sheet barrels in proteins. II. The observed structures. J. Mol. Biol. 236, 1382-1400 (1994).
19. McLachlan, A. D. Gene duplications in the structural evolution of chymotrypsin. J. Mol. Biol. 128, 49-79 (1979).
20. Smith, C. K., Withka, J. M. & Regan, L. A Thermodynamic Scale for the .beta.-Sheet Forming Tendencies of the Amino Acids. Biochemistry 33, 5510-5517 (1994).
21. Minor, D. L., Jr & Kim, P. S. Measurement of the beta-sheet-forming propensities of amino acids. Nature 367, 660-663 (1994).
22. Ho, B. K. & Curmi, P. M. G. Twist and shear in β-sheets and β-ribbons. J. Mol. Biol. 317, 291-308 (2002).
23. Fujiwara, K., Ebisawa, S., Watanabe, Y., Toda, H. & Ikeguchi, M. Local sequence of protein β-strands influences twist and bend angles. Proteins: Struct. Funct. Bioinf. 82, 1484-1493 (2014).
24. Hemmingsen, J. M., Gernert, K. M., Richardson, J. S. & Richardson, D. C. The tyrosine corner: A feature of most greek key β-barrel proteins. Protein Sci. 3, 1927-1937 (1994).
25. Paige, J. S., Wu, K. Y. & Jaffrey, S. R. RNA mimics of green fluorescent protein. Science 333, 642-646 (2011).
26. Warner, K. D. et al. Structural basis for activity of highly efficient RNA mimics of green fluorescent protein. Nat. Struct. Mol. Biol. 21, 658-663 (2014).
27. Allison, B. et al. Computational design of protein-small molecule interfaces. J. Struct. Biol. 185, 193-202 (2014).
28. Zanghellini, A. et al. New algorithms and an in silico benchmark for computational enzyme design. Protein Sci. 15, 2785-2794 (2006).
29. Rocklin, G. J. et al. Global analysis of protein folding using massively parallel design, synthesis, and testing. Science 357, 168-175 (2017).
30. Rubin, A. F. et al. A statistical framework for analyzing deep mutational scanning data. Genome Biol. 18, 150 (2017).
31. Meech, S. R. Excited state reactions in fluorescent proteins. Chem. Soc. Rev. 38, 2922 (2009).
32. Merkel, J. S. & Regan, L. Aromatic rescue of glycine in beta sheets. Fold. Des. 3, 449-455 (1998).
33. Conway, P., Tyka, M. D., DiMaio, F., Konerding, D. E. & Baker, D. Relaxation of backbone bond geometry improves protein energy landscape modeling. Protein Sci. 23, 47-55 (2014).
34. Hauser, C. A. E. et al. Natural tri- to hexapeptides self-assemble in water to amyloid-type fiber aggregates by unexpected-helical intermediate structures. Proceedings of the National Academy of Sciences 108, 1361-1366 (2011).
35. Gront, D., Kmiecik, S. & Kolinski, A. Backbone building from quadrilaterals: a fast and accurate algorithm for protein backbone reconstruction from alpha carbon coordinates. J. Comput. Chem. 28, 1593-1597 (2007).
36. Huang, P.-S. et al. RosettaRemodel: a generalized framework for flexible backbone protein design. PLoS One 6, e24109 (2011).
37. Davis, I. W. & Baker, D. RosettaLigand docking with full ligand and receptor flexibility. J. Mol. Biol. 385, 381-392 (2009).
38. Park, H. et al. Simultaneous Optimization of Biomolecular Energy Functions on Features from Small Molecules and Macromolecules. J. Chem. Theory Comput. 12, 6201-6212 (2016).
39. Procko, E. et al. Computational design of a protein-based enzyme inhibitor. J. Mol. Biol. 425, 3563-3575 (2013).
40. Thyme, S. B. et al. Reprogramming homing endonuclease specificity through computational design and directed evolution. Nucleic Acids Res. 42, 2564-2576 (2014).
41. Chao, G. et al. Isolating and engineering human antibodies using yeast surface display. Nat. Protoc. 1, 755-768 (2006).
42. Whitehead, T. A. et al. Optimization of affinity, specificity and function of designed influenza inhibitors using deep sequencing. Nat. Biotechnol. 30, 543-548 (2012).
43. Zhang, J., Kobert, K., Flouri, T. & Stamatakis, A. PEAR: a fast and accurate Illumina Paired-End reAd mergeR. Bioinformatics 30, 614-620 (2014).
44. Fowler, D. M., Araya, C. L., Gerard, W. & Fields, S. Enrich: software for analysis of protein function by enrichment and depletion of variants. Bioinformatics 27, 3430-3431 (2011).
45. Winter, G. xia2: an expert system for macromolecular crystallography data reduction. J. Appl. Crystallogr. 43, 186-190 (2009).
46. McCoy, A. J. et al. Phasercrystallographic software. J. Appl. Crystallogr. 40, 658-674 (2007).
47. Adams, P. D. et al. PHENIX: a comprehensive Python-based system for macromolecular structure solution. Acta Crystallogr. D Biol. Crystallogr. 66, 213-221 (2010).
48. Emsley, P., Lohkamp, B., Scott, W. G. & Cowtan, K. Features and development of Coot. Acta Crystallogr. D Biol. Crystallogr. 66, 486-501 (2010).
49. Afonine, P. V. et al. Towards automated crystallographic structure refinement withphenix.refine. Acta Crystallogr. D Biol. Crystallogr. 68, 352-367 (2012).
50. Otwinowski, Z. & Minor, W. [20] Processing of X-ray diffraction data collected in oscillation mode. Methods Enzymol. 276, 307-326 (1997).
51. McCoy, A. J. et al. Phaser crystallographic software. J. Appl. Crystallogr. 40, 658-674 (2007).
52. Emsley, P., Lohkamp, B., Scott, W. G. & Cowtan, K. Features and development of Coot. Acta Crystallogr. D Biol. Crystallogr. 66, 486-501 (2010).
53. Afonine, P. V. et al. Towards automated crystallographic structure refinement with phenix.refine. Acta Crystallogr. D Biol. Crystallogr 68, 352-367 (2012).

### Methods

Computational design of nonfunctional β-barrels. De novo design of nonfunctional β-barrels can be divided into two main steps: backbone construction and sequence design. For backbone construction, two different approaches were presented: parametric backbone generation and fragment-based backbone assembly.

Parametric backbone generation and sequence design based on hyperboloid models. β-strand arrangements were generated using the equation of a hyperboloid of revolution with an elliptic cross-section, sampling the elliptic radii around the ideal value of β-barrel radius with n number of strands and a sheer number S. Eight β-strands were arranged as equally spaced straight lines running along the surface of the hyperboloid. A reference Ca was defined as the intersection between the first strand and the cross-section ellipse. The other Ca were systematically populated along the 8 strands from this reference residue. The peptide backbone was generated from the Ca coordinates using the BBQ™ software³⁵. The arrangements of discrete β-strands were minimized with geometric constraints to favor backbone hydrogen bonds. One round of fixed-backbone sequence design calculation was carried out to pack the barrel cavity with hydrophobic residues. The resulting β-strand arrangements with the best hydrogen bond connectivity and the tightest hydrophobic packing were selected to be connected by short (2 to 4 residues) β-turns. Two iterations of the loop hashing protocol implemented in RosettaRemodel™³⁶ were performed to close the strands and refine the turns. The sequence design of those β-turns was constrained to sequence profiles derived from natural proteins. Low energy amino acid sequences were obtained for the connected backbones using a flexible-backbone design protocol. Designs with high sequence propensity for forming β-strands, reasonable peptide bond geometry, and tight-packed hydrophobic cores are selected for experimental test (see Table 2).

Backbone assembly from fragments guided by a 2D map. The presented 2D map (FIG. 1d) was designed with the longest strand length observed in soluble β-barrel structures to obtain a β-barrel tall enough for accommodating a hydrophobic core and a binding cavity. The length of each strand depends on its specific position and the sheer number of the barrel. Glycine-kinks and β-bulges were placed on the map as described in the main text. Specific β-turn types were used to connect the β-strands based on their relative positions to β-bulges. According to the 2D map, we then generated a constraint file and a blueprint file to guide the barrel assembly using backbone fragments in the non-redundant fragment library. In the constraint file, each backbone hydrogen bond was described as a set of distance and angle constraints (see FIG. 11a). To connect the first and last strands, a set of defined distance and torsion constraints specific to the tryptophan corner were added to the constraint file. In the blueprint file, a torsion angle bin was attributed to every residue in the peptide chain, according to the Rosetta™ ABEGO nomenclature. After minimizing the assembled backbones using Rosetta™ centroid scoring function with imposed constraints, our protocol output an ensemble of poly-valine β-barrel backbones with defined Gly-kinks, β-bulges, β-turns and the tryptophan corner. The main challenge in building scaffolds with this protocol is to achieve a good balance between the constraints weight, structure diversity and backbone torsion angles. For this work, we solve this problem by performing two additional rounds of sequence design calculation to regularize and prepare scaffolds for designing ligand binding β-barrels (see FIG. 11b-c).

Sequence design of nonfunctional β-barrels. 500 poly-valine backbones with good hydrogen bonds and torsion angles were selected as input for Rosetta™ sequence design. Low energy sequences for the desired β-barrel fold were optimized over several rounds of flexible-backbone sequence design. We employed a genetic algorithm approach to effectively search the sequence space: each parent backbone was used as input to produce 10 designs through individual Monte Carlo searching trajectory. The best ˜10% of the output designs were selected based on the evaluation for total energy, backbone hydrogen bonds, backbone omega and phi/psi torsion angles and hydrophobic packing interactions. The selected models were used as inputs for the next round of design calculation. After 12 rounds of design and selection, no more improvements on the backbone quality metrics were observed (an indication of searching convergence). We then performed a backbone refinement by minimization in Cartesian space and a final round of design calculation (backbone flexibility was limited in torsion space for all the design calculation). The final top designs converged to the offspring of 3 initial backbones, sharing 36% to 99% sequence identity. For every parent backbone, one or two designs with the best hydrophobic packing interactions were selected for experimental characterization. The four designs (BB1-4) share 46% to 72% sequence identity.

Computational design of DFHBI-binding fluorescence-activating β-barrels. De novo design of DFHBI-binding β-barrels consists of three steps: 1) generation of ensembles β-barrel scaffolds (see above), 2) ligand placement by RIF docking and 3) sequence design. 200 input scaffolds were generated in step 1 and used in the following steps (FIG. 11).

Rotamer Interaction Field (RIF) docking. The Rotamer Interaction Field (RIF) docking method performs a simultaneous, high-resolution search of continuous rigid-body docking space as well as a discrete sequence design space. The search is highly optimized for speed and in many cases, including the application presented here, is exhaustive for given scaffold/ligand pair and design criteria. RIF docking comprises two steps. In the first step, ensembles of interacting discrete side chains (referred to as “rotamers”) tailored to the target are generated. Polar rotamers are placed based on hydrogen bond geometry while apolar rotamers are generated via a docking process and filtered by an energy threshold. All the RIF rotamers are stored in ˜0.5 Å sparse binning of the 6 dimensional rigid body space of their backbones, allowing extremely rapid lookup of rotamers that align with a given scaffold position. To facilitate the following docking step, RIF rotamers are further binned at 1.0 Å, 2.0 Å, 4.0 Å, 8.0 Å and 16.0 Å resolutions. In the second step, a set of β-barrel scaffolds is docked into the produced rotamer ensembles, using a hierarchical branch-and-bound search strategy. Starting with the coarsest 16.0 Å resolution, an enumerative search of scaffold positions is performed: the designable scaffold backbone positions are checked against the RIF to determine whether rotamers can be placed with favorable interacting scores. All acceptable scaffold positions (up to a configurable limit, typically 10 million) are ranked and promoted to the next search stage. Each promoted scaffold is split into 2⁶ child positions in the 6D rigid body space, providing a finer sampling. The search is iterated at 8.0 Å, 4.0 Å, 2.0 Å, 1.0 Å and 0.5 Å resolutions. A final Monte Carlo-based rotamer packing step is performed on the best 10% of rotamer placements to find compatible combinations.

Sequence design of DFHBI-binding β-barrels. A total number of 2,102 DFHBI-scaffold pairs from RIF docking were continued for Rosetta sequence design. Our design protocol iterated between a fixed-backbone binding site design calculation and a flexible-backbone design for the rest of scaffold positions. Three variations of this design protocol were used during the sequence optimization. In the initial two rounds of design calculation, RIF rotamers were fixed to maintain the desired ligand coordination. This fixation was released in the final round of design calculation when binding sites were optimized to some degree. A Rosetta mover that biases aromatic residues for hydrophobic packing were added to the design step after the first round of design. A similar selection approach and Cartesian minimization as described for nonfunctional sequence design were used to propagate sequence search and refine the design models. Evaluations on ligand binding interface energy and shape complementarity were added to the selection criteria. The final set of designs were naturally separated into clusters based on their original RIF docking solutions. For each cluster, a sequence profile was generated to guide an additional two rounds of profile-guided sequence design. 42 designs from 22 RIF docking solutions (20 input scaffolds) were selected for experimental characterization).

Post-design model validation and ligand docking simulation. To validate the protein and ligand conformations of the selected designs, we applied model refinement followed by ligand docking simulation. Protein model refinement was carried out on the unbound model of the designs by running five independent 10-ns MD simulations followed by structural averaging and geometric regularization⁵. Then ligand docking simulation was performed on this refined unbound structure using RosettaLigand™³⁷ using Rosetta™ energy function³⁸, allowing rigid body orientation and intra-molecular conformation of the ligand as well as surrounding protein residues (both on side chains and backbones) to be sampled. The ligand-binding energy landscapes were generated by repeating 2,000 independent docking simulations.

Design of disulfide bonds. The disulfide bonds were designed between the N-terminal 3-10 helix and a residue on one of the β-strands on the opposite side of the tryptophan corner. The first 6 residues of the designs were rebuilt with RosettaRemodel™³⁶ and checked for disulfide bond formation using geometric criteria. Once a disulfide bond was successfully placed, the N-terminal helix was redesigned.

Redesign of β-turns for b11. Three β-turns (loop 3, 5 and 7) surrounding the DFHBI-binding site of b11 were redesigned to make additional protein-ligand contacts. A set of “pre-organized” loops with high content of intra-loop hydrogen bonds and low B-factors were collected from natural β-barrel structures, and used as search template to build individual loop fragment library. Those custom libraries were used as input for RosettaRemodel™ to build an ensemble of loop insertions for b11 in the presence of bound DFHBI. Two rounds of flexible-backbone design calculation were carried out to optimize ligand interface energy and shape complementarity using sequence profiles to maintain the template backbone hydrogen bonds. Designed loop sequences were validated in silico by kinematic loop closure (KIC). 500 loop conformations were generated by independent KIC sampling and scored by Rosetta energy function. 36 designs with improved ligand interface energy, shape complementarity and converged loop sampling were selected for experimental characterization.

Redesign of β-barrel core and DFHBI-binding site for b11L5F.1. After releasing the disulfide bond in b11L5F, with ligand modeled in the lowest-energy docked conformation for b11L5F, we performed another round of design calculation to further optimize the β-barrel core packing and ligand binding interactions. The design protocol was very similar to the one used before with fixed ligand hydrogen-bonding residues from RIF docking. 5 designs with 9-15 mutations after manual inspection were selected for experimental characterization.

Protein expression and purification. Genes encoding the nonfunctional β-barrel designs (41 from parametric design and 4 from fragment-base design) were synthesized and cloned into the pET-29 vector (GenScript, Inc). Plasmids were then transformed into BL21*(DE3) E. coli strain (NEB, Inc). Protein expression was induced either by 1 mM isopropyl β-d-thiogalactopyranoside (IPTG) at 18° C., or by overnight 37° C. growth in Studier autoinduction medium. Cells were lysed either by sonication (for 0.5-1L cultures) or FastPrep™ (MPBio, Inc) (for 5-50 mL cultures). Soluble designs were purified by Ni-NTA affinity resin (Qiagen, Inc) and monomeric species were further separated by Akta Pure™ fast protein liquid chromatography (FPLC)(GE Healthcare, Inc) using a Superdex™ 75 increase 10/300 GL column (GE Healthcare, Inc). 56 genes encoding DFHBI-binding designs were synthesized and cloned into pET-28b vector (Gen9, Inc). Protein expression and purification were carried out in the same way.

Circular dichroism (CD). Purified protein samples were prepared at 0.5 mg/ml in 20 mM Tris buffer (150 mM NaCl, pH8.0) or PBS buffer (25 mM phosphate, 150 mM NaCl, pH7.4). Wavelength scans from 195 nm to 260 nm were recorded at 25 degrees Celsius, 75 degrees Celsius, 95 degrees Celsius and cooling back to 25 degrees Celsius. Thermal denaturation was monitored at 220 nm or 226 nm from 25 degrees Celsius to 95 degrees Celsius. Near-UV wavelength scan from 240 nm to 320 nm and tryptophan fluorescence emission were recorded in the absence and presence of 5M guanidinium chloride (GuHCl). Chemical denaturation in GuHCl was monitored by both tryptophan fluorescence and near-UV CD signal at 285 nm. The concentration of the GuHCl stock solution was measured with a refractometer (Spectronic Instruments, Inc). Far-UV CD experiments were performed on an AVIV model 420 CD spectrometer (Aviv Biomedical, Inc). Near-UV CD and tryptophan fluorescence experiments were performed on a Jasco J-1500 CD spectrometer (Jasco, Inc). Protein concentrations were determined by 280 nm absorbance with a NanoDrop™spectrophotometer (ThermoScientific, Inc). Melting temperatures were estimated by smoothing the sparse data with a Savitsky-Golay filter of order 3 and approximating the smoothed data with a cubic spline to compute derivatives. Reported Tₘ values are the inflection points of the melting curves.

Size Exclusion Chromatography with Multi-Angle Light Scattering (SEC-MALS). Protein samples were prepared at 1-3 mg/ml and applied to a Superdex™ 75 10/300 GL column (GE Healthcare) on a LC 1200 Series HPLC machine (Agilent Technologies, Inc) for size-based separation, followed by a miniDAWN™ TREOS detector (Wyatt Technologies, Inc) for light-scattering signals.

Fluorescence binding assay. Protein-activated DFHBI fluorescence signals were measured in 96-well plate format (Corning 3650) on a Synergy neo2 plate reader (BioTek, Inc) with λₑₓ=450 nm or 460 nm and λₑₘ=500 nm or 510 nm. Binding reactions were performed at 2004, total volume in PBS pH7.4 buffer. Protein concentrations were determined by 280 nm absorbance as described above. DFHBI (Lucerna, Inc) were resuspended in DMSO as instructed to make 100 mM stock and diluted in PBS to 0.5-10 μM. Approximate emission spectra were recorded for active designs from 490 to 600 nm.

Library construction. Deep mutational scanning library for b11L5F were constructed by site-directed mutagenesis as described³⁹. 111 PCR reactions were carried out using DNA oligos directed to each position in two 96-well polypropylene plates (USA Scientific, 1402-9700), and products were pooled and purified by gel extraction kit (Qiagen, Inc) for yeast transformation. Combinatorial libraries for b11L5F.1 and b11L5F.2 were assembled using synthesized DNA oligos (Integrated DNA technologies, Inc) as described. Selected positions were synthesized with 1-2% mixed bases to control mutation rate and library size. Full-length assembled genes were amplified and purified for yeast transformation as described⁴¹.

Yeast surface display and fluorescence activated cell sorting (FACS). Transformed yeast cells (strain EBY100)⁴¹ were washed and re-suspended in PBSF (PBS plus 1 g/L of BSA). DFHBI in DMSO stock was diluted in PBSF for labeling yeast cells at various concentrations. PBSF-treated cells were incubated with DFHBI for 30 min to 1 hour at room temperature on a benchtop rotator (Fisher Scientific, Inc). Library selections were conducted using GFP fluorescence channel at 520 nm with 488 nm laser on a SH800 cell sorter (Sony, Inc). Proteolysis treatment and fluorescence labelling were performed in the same way as described²⁹.

Deep sequencing and data analysis. Pooled DNA samples for b11L5F deep mutational scanning library were transformed twice to obtain biological replicates. Two libraries were treated and sorted in a parallel fashion. Yeast cells of naive and selected libraries were lysed and plasmid DNA was extracted as described⁴². Illumina adaptor sequences and unique library barcodes were appended to each library by PCR amplification using population-specific primers. DNA was sequenced in paired-end mode on a MiSeq Sequencer (Illumina, Inc) using a 300-cycle reagent kit (Catalog number: MS-102-3003). Raw reads were first processed using the PEAR program⁴³ and initial counts analysed with scripts adapted from Enrich⁴⁴. Stability scores were modeled using sequencing counts from proteolysis sorts as described²⁹. Unfolded states were modeled without disulfide bonds (Cysteine were replaced by Serine). Function scores were modeled using sequencing counts from DFHBI fluorescence sorts. A simple meta-analysis statistical model with a single random effect was applied to combine two replicates using the framework developed in Enrich2™³⁰.

BB1 crystal structure. BB1 protein was concentrated to 20 mg/ml in an AMICON™ Ultra-15 centrifugation device (Millipore, Inc), and sequentially exchanged into 20 mM Tris pH8.0 buffer. The initial screening for crystallization conditions was carried out in 96-well hanging drop using commercial kits (Hampton Research, Inc & Qiagen, Inc) and a mosquito (TTP LabTech). With additional optimization, BB1 protein crystallized in 0.1 M BIS-Tris pH 5.0 and 2M ammonium sulfate at 25 degrees Celsius by hanging drop vapor diffusion with 2:1 (protein: solution) ratio. Diffraction data for BB1 was collected over 200° with 1° oscillations, 5 s exposures, at the Advanced Light Source (Berkeley, Calif.) beamline 5.0.1 on an ADSC Q315R area detector, at a crystal-to-detector distance of 180 mm. The data was processed in space group P2₁ to 1.63 Å using Xia2⁴⁵. The BB1 design model was used as a search model for molecular replacement using the program Phaser⁴⁶, which produced a weak solution (TFZ 6.5). From this, a nearly complete model was built using the Autobuild module in Phenix⁴⁷. This required the rebuild-in-place function of autobuild to be set to “False”. Iterative rounds of model building in the graphics program Coot⁴⁸ and refinement using Phenix.refine⁴⁹ produced a model covering the complete BB1 sequence.

b10, b11L5F_LGL crystal structure and mFAPs-DFHBI co-crystal structures. b10 was initially tested for crystallization via sparse matrix screens in 96-well sitting drops using a mosquito (TTP LabTech). Crystallization conditions were then optimized in larger 24-well hanging drops. b10 crystallized in 100 mM HEPES pH7.5 and 2.1M Ammonium sulfate at a concentration of 38 mg/mL. The crystal was transferred to a solution containing 0.1 M HEPES pH 7.5 with 3.4 M Ammonium sulfate and flash frozen in liquid nitrogen. Data was collected with a home-source rotating anode on a Saturn 944+ CCD and processed in HKL2000⁵⁰.

b11L5F_LGL was concentrated to 19.6 mg/mL (1.58 mM), incubated at room temperature for 30 minutes with 1 mM TCEP then mixed with an excess of DFHBI (re-suspended in 100% DMSO). b11L5F_M11 complexed with DFHBI was screened via sparse matrix screens in 96-well sitting drops using a mosquito (TTP LabTech) and crystallized in 100 mM Bis-Tris pH6.5 and 45% (v/v) Polypropylene Glycol P 400. The crystal was flash frozen in liquid nitrogen directly from the crystallization drop. Data was collected with a home-source rotating anode on a Saturn 944+ CCD and processed in HKL2000⁵⁰.

mFAP0 and mFAP1 were mixed with excess DFHBI (re-suspended in 100% DMSO), while keeping the final DMSO concentration at less than 1%. The mFAP0 and mFAP1 complexes were then concentrated to approximately 41 mg/mL and 64 mg/mL, respectively, and initially tested for crystallization via sparse matrix screens in 96-well sitting drops using a mosquito (TTP LabTech). Crystallization conditions were then optimized in larger 24-well hanging drops macroseeded with poor quality crystals obtained in sitting drops. mFAP0 complexed with DFHBI crystallized in 200 mM Sodium chloride, 100 mM HEPES pH 7.5 and 25% (w/v) Polyethylene Glycol 3350. The crystal was transferred to the mother liquor plus 2 mM DFHBI and 10% (w/v) Polyethylene Glycol 400 then flash frozen in liquid nitrogen. Data was collected at the Berkeley Center for Structural Biology at the Advanced Light Source (Berkeley, Calif.), on beamline 5.0.2 at a wavelength of 1.0 Å. and processed in HKL2000⁵⁰. mFAP1 complexed with DFHBI crystallized in 100 mM MES pH6.5 and 12% (w/v) Polyethylene Glycol 20,000. The crystal was transferred to the mother liquor plus 2 M DFHBI and 15% glycerol then flash frozen in liquid nitrogen. Data was collected with a home-source rotating anode on a Saturn 944+ CCD and processed in HKL2000⁵⁰.

Structures were solved by Molecular Replacement with Phaser™ via Phenix™^{47,51} using the Rosetta™ design model with appropriate residues cut back to C-alpha and DFHBI removed. The structure was then built and refined using Coot™⁵² and Phenix™⁵³, respectively, until finished.

Confocal image acquisition. Mammalian cell imaging of mFAP1 and mFAP2 was performed in NIH3T3 cells (Flp-In-3T3, Thermo Fisher Scientific, Inc). NIH3T3 cells were cultured in high-glucose DMEM, 4 mM L-glutamine, 10% fetal bovine serum (FBS, Life Technologies, Inc) at 37 degrees Celsius, 5% CO₂. Cells were plated at 4×10⁴ cells/mL in 35 mm glass-bottomed dishes (Matek, Inc) that were coated with poly-D-lysine. Cells were transfected 24 hours after plating with Lipofectamine™ 3000 (Thermo Fisher Scientific, Inc) at a ratio of 3 μL reagent:1 μL DNA, according to manufacturer's instructions, with 1.25 pCDNA5 plasmids of mFAPs or mFAP fusions (1.25 μg mCherry™ plasmid was added to the cytosolic constructs as a transfection control). Right before imaging, cell media was replaced with FluorBrite™ DMEM (Thermo Fisher Scientific, Inc) media supplemented with GlutaMax™ (Thermo Fisher Scientific, Inc) and 10% v/v FBS, and 20 μM DFHBI. Cells were imaged on a heated stage (37 degrees Celsius). A Leica SP8X system was used for confocal microscopy. A white light laser of 488 was used to excite the DFHBI and detected by a HyD detector, over a range of 495-550 nm. All images were taken using a 63× objective with oil, at 1024×1024 resolution. Imaging of E. coli and S. cerevisiae expressing mFAP2, or Aga2p-mFAP2 (respectively) was performed on the same microscopy without the heating stage.

B) pH Responsiveness of De Novo Beta Barrel Proteins

The de novo beta-barrel protein is designed to bind the deprotonated/anionic state of 3,5-difluoro-4-hydroxybenzylidene imidazolinone (DFHBI), which is the predominant state at a neutral pH of 7.0 as well as the human cellular cystosolic pH of approximately 7.0-7.4. Unbound, free DFHBI in liquid buffer has a pKa (acid dissociation constant) of 5.5 (Paige et al., 2011, Supplemental Figure S4, panel B). Below pH 5.5, the protonated/neutral form of DFHBI predominates in buffer. In particular, the 3,5-difluoro-4-hydroxybenzylidene moiety of DFHBI becomes protonated (i.e. undergoing an anionic oxygen atom to hydroxyl group transition) upon acidification. We found that several of the computationally designed de novo beta-barrel proteins bind to both the deprotonated/anionic and protonated/neutral states of DFHBI, as well as the deprotonated/anionic and protonated/neutral states of an analogous compound called 3,5-difluoro-4-hydroxybenzylidene imidazolinone-2-oxime (DFHO). The following text discusses the de novo beta-barrel protein binding to DFHBI, but the same discussion applies to the de novo beta-barrel protein binding to DFHO.

Upon the de novo beta-barrel protein binding to either the deprotonated/anionic or protonated/neutral states of DFHBI, the high-energy planar conformer is stabilized by protein side-chain and protein backbone interactions to the small molecule DFHBI via van der Waals, electrostatic, and hydrogen bond intermolecular interactions. The planar conformer of DFHBI is strongly fluorescent in the visible electromagnetic spectrum compared with off-planar conformers of DFHBI due to electronic p-orbitals overlapping to a greater extent, which increases the delocalization of pi electrons about the conjugated molecule. Particularly, upon absorption of approximately 484 nanometer (nm) light while DFHBI is bound in the pocket of the de novo beta-barrel protein, a deprotonated/anionic DFHBI valence electron is promoted from the vibrational states of the S 0 ground state molecular orbital to the vibrational states of the S 1 excited state molecular orbital. However, the protonated/neutral DFHBI valence electron is promoted from the vibrational states of the S 0 ground state molecular orbital to vibrational states of the S 1 excited state molecular orbital most efficiently upon absorption of approximately 387 nm light, while DFHBI is bound in the pocket of the de novo beta-barrel protein. Upon valence electron excitation, the electron quickly undergoes internal conversion (releasing energy as heat) until it resides in the S 1 excited state molecular orbital. Finally, the high-energy excited state of DFHBI releases energy in the form of a photon (radiative decay) that forms the basis of the detectable fluorescence signal used to monitor the pH of the environment in which the de novo beta-barrel protein and DFHBI molecular complex reside. Importantly, both the deprotonated/anionic and protonated/neutral state emit visible electromagnetic energy at similar wavelengths of approximately 504 nm. Therefore, the de novo beta-barrel protein and DFHBI molecule combined (bound in complex) can in principle be used as a pH detection system of the environment in which they reside.

Both the protein and DFHBI dye are necessary for reliably and accurately monitoring the pH of the environment (i.e. buffer and/or cellular organelle) in which they reside because the protein stabilizes the planar conformer of DFHBI permitting detectable fluorescence and DFHBI responds to environmental pH through a protonation event of the 3,5-difluoro-4-hydroxybenzylidene chemical moiety. Indeed, the de novo beta-barrel protein, as with all proteins, responds to environmental pH through protonation events of the protein side-chains and protein backbone carbonyl and amide groups, and in the case of acidification of the de novo beta-barrel protein weakens binding affinity of DFHBI and decreases protein stability, increasing the variability in pH quantification via decreasing the fluorescence emission intensities. In fact, the DFHBI chromophore environment directly affects the electronic energy level states that are accessible to DFHBI valence electrons. We have demonstrated this by observing both a blue-shifted peak excitation wavelength and a red-shifted peak emission wavelength in various amino acid mutants of the de novo beta-barrel protein. These changes in electronic energy states accessible to DFHBI valence electrons are impacted by different combinations of protein-ligand van der Waals, electrostatic, and hydrogen bond molecular interactions. In principle, these protein-ligand interactions affect such fluorescence properties as the energy gap of S 1 and S 0 electronic molecular orbitals, the Stokes shift, and the anti-Stokes shift, as well as emergent properties of the bulk system such as the peak excitation wavelength, peak emission wavelength, and quantum yield (by stabilizing the planar conformer of DFHBI). Additionally, the pKa of DFHBI (i.e. the propensity of the 3,5-difluoro-4-hydroxybenzylidene moiety to protonate at a certain pH) can be altered by protein-ligand interactions provided by the de novo beta-barrel protein. Therefore, the primary amino acid sequence of the de novo beta-barrel protein is paramount to conferring fluorescent pH biosensor properties to the system, and is not simply a property of the protonation state of the small molecule DFHBI. Consequently, due to the protein-ligand interactions affecting the aforementioned fluorescence properties of the system, and because a given amino acid sequence encoding a de novo beta-barrel protein might preclude binding of the protonated/neutral DFHBI while permitting binding to the deprotonated/anionic DFHBI (or visa-versa), there are indeed primary amino acid sequences that encode for the de novo beta-barrel protein fold that do not permit use as a fluorescent pH biosensor.

The overall concept that computationally designed de novo beta-barrel proteins in complex with DFHBI can detect the pH (i.e. hydrogen ion concentration) of the environment is founded upon the fact that the fluorescence emission from the protonated/neutral DFHBI increases as pH decreases and the fluorescence emission from the deprotonated/anionic DFHBI decreases as pH decreases. The congruence in peak emission wavelength (i.e. 504 nm) of both the deprotonated/anionic and protonated/neutral DFHBI is a convenient attribute for researchers, but not a requirement for pH detection. Due to the nearly 100 nm blue-shifted peak excitation wavelength of the protonated/neutral DFHBI compared with the deprotonated/anionic DFHBI, the pH of the buffers and/or cellular organelles in which they reside can reliably be monitored using a fluorescence plate reader, fluorimeter, confocal fluorescence microscope or similar device that acquires fluorescence emissions upon sample illumination by calculating the ratio of fluorescence emission intensity emitted at 504 nm upon excitation with 387 nm incident light and 484 nm incident light (chronologically in either order), which was called R_{387nm/484nm}. Interestingly, this emission ratio is independent of protein-ligand complex concentration, which provides a convenient, internally normalized (for concentration) tool for researchers studying the pH of various environments. The pH can be calculated from a simple look-up table obtained from in vitro measurements of R_{387nm/484nm} versus known pH. This discrete look-up table can in principle be fit to an appropriate continuous mathematical function that minimizes the error between the data and the fit, such as an exponential function. This methodology was established using the SV-27 variant of the de novo beta-barrel protein and DFHBI, which yielded the exponential equation:

R_{387nm/484nm}=365.496·e^{(-1.368·pH)}

The methodology for converting two fluorescence microscopy images originating from dual excitation (i.e. excitation at 387 nm laser light and measuring emission at 504 nm creating the first image, and excitation at 484 nm laser light and measuring emission at 504 nm creating the second image) involves calculating R_{387nm/484nm} for each pixel at identical coordinates in x and y dimensions in the two images resulting in a hybrid image, and subsequently using the aforementioned equation to calculate the pH for each pixel in the hybrid image. The resulting pH values at each pixel can then be pseudo-colored using any arbitrary color scale or grey scale, providing a pseudo-colored image portraying spatially accurate pH values. The same methodology can be applied in real-time or post-production to a series of chronologically acquired images producing a movie representing pH fluxes in living cells or environments with high spatiotemporal resolution.

### REFERENCES

1. Paige, J. S., Wu, K., & Jaffrey, S. R. (2011). RNA mimics of green fluorescent protein. Science (New York, N.Y.), 333 (6042), 642-646. doi.org/10.1126/science.1207339
2. Gero Miesenböck, Dino A. De Angelis, & James E. Rothman. (1998). Visualizing secretion and synaptic transmission with pH-sensitive green fluorescent proteins. Nature, 394:192-195. doi:10.1038/28190.

Amino acid sequence variants encoding de novo βbarrel protein folds that confer varying degrees of fluorescence brightness (i.e. fluorescence intensity) are provided herein. The fluorescence brightness of the deprotonated state of the chromophore (i.e. DFHBI or DFHO) bound in the pocket of a de novo βbarrel protein is a product of its quantum yield and extinction coefficient at the peak absorption wavelength of the chromophore bound in the protein pocket. Therefore, variations in fluorescence brightness also imply that modifications to quantum yield and extinction coefficient at the peak absorption wavelength of DFHBI (i.e. 484 nanometers is the peak absorption wavelength of electromagnetic radiation for DFHBI) are also provided by the invention. In particular, the brightest de novo βbarrel protein variant provided, called “mFAP2b”, has the following primary amino acid sequence:

(SEQ ID NO: 69)
MSRAAQLLPGTWQVTMTNEDGQTSQGQWHFQPRSP
YTMDIVAQGTISDGRPIVGYGKATVKTPDTLDIDI
TYPSLGNIKAQGQITMDSPTQFKWDATTKGENDF
HGRLTGTLQRQE*
(Note:
“*” denotes a stop codon).

Fluorescence data acquired on a fluorescence plate reader showed several de novo (barrel protein sequence variants at 10-fold higher concentration than DFHBI, where DFHBI concentration was higher than the dissociation constant of DFHBI for mFAP2 (i.e. approximately 200 nM dissociation constant). mFAP2 is a variant with dimmer fluorescence than mFAP2b, thus the dissociation of DFHBI for mFAP2b is predicted to be identical or lower (i.e. higher affinity for DFHBI) than the dissociation of DFHBI for mFAP2 due to the improved fluorescence brightness of mFAP2b over mFAP2. Under these conditions it can be assumed that every DFHBI in solution was bound in the pocket of a de novo βbarrel protein. Therefore, relative fluorescence intensities emitted at the peak emission wavelengths by each sample can be directly compared for brightness. In excitation spectra plots, mFAP2b showed approximately 4.5 brighter fluorescence than mFAP1 at the peak emission wavelength of DFHBI in these proteins (i.e. 511 nanometers). This indicates that either or both of quantum yield and extinction coefficient at the peak absorption wavelength of DFHBI in these proteins is affected by the primary amino acid sequence and therefore DFHBI chromophore environment. If the de novo β-barrel protein contains the amino acid substitution W27M (tryptophan to methionine at position 27 in the primary amino acid sequence), then the peak emission wavelength was evaluated at 505 nanometers rather than 511 nanometers, since we discovered that a tryptophan at position 27 caused a redshift of approximately 6 nanometers in the peak excitation and peak emission wavelengths. The brightnesses were compared using the same emission wavelength of 525 nanometers, independent of primary amino acid sequence, for direct comparison.

The polypeptides disclosed herein also provides de novo β-barrel protein primary amino acid sequence variants that confer varying brightnesses of the protonated state of DFHBI when bound in the protein pocket. For example, in excitation spectra plots (data not shown), the protonated DFHBI brightness varied according to primary amino acid sequence. Similar to the aforementioned deprotonated state, the fluorescence brightness of the protonated state of the chromophore (i.e. DFHBI or DFHO) bound in the pocket of a de novo β-barrel protein is a product of its quantum yield and extinction coefficient at its peak absorption wavelength (approximately 387 nanometers for the protonated state of DFHBI bound in the protein pocket). The brightnesses of the protonated state of DFHBI for each sample were compared using the peak emission wavelength of the protonated state of DFHBI in the protein pocket, 501 nanometers. Background fluorescence of the buffer (phosphatecitrate buffer with 150 mM NaCl) subtracted from each measurement at each excitation wavelength for the indicated pH. Error bars represent standard deviation of the mean of triplicate conditions.

Engineering of mFAP2 for Higher Stability, Binding Affinity and Brightness

We first sought to improve the stability of mFAPs at low pH, the binding affinity to the phenolic and phenolate forms of DFHBI, as well as the brightness of both complexes. mFAP2 was chosen for optimization because it demonstrated the highest fluorescence in the DFHBI-bound state (absolute quantum yield of 2.1%) and higher affinity (K_{d} of ˜180 nM) compared to mFAP1. The mFAP2 peptide has an insertion in loop 7 that contributes to the higher binding affinity and predicted as relatively flexible by our loop modeling computational protocol. The relative fluorescence of the peptides described below in the presence of chromophore and the estimated binding affinity are given is Table 3A-C below.

TABLE 3A

| | 10 μM | 10 μM | 10 μM | |
|---|---|---|---|---|
| | DFHBI | DFHBI-1T | DFHO | Densitometry |
| mFAP2b | 1.0 ± 0.0 | 0.01 ± 0.0 | 0.01 ± 0.0 | 0.4858 |
| mFAP4 | 0.89 ± 0.13 | 0.14 ± 0.01 | 0.02 ± 0.0 | 0.4489 |
| mFAP5 | 0.81 ± 0.13 | 0.04 ± 0.0 | 0.01 ± 0.0 | 0.7636 |
| mFAP2.5 | 0.8 ± 0.03 | 0.01 ± 0.0 | 0.0 ± 0.0 | 0.6405 |
| mFAP2a | 0.77 ± 0.12 | 0.92 ± 0.05 | 0.04 ± 0.0 | 0.3875 |
| mFAP2.4 | 0.69 ± 0.06 | 0.01 ± 0.0 | 0.0 ± 0.0 | 0.7495 |
| mFAP3 | 0.65 ± 0.14 | 0.63 ± 0.07 | 0.1 ± 0.01 | 0.2893 |
| mFAP2.2.9 | 0.64 ± 0.03 | 0.01 ± 0.0 | 0.0 ± 0.0 | 0.9665 |
| mFAP2.5.1 | 0.61 ± 0.02 | 0.01 ± 0.0 | 0.01 ± 0.0 | 0.5421 |
| mFAP7 | 0.52 ± 0.09 | 0.47 ± 0.04 | 0.01 ± 0.0 | 0.2519 |
| mFAP2.2.10 | 0.44 ± 0.04 | 0.01 ± 0.0 | 0.0 ± 0.0 | 0.8843 |
| mFAP2.5.2 | 0.39 ± 0.05 | 0.0 ± 0.0 | 0.0 ± 0.0 | 0.4993 |
| mFAP2.5.3 | 0.36 ± 0.02 | 0.01 ± 0.0 | 0.0 ± 0.0 | 0.4504 |
| mFAP2.5.4 | 0.34 ± 0.01 | 0.05 ± 0.01 | 0.01 ± 0.0 | 0.5709 |
| mFAP2.5.5 | 0.25 ± 0.01 | 0.0 ± 0.0 | 0.0 ± 0.0 | 0.5419 |
| mFAP2.2.2 | 0.23 ± 0.01 | 0.83 ± 0.08 | 0.02 ± 0.0 | 0.825 |
| mFAP2.2.1 | 0.23 ± 0.02 | 0.81 ± 0.06 | 0.02 ± 0.0 | 0.8782 |
| mFAP2bL2 | 0.22 ± 0.03 | 0.0 ± 0.0 | 0.01 ± 0.0 | 0.191 |
| mFAP2bL5 | 0.22 ± 0.02 | 0.0 ± 0.0 | 0.0 ± 0.0 | 0.4088 |
| mFAP2.2.3 | 0.21 ± 0.03 | 0.65 ± 0.06 | 0.01 ± 0.0 | 0.6837 |
| mFPAP2.2.4 | 0.2 ± 0.03 | 0.66 ± 0.04 | 0.01 ± 0.0 | 0.654 |
| mFAP2.2.12 | 0.2 ± 0.03 | 0.61 ± 0.04 | 0.01 ± 0.0 | 0.8731 |
| mFAP2.3 | 0.2 ± 0.03 | 0.67 ± 0.04 | 0.01 ± 0.0 | 0.7958 |
| mFAP2.0.1 | 0.19 ± 0.01 | 0.05 ± 0.01 | 0.0 ± 0.0 | 0.5854 |
| mFAP8 | 0.18 ± 0.02 | 0.0 ± 0.0 | 0.0 ± 0.0 | 0.3208 |
| mFAP2bL4 | 0.18 ± 0.02 | 0.0 ± 0.0 | 0.0 ± 0.0 | 0.3462 |
| mFAP2.2 | 0.17 ± 0.03 | 0.55 ± 0.02 | 0.01 ± 0.0 | 0.4749 |
| mFAP2.2.5 | 0.17 ± 0.03 | 0.61 ± 0.0 | 0.01 ± 0.0 | 0.6397 |
| mFAP2.2.6 | 0.16 ± 0.03 | 0.46 ± 0.03 | 0.01 ± 0.0 | 0.6241 |
| mFAP2.2.13 | 0.15 ± 0.03 | 0.5 ±0.0 | 0.01 ± 0.0 | 0.9546 |
| mFAP6 | 0.15 ± 0.01 | 0.0 ± 0.0 | 0.0 ± 0.0 | 0.6829 |
| mFAP2 | 0.14 ± 0.03 | 0.33 ± 0.04 | 0.01 ± 0.0 | 0.4332 |
| mFAP2.2.7 | 0.14 ± 0.03 | 0.45 ± 0.01 | 0.01 ± 0.0 | 0.6313 |
| mFAP2.2.14 | 0.14 ± 0.03 | 0.28 ± 0.02 | 0.01 ± 0.0 | 0.7266 |
| mFAP1 | 0.13 ± 0.02 | 0.01 ± 0.0 | 0.0 ± 0.0 | 0.4326 |
| mFAP2bL3 | 0.13 ± 0.03 | 0.0 ± 0.0 | 0.0 ± 0.0 | 0.1451 |
| mFAP2.2.15 | 0.13 ± 0.03 | 0.27 ± 0.02 | 0.0 ± 0.0 | 0.7157 |
| mFAP2bL1 | 0.12 ± 0.02 | 0.0 ± 0.0 | 0.0 ± 0.0 | 0.2045 |
| mFAP2c.0 | 0.11 ± 0.03 | 0.33 ± 0.01 | 0.0 ± 0.0 | 0.4216 |
| mFAP pH | 0.11 ± 0.03 | 0.41 ± 0.02 | 0.01 ± 0.0 | 0.2222 |
| mFAP2.2.16 | 0.11 ± 0.02 | 0.29 ± 0.0 | 0.01 ± 0.0 | 0.5464 |
| mFAP2.2.8 | 0.1 ± 0.02 | 0.16 ± 0.03 | 0.0 ± 0.0 | 1.0 |
| mFAP2a.0 | 0.09 ± 0.02 | 0.01 ± 0.0 | 0.0 ± 0.0 | 0.318 |
| mFAP2.1 | 0.07 ± 0.02 | 0.25 ± 0.01 | 0.01 ± 0.0 | 0.4747 |
| mFAP2.2.17 | 0.07 ± 0.02 | 0.12 ± 0.01 | 0.0 ± 0.0 | 0.3296 |
| mFAP0.1 | 0.06 ± 0.0 | 0.1 ± 0.01 | 0.01 ± 0.0 | 0.7885 |
| mFAP0.2 | 0.03 ± 0.0 | 0.08 ± 0.01 | 0.01 ± 0.0 | 0.7152 |
| mFAP2c.1 | 0.01 ± 0.0 | 0.04 ± 0.01 | 0.0 ± 0.0 | 0.2433 |
| mFAP0.3 | 0.0 ± 0.0 | 0.0 ± 0.0 | 0.01 ± 0.0 | 0.7652 |
| mFAP2c.3 | 0.0 ± 0.0 | 0.01 ± 0.0 | 0.0 ± 0.0 | 0.0217 |
| mFAP2c.4 | 0.0 ± 0.0 | 0.0 ± 0.0 | 0.0 ± 0.0 | 0.0 |
| mFAP2a.1 | 0.0 ± 0.0 | 0.0 ± 0.0 | 0.0 ± 0.0 | 0.0092 |
| mFAP2c.8 | 0.0 ± 0.0 | 0.0 ± 0.0 | 0.0 ± 0.0 | 0.0273 |
| mFAP2c.9 | 0.0 ± 0.0 | 0.0 ± 0.0 | 0.0 ± 0.0 | 0.0018 |
| mFAP2c.10 | 0.0 ± 0.0 | 0.0 ± 0.0 | 0.0 ± 0.0 | 0.0013 |
| mFAP2c.11 | 0.0 ± 0.0 | 0.0 ± 0.0 | 0.0 ± 0.0 | 0.0008 |
| mFAP2c.12 | 0.0 ± 0.0 | 0.0 ± 0.0 | 0.0 ± 0.0 | 0.0042 |
| mFAP2c.5 | 0.0 ± 0.0 | 0.0 ± 0.0 | 0.0 ± 0.0 | 0.0008 |
| mFAP2c.13 | 0.0 ± 0.0 | 0.0 ± 0.0 | 0.0 ± 0.0 | 0.0015 |
| mFAP2c.6 | 0.0 ± 0.0 | 0.0 ± 0.0 | 0.0 ± 0.0 | 0.0004 |
| mFAP2c.14 | 0.0 ± 0.0 | 0.0 ± 0.0 | 0.0 ± 0.0 | 0.0013 |
| mFAP0.4 | 0.0 ± 0.0 | 0.0 ± 0.0 | 0.0 ± 0.0 | 0.0667 |

TABLE 3B

| | 100 nM | 100 nM | 100 nM | |
|---|---|---|---|---|
| | DFHBI | DFHBI-1T | DFHO | Densitometry |
| mFAP2a | 1.0 ± 0.0 | 0.59 ± 0.06 | 0.03 ± 0.01 | 0.3875 |
| mFAP3 | 0.96 ± 0.02 | 0.39 ± 0.07 | 0.05 ± 0.02 | 0.2893 |
| mFAP5 | 0.93 ± 0.03 | 0.04 ± 0.01 | 0.01 ± 0.0 | 0.7636 |
| mFAP2.5 | 0.83 ± 0.02 | 0.01 ± 0.0 | 0.01 ± 0.0 | 0.6405 |
| mFAP2b | 0.82 ± 0.02 | 0.01 ± 0.0 | 0.01 ± 0.0 | 0.4858 |
| mFAP4 | 0.81 ± 0.03 | 0.04 ± 0.01 | 0.01 ± 0.0 | 0.4489 |
| mFAP7 | 0.79 ± 0.04 | 0.29 ± 0.07 | 0.01 ± 0.0 | 0.2519 |
| mFAP2.4 | 0.69 ± 0.03 | 0.01 ± 0.0 | 0.01 ± 0.0 | 0.7495 |
| mFAP2.2.9 | 0.69 ± 0.01 | 0.02 ± 0.0 | 0.01 ± 0.0 | 0.9665 |
| mFAP2.2.10 | 0.61 ± 0.01 | 0.01 ± 0.0 | 0.01 ± 0.0 | 0.8843 |
| mFAP2.5.2 | 0.55 ± 0.03 | 0.01 ± 0.0 | 0.01 ± 0.0 | 0.4993 |
| mFAP2.5.1 | 0.4 ± 0.04 | 0.01 ± 0.0 | 0.01 ± 0.0 | 0.5421 |
| mFAP6 | 0.29 ± 0.04 | 0.01 ± 0.0 | 0.01 ± 0.0 | 0.6829 |
| mFAP2.5.4 | 0.22 ± 0.0 | 0.02 ± 0.0 | 0.01 ± 0.0 | 0.5709 |
| mFAP2.0.1 | 0.22 ± 0.0 | 0.04 ± 0.0 | 0.01 ± 0.0 | 0.5854 |
| mFAP2.5.5 | 0.21 ± 0.02 | 0.01 ± 0.0 | 0.01 ± 0.0 | 0.5419 |
| mFAP1 | 0.18 ± 0.0 | 0.01 ± 0.0 | 0.01 ± 0.0 | 0.4326 |
| mFAP2.5.3 | 0.16 ± 0.04 | 0.01 ± 0.0 | 0.01 ± 0.0 | 0.4504 |
| mFAP pH | 0.16 ± 0.0 | 0.2 ± 0.0 | 0.01 ± 0.0 | 0.2222 |
| mFAP2.2.4 | 0.15 ± 0.0 | 0.5 ± 0.02 | 0.02 ± 0.0 | 0.654 |
| mFAP2.3 | 0.15 ± 0.0 | 0.52 ± 0.02 | 0.02 ± 0.0 | 0.7958 |
| mFAP2.2.3 | 0.15 ± 0.0 | 0.43 ± 0.02 | 0.02 ± 0.0 | 0.6837 |
| mFAP2.2.1 | 0.15 ± 0.0 | 0.59 ± 0.0 | 0.02 ± 0.0 | 0.8782 |
| mFAP2.2.2 | 0.15 ± 0.0 | 0.54 ± 0.01 | 0.02 ± 0.0 | 0.825 |
| mFAP2c.0 | 0.14 ± 0.0 | 0.19 ± 0.01 | 0.01 ± 0.0 | 0.4216 |
| mFAP2.2.15 | 0.14 ± 0.0 | 0.3 ± 0.0 | 0.01 ± 0.0 | 0.7157 |
| mFAP2bL5 | 0.14 ± 0.02 | 0.01 ± 0.0 | 0.01 ± 0.0 | 0.4088 |
| mFAP2.2. 5 | 0.14 ± 0.0 | 0.46 ± 0.01 | 0.02 ± 0.0 | 0.6397 |
| mFAP2.2.16 | 0.14 ± 0.0 | 0.34 ± 0.0 | 0.02 ± 0.0 | 0.5464 |
| mFAP2.2.13 | 0.14 ± 0.0 | 0.54 ± 0.0 | 0.03 ± 0.0 | 0.9546 |
| mFAP2.2.17 | 0.14 ± 0.0 | 0.1 ± 0.01 | 0.01 ± 0.0 | 0.3296 |
| mFAP2.2.12 | 0.13 ± 0.0 | 0.47 ± 0.0 | 0.02 ± 0.0 | 0.8731 |
| mFAP2.1 | 0.13 ± 0.0 | 0.34 ± 0.01 | 0.01 ± 0.0 | 0.4747 |
| mFAP2.2 | 0.13 ± 0.0 | 0.33 ± 0.02 | 0.01 ± 0.0 | 0.4749 |
| mFAP2a.0 | 0.13 ± 0.02 | 0.01 ± 0.0 | 0.01 ± 0.0 | 0.318 |
| mFAP2.2.6 | 0.13 ± 0.0 | 0.35 ± 0.01 | 0.02 ± 0.0 | 0.6241 |
| mFAP2.2.14 | 0.13 ± 0.0 | 0.26 ± 0.01 | 0.01 ± 0.0 | 0.7266 |
| mFAP2.2.7 | 0.13 ± 0.0 | 0.41 ± 0.01 | 0.02 ± 0.0 | 0.6313 |
| mFAP2 | 0.12 ± 0.0 | 0.15 ± 0.01 | 0.01 ± 0.0 | 0.4332 |
| mFAP2bL2 | 0.11 ± 0.02 | 0.01 ± 0.0 | 0.01 ± 0.0 | 0.191 |
| mFAP2c.1 | 0.1 ± 0.0 | 0.22 ± 0.01 | 0.01 ± 0.0 | 0.2433 |
| mFAF2.2.8 | 0.1 ± 0.0 | 0.27 ± 0.01 | 0.01 ± 0.0 | 1.0 |
| mFAP2bL1 | 0.07 ± 0.02 | 0.01 ± 0.0 | 0.01 ± 0.0 | 0.2045 |
| mFAP2bL4 | 0.06 ± 0.01 | 0.01 ± 0.0 | 0.01 ± 0.0 | 0.3462 |
| mFAP2bL3 | 0.05 ± 0.01 | 0.01 ± 0.0 | 0.01 ± 0.0 | 0.1451 |
| mFAP8 | 0.04 ± 0.01 | 0.01 ± 0.0 | 0.01 ± 0.0 | 0.3208 |
| mFAP0.1 | 0.02 ± 0.0 | 0.06 ± 0.0 | 0.02 ± 0.0 | 0.7885 |
| mFAP2c.9 | 0.02 ± 0.01 | 0.01 ± 0.0 | 0.001 ± 0.0 | 0.0018 |
| mFAP2a.1 | 0.02 ± 0.0 | 0.01 ± 0.0 | 0.01 ± 0.0 | 0.0092 |
| mFAP2c.8 | 0.02 ± 0.0 | 0.01 ± 0.0 | 0.01 ± 0.0 | 0.0273 |
| mFAP2c.3 | 0.01 ± 0.0 | 0.01 ± 0.0 | 0.01 ± 0.0 | 0.0217 |
| mFAP2c.4 | 0.01 ± 0.0 | 0.01 ± 0.0 | 0.01 ± 0.0 | 0.0 |
| mFAP2c.10 | 0.01 ± 0.0 | 0.01 ± 0.0 | 0.01 ± 0.0 | 0.0013 |
| mFAP0.2 | 0.01 ± 0.0 | 0.04 ± 0.0 | 0.01 ± 0.0 | 0.7152 |
| mFAP2c.13 | 0.01 ± 0.0 | 0.01 ± 0.0 | 0.01 ± 0.0 | 0.0015 |
| mFAP2c.11 | 0.01 ± 0.0 | 0.01 ± 0.0 | 0.01 ± 0.0 | 0.0008 |
| mFAP2c.5 | 0.01 ± 0.0 | 0.01 ± 0.0 | 0.01 ± 0.0 | 0.0008 |
| mFAP2c.14 | 0.0 ± 0.0 | 0.01 ± 0.0 | 0.01 ± 0.0 | 0.0013 |
| mFAP2c.6 | 0.0 ± 0.0 | 0.01 ± 0.0 | 0.01 ± 0.0 | 0.0004 |
| mFAP2c.12 | 0.0 ± 0.0 | 0.01 ± 0.0 | 0.01 ± 0.0 | 0.0042 |
| mFAP0.4 | 0.0 ± 0.0 | 0.01 ± 0.0 | 0.01 ± 0.0 | 0.0667 |
| mFAP0.3 | 0.0 ± 0.0 | 0.01 ± 0.0 | 0.02 ± 0.0 | 0.7652 |

TABLE 3C

| | DFHBI K_{d} (μM) | DFHBI-1T K_{d} (μM) | DFHO K_{d} (μM) |
|---|---|---|---|
| mFAP0.2 | <0.5 (0.0008 ± 0.01) | <0.5 (0.2958 ± 0.03) | <0.5 (0.0003 ± 0.01) |
| mFAP2c.13 | <0.5 (0.0067 ± 0.01) | 79.4976 ± 29.51 | 42.3372 ± 23.82 |
| mFAP2a | 0.0546 ± 0.01 | 3.7039 ± 0.56 | 8.4683 ± 1.99 |
| mFAP0.3 | <0.5 (0.0689 ± 010) | <0.5 (0.0030 ± 0.01) | 2.1082 ± 1.49 |
| mFAP2c.5 | <0.5 (0.0945 ± 0.02) | 1.1874 ± 0.22 | 3.9817 ± 1.54 |
| mFAP3 | 0.1012 ± 0.06 | 2.3442 ± 0.38 | 3.5622 ± 1.81 |
| mFAP2c.11 | <0.5 (0.1117 ± 0.02) | 0.8747 ± 0.12 | 3.9982 ± 1.21 |
| mFAP2.2.13 | <0.5 (0.1153 ± 0.03) | 1.0636 ± 0.18 | 3.7849 ± 3.10 |
| mFAP2c.9 | <0.5 (0.1292 ± 0.02) | 1.6795 ± 0.23 | 6.0611 ± 1.97 |
| mFAP2c.12 | <0.5 (0.1404 ± 0.02) | 0.7317 ± 0.09 | 3.4271 ± 1.32 |
| mFAP2.3 | <0.5 (0.1463 ± 0.02) | 1.5844 ± 0.34 | 10.4115 ± 3.59 |
| mFAP2.2.12 | <0.5 (0.1517 ± 0.02) | 1.9995 ± 3.31 | 5.8276 ± 4.89 |
| mFAP2.2.17 | <0.5 (0.1529 ± 0.02) | 3.1093 ± 0.38 | 4.2661 ± 4.16 |
| mFAP2.2.7 | <0.5 (0.1541 ± 0.03) | 1.3177 ± 0.22 | 0.7445 ± 0.77 |
| mFAP2.2.1 | <0.5 (0.1569 ± 0.03) | 1.6601 ± 0.34 | <0.5 (0.0098 ± 0.01) |
| mFAP2.2.16 | <0.5 (0.1594 ± 0.03) | 3.1061 ± 0.43 | 2.3478 ± 2.35 |
| mFAP2c.10 | <0.5 (0.1630 ± 0.03) | 3.3847 ± 3.87 | 8.9359 ± 2.65 |
| mFAP pH | <0.5 (0.1638 ± 0.04) | 1.5031 ± 0.31 | 10.4730 ± 4.57 |
| mFAP2c.3 | <0.5 (0.1782 ± 0.03) | 1.2777 ± 0.26 | 10.9815 ± 4.99 |
| mFAP2c.4 | <0.5 (0.1945 ± 0.02) | 4.0694 ± 0.45 | 8.5976 ± 1.45 |
| mFAP2.2.2 | <0.5 (0.1957 ± 0.03) | 1.4160 ± 0.28 | 13.2598 ± 7.38 |
| mFAP2c.0 | <0.5 (0.1986 ± 0.03) | 2.3890 ± 0.49 | 10.1867 ± 3.05 |
| mFAP2.2.15 | <0.5 (0.2058 ± 0.03) | 3.2757 ± 0.79 | 4.6730 ± 4.43 |
| mFAP2c.1 | <0.5 (0.2125 ± 0.02) | 0.9018 ± 0.17 | 7.8463 ± 2.69 |
| mFAP2.2.3 | <0.5 (0.2129 ± 0.04) | 1.8521 ± 0.36 | 5.9604 ± 3.54 |
| mFAP2.2.14 | <0.5 (0.2137 ± 0.04) | 3.2697 ± 0.54 | 8.4056 ± 6.83 |
| mFAP2.2.4 | <0.5 (0.2255 ± 0.06) | 1.9117 ± 0.50 | 5.1666 ± 2.90 |
| mFAP2.2.6 | <0.5 (0.2377 ± 0.03) | 2.1332 ± 0.33 | 6.1682 ± 3.07 |
| mFAP2c.8 | <0.5 (0.2742 ± 0.05) | 1.8872 ± 0.38 | 5.2470 ± 1.98 |
| mFAP4 | <0.5 (0.2839 ± 0.08) | 20.4951 ± 2.85 | 12.8162 ± 4.05 |
| mFAP7 | <0.5 (0.3407 ± 0.09) | 3.7127 ± 0.33 | 16.0744 ± 3.93 |
| mFAP2a.1 | <0.5 (0.4256 ± 0.02) | 3.3935 ± 0.45 | 6.6867 ± 1.50 |
| mFAP2c.14 | <0.5 (0.4558 ± 0.77) | 16.3926 ± 13.03 | 55.0423 ± 23.95 |
| mFAP1 | <0.5 (0.4958 ± 0.09) | 10.8048 ± 1.00 | 7.7959 ± 2.89 |
| mFAP5 | 0.8771 ± 0.14 | 48.9042 ± 7.78 | 28.8692 ± 13.17 |
| mFAP2.2.9 | 0.9454 ± 0.05 | 21.6794 ± 3.99 | 1.9498 ± 2.11 |
| mFAP2.2.10 | 1.0351 ± 0.15 | 19.5182 ± 5.39 | <0.5 (0.0016 ± 0.01) |
| mFAP2.5 | 1.1345 ± 0.09 | 52.5356 ± 7.48 | 34.2296 ± 13.69 |
| mFAP2.2 | 1.1470 ± 0.16 | 43.1588 ± 7.01 | 22.7242 ± 9.66 |
| mFAP2 | 1.1470 ± 0.16 | 43.1588 ± 7.01 | 22.7242 ± 9.66 |
| mFAP2.4 | 1.3138 ± 0.10 | 38.7414 ± 5.71 | 32.7303 ± 14.21 |
| mFAP2.5.2 | 1.3596 ± 0.17 | 40.4714 ± 4.26 | 27.7269 ± 5.72 |
| mFAP2bL2 | 1.4072 ± 0.10 | N/D | N/D |
| mFAP2.5.4 | 1.6382 ± 0.14 | 39.1587 ± 4.33 | 23.4552 ± 7.34 |
| mFAP2b | 1.6678 ± 0.08 | 42.2028 ± 6.03 | 9.0088 ± 1.91 |
| mFAP2.2.5 | 1.7235 ± 0.22 | N/D | N/D |
| mFAP2a.0 | 1.8984 ± 0.23 | 43.2453 ± 6.34 | <0.5 (0.0130 ± 0.02) |
| mFAP0.1 | 2.2631 ± 1.37 | 0.8514 ± 0.16 | 23.4738 ± 19.09 |
| mFAP2bL5 | 2.6987 ± 0.15 | N/D | N/D |
| mFAP2.5.1 | 4.4260 ± 0.44 | 71.7430 ± 13.55 | 32.5163 ± 18.50 |
| mFAP2bL1 | 4.5945 ± 0.48 | N/D | N/D |
| mFAP2.5.5 | 5.5264 ± 0.59 | 66.6045 ± 12.29 | 39.1751 ± 13.38 |
| mFAP2bL3 | 5.9286 ± 0.39 | N/D | N/D |
| mFAP2.5.3 | 9.4730 ± 0.72 | 91.8351 ± 20.65 | <0.5 (0.0004 ± 0.01) |
| mFAP2bL4 | 13.0436 ± 0.82 | N/D | N/D |
| mFAP6 | 13.1430 ± 1.71 | <0.5 (0.0023 ± 0.01) | <0.5 (0.0001 ± 0.00) |
| mFAP8 | 58.1233 ± 8.61 | 40.5055 ± 14.87 | 46.4135 ± 31.00 |
| mFAP2c.6 | 150.1431 ± 264.69 | 98.6905 ± 65.59 | 41.6416 ± 8.08 |

Table 3A-C. Brightness and chromophore affinities of selected mFAP variants. Fluorescence brightness measurements at 10 μM (top table) and 100 nM (middle table) chromophore concentrations were normalized from 0 to 1 across all three chromophores (DFHBI, DFHBI-1T, and DFHO), and the normalized values for two protein concentrations tested for each chromophore at each chromophore concentration were averaged and standard deviations of the average computed. Reported values are the normalized averages and standard deviations of the means for each chromophore concentration and each chromophore. Reported relative densitometry values are the relative densitometry values normalized from 0 to 1 and represent how well each design expresses relative to one another in Lemo21(DE3) E. coli cultures. Thermodynamic dissociation constants (bottom table) are obtained by non-linear least squares fitting of chromophore titrations (n=1) to a single binding site isotherm, and values reported with the standard deviation of the fit. Where the obtained K_{d} values are below the protein concentration tested, the K_{d} and standard deviation of the fit are reported in parentheses. Values with “N/D” were not determined.

Guided by the deep mutational scanning map of stability and fluorescence of b11L5F, we constructed three mutational variants of mFAP2 that were expected to improve the stability of the protein while also aiding crystallization (mFAP2(P50T,S52V), mFAP2(S52T), and mFAP2(P50T,S52V,G100D)). Circular dichroism in the absence of DFHBI revealed that one of these variants, mFAP2(P50T,S52V), hereafter renamed mFAP2.1 (SEQ ID NO:40), demonstrated improved stability at pH 2.93. mFAP2.1 also demonstrated higher fluorescence in the presence of DFHBI at pH 3.66, consistent with improved binding of the phenolic form of DFHBI to the stabilized protein.

We sought to further improve fluorescence of the complex at acidic and neutral pH and built a site-directed mutagenesis (SDM) library at 15 positions of mFAP2.1. The mutagenized positions were selected based on their proximity to the DFHBI binding pocket, as well as in order to try to reduce conformational diversity of the insert into loop7. Fluorescence screening of the SDM library at pH 3.66 and pH 7.36 revealed that the most pH-responsive mutant mFAP2.1(T50P), hereafter known as mFAP2.2 (SEQ ID NO:41), demonstrated ˜1.3-fold higher fluorescence ratio fold-change across pH 3.66-7.36 than mFAP2.1 (data not shown).

Two independent combinatorial libraries were further generated from mFAP2.2: one at 5 positions aimed at increasing loop7 rigidity, and another at 8 positions aimed at optimizing hydrophobic packing of residues in the hydrophobic core (peptides mFAP2.2.x with SEQ ID NO:42-57). The brightest variant from the first library (mFAP2.2(A100E, G101N, N102D, T104H), hereafter known as mFAP2.3 (SEQ ID NO:58)) and the brightest variant of the second library (mFAP2.2(M27W, V39I, V57A, F93W), hereafter known as mFAP2.4 (SEQ ID NO:59)) showed an increase in fluorescence of the phenolate form of DFHBI of ˜1.1-fold and ˜3.4-fold at pH 7.36, respectively (data not shown). The mutations producing mFAP2.3 and mFAP2.4 were combined into one scaffold, generating the mFAP2.5 peptide (SEQ ID NO:60). A last mutation (V67I) was identified by screening a combinatorial library of mutations at 7 positions aimed at packing more methyl groups into the hydrophobic core of mFAP2.5 (peptides mFAP2.5.x with SEQ ID NO:61-65). The new peptide (hereafter referred as mFAP2b (SEQ ID NO:69), FIG. 20 a), is ˜1.2-fold brighter than mFAP2.5 and ˜1.3-fold brighter than mFAP2.4 at neutral pH. Despite producing a stronger fluorescence signal in the presence of DFHBI, mFAP2b had ˜14.1-fold weaker affinity for the chromophore than the initial mFAP2 design. Therefore, a final combinatorial library of mutations at 5 positions was generated, aiming to increasing DFHBI affinity while maintaining its brightness by packing both aromatic and aliphatic residues in the core of mFAP2b. The library was screened for fluorescence of the phenolate form of DFHBI at neutral pH in the presence of a relatively low DFHBI concentration (554.88 nM). The mFAP2b(V13A,M15F), hereafter referred to as mFAP2a (SEQ ID NO:66), displayed ˜1.3-fold brighter fluorescence than mFAP2b at low DFHBI concentration.

Modeling of DFHBI (FIG. 20 b) into the binding pocket of mFAP2a and mFAP2b showed that the mutations V13A and M15F resulted in a void in the binding pocket of mFAP2a. It was hypothesized that a commercially-available variant of the DFHBI chromophore with a trifluoromethyl group, DFHBI-1T (FIG. 20 c) [2], could pack into the void without causing steric clashes with nearby amino acid side-chains and could therefore fit into the mFAP2a binding pocket, while DFHBI-1T would result in steric clashes in the mFAP2b pocket. Studying the fluorescence of mFAP2a and mFAP2b in the presence of DFHBI-1T experimentally validated this hypothesis. The mFAP2a/DFHBI complex was approximately as fluorescent as the mFAP2a/DFHBI-1T complex, whereas the mFAP2b/DFHBI complex was almost 70-fold brighter than the mFAP2b/DFHBI-1T complex (FIG. 20 d,e). Using a laser scanning confocal fluorescence microscope to image E. coli expressing either mFAP2a or mFAP2b labeled with either DFHBI or DFHBI-1T, we demonstrate pronounced chromophore selectivity of mFAP2b for DFHBI over DFHBI-1T, and pronounced chromophore promiscuity of mFAP2a for both DFHBI or DFHBI-1T (FIG. 20 f-i). Based on the absolute and relative quantum yields measured for these four complexes, the brightest protein-chromophore combination is mFAP2a with DFHBI-1T, with an absolute quantum yield of 12.9% (˜3.5-fold dimmer than EGFP). The results of the photochemical characterization of the complexes are given in Table 4.

TABLE 4
Photochemical properties of mFAP2a and mFAP2b compared with controls. % bound is calculated based on reported
K_{d} values and final protein and chromophore concentrations for quantum yield measurements. K_{d} values are
obtained by non-linear least squares fits of the 8 technical replicates per chromophore titration in FIG. 20 d,
e. K_{d} error estimates reported are standard deviations of the non-linear least squares fits.

| | | | | Extinction | | Absolute | Relative | Reported | | |
|---|---|---|---|---|---|---|---|---|---|---|
| | λ_{abs} | λₑₓ | λₑₘ | Coefficient | | Quantum | Quantum | Quantum | % | K_{d} |
| | (nm)* | (nm)* | (nm)* | (M⁻¹ · cm⁻¹)^{†} | Brightness^{‡} | Yield^{§} | Yield^{§} | Yield | Bound | (μM) |
| EGFP | — | 488^{#} | 507^{#} | 56,000^{#} | 33,600^{#} | — | — | 0.60^{#} | — | — |
| mFAP2a + | 491 | 491 | 505 | 64,873 | 3,892 | 0.060 | 0.063 | — | 99.9 | 0.15 ± |
| DFHBI | | | | | | | | | | 0.01 |
| mFAP2a + | 492 | 493 | 505 | 75,113 | 9,690 | 0.129 | 0.128 | — | 95.8 | 5.78 ± |
| DFHBI-1T | | | | | (3.5x dimmer | | | | | 0.86 |
| | | | | | than EGFP) | | | | | |
| mFAP2b + | 495 | 495 | 509 | 60,533 | 5,630 | 0.093 | 0.099 | — | 99.1 | 1.83 ± |
| DFHBI | | | | | | | | | | 0.25 |
| mFAP2b + | 430 | 494 | 505 | 37,843 | 189 | 0.005 | 0.003 | — | 95.1 | 10.53 ± |
| DFHBI-1T | | | | | | | | | | 3.08 |
| DFHBI | 418^{†#} | 423^{†} | 489^{†} | 30,100^{†} | — | 0.001^{#} | — | 0.0007^{†} | — | — |
| | | | | 31,935^{#} | | | | | | |
| DFHBI-1T | 422^{†} | 426^{†} | 495^{†} | 35,400^{†} | — | — | — | 0.00098^{†} | — | — |

*λ_{abs} is peak absorbance wavelength, λₑₓ is peak excitation wavelength, and λₑₘ is peak emission wavelength.
^{†}Extinction coefficients are measured from λ_{abs} estimated based on 1 data point.
^{‡}Brightness is calculated as extinction coefficient multiplied by absolute quantum yield.
^{§}Absolute quantum yield is the average of 10 scans measured with an integrating sphere; relative quantum yield is reported using acridine yellow [13] and fluorescein as reference standards.
K Previously reported value [11].
^{#}Previously reported value [1].

The binding/dissociation equilibrium of the chromophore to the beta-barrel makes the system amenable to super-resolution microscopy, in particular localization microscopy. For such application, the binding and subsequent unbinding of chromophores to mFAPs generates a flash of light (i.e. a blink) that can be fit to a 2-dimensional Gaussian function. A super-resolution image can then be reconstructed from super-imposing several thousands of blinks acquired over the temporal dimension [3]. To test the mFAPs in the context of this application, we covalently fused 6×His-tagged mFAP2a or 6×His-tagged mFAP2b to the C-terminus of the de novo helical filament DHF119 (FIG. 21 a) [4] using a flexible glycine-serine linker, and non-covalently bound these fluorescent protein filaments onto Ni-NTA-coated coverslips for fluorescence imaging. The mFAP2a-covered filaments were labeled with DFHBI-1T whereas the mFAP2b-covered filaments were labeled with DFHBI (at concentrations equal to the thermodynamic dissociation constant of the respective peptide/chromophore complex) (FIG. 21 b-g). Intensity profiles of reconstructed images demonstrated that the average full width at half maximum values matched the diameter of fluorescent protein filament at ˜22 nm (FIG. 21 d,g). Labeling the filaments at chromophore concentrations 10-fold below the thermodynamic dissociation constant resulted in a less frequent blinking rate (data not shown).

The chromophore binding/dissociation equilibrium also provides enhanced photostability to the mFAP system compared to GFP, which is subject to unrecoverable photobleaching because of its covalently bound photoadduct. We sought to compare the photostability of mFAP2a and mFAP2b to AcGFP1. Upon continuous wave imaging at 0.885 Hz using a laser-scanning confocal fluorescence microscope of fixed COS-7 cells, we demonstrate that labeling at a higher concentration of chromophore leads to a reduced photobleach rate (improved photostability) compared to labeling at a lower concentration of chromophore (FIG. 21 j,k). Labeling at a chromophore concentration well below the thermodynamic dissociation constant of the chromophore for the mFAP showed improved photostability over AcGFP1 and reduced apparent photobleaching.

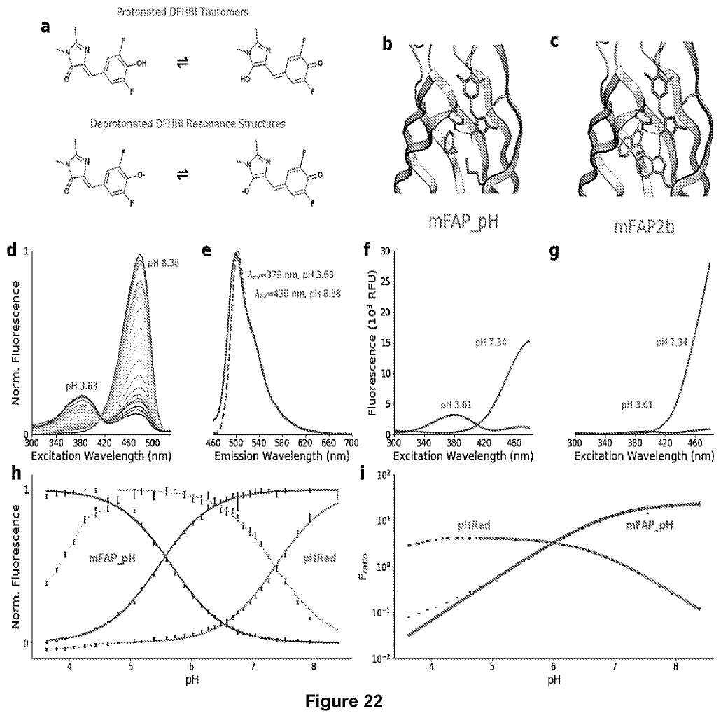

We further sought to engineer the mFAP system into a fluorescent pH-sensor by taking advantage of the pKa of DFHBI (FIG. 22 a). In order to select a variant of mFAP2 that shows a large shift of peak fluorescence excitation wavelength from low to neutral pH, we built another library of variants, based on hypothesis related to binding of the phenolic form of DFHBI (peptides mFAP2c.x with SEQ NO:75-87). The library was screened based on difference in fluorescence excitation spectra at pH 3.61 and pH 7.64. The variant showing the highest fluorescence ratio fold-change across pH 3.61-7.64 was renamed mFAP_pH. Computational modeling suggests that the mutations identified in mFAP_pH compared to mFAP2b (i.e. W27M and W93F) improve the pH-responsiveness of the peptide/chromophore complex by improving binding affinity to both of the tautomers of the phenolic (protonated) form of DFHBI (FIG. 22 b,c). mFAP_pH showed a more marked shift of fluorescent ratio fold-change across pH values and a broader dynamic range of sensitivity across different pH values than the state-of-the-art pHRed™ system (FIG. 21 d-i).

In order to demonstrate that the mFAP system can be used to engineer sensors by fusing peptides into the loops of the beta-barrel surrounding the ligand-binding site, we fused one, two, or four EF-hand motifs into the loop7 of mFAP2a and mFAP2b ((SEQ ID NO:95-120). To do so, the mFAP2b loop7 sequence and five computationally designed loop sequences (peptides mFAP2bL* with SEQ ID NO:70-74) were sampled as linkers for grafting the sequence of one EF-hand motif from PDB ID 1NKF [6] onto loop7 of mFAP2b. To generate this combinatorial linker, we pruned the validated loop7 sequences one residue at a time keeping up to 4 validated residues on the N-terminal or C-terminal linkers relative to the grafted EF-hand motif, optionally adding an additional glycine residue on the N-terminal linker and optionally adding an additional glycine or proline residue on the C-terminal linker. This combinatorial library had a diversity of 1,140 linker designs. The linkers resulting in positively and negatively allosteric Ca²⁺-responsive mFAPs containing one EF-hand motif were combinatorially sampled to act as linkers for grafting two EF-hand motifs from PDB ID 1FW4 [7] onto loop7 of mFAP2b, where the N-terminal helix of PDB ID 1FW4 was truncated up to homologous residues on successfully grafted single EF-hand motif designs. This combinatorial library had a diversity of 385. The linkers resulting in negatively allosteric Ca²⁺-responsive mFAPs containing two EF-hand motifs were combinatorially sampled to act as linkers for grafting four EF-hand motifs from PDB ID 1PRW onto loop7 of mFAP2b [8], where the N-terminal helix of PDB ID 1PRW was truncated up to homologous residues on successfully grafted single EF-hand motif designs. This combinatorial library had a theoretical diversity of 25. We further demonstrated by circular dichroism spectroscopy that calcium binding induces alpha-helical secondary structure formation in a design containing four EF-hand motifs (data not shown).

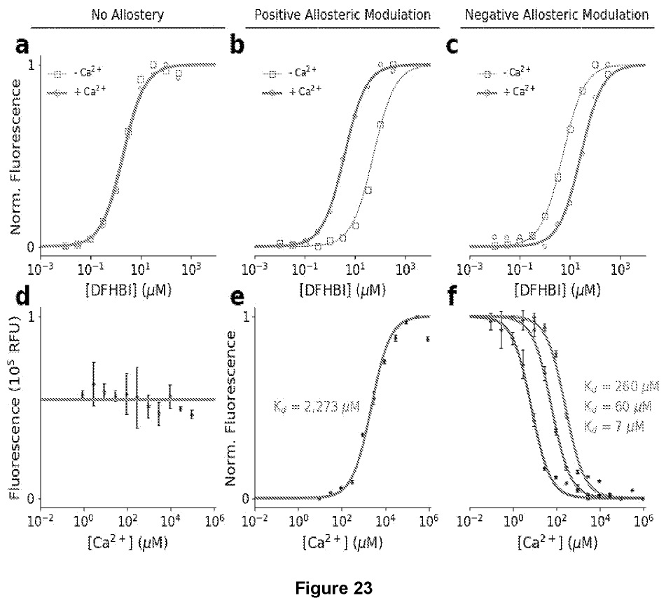

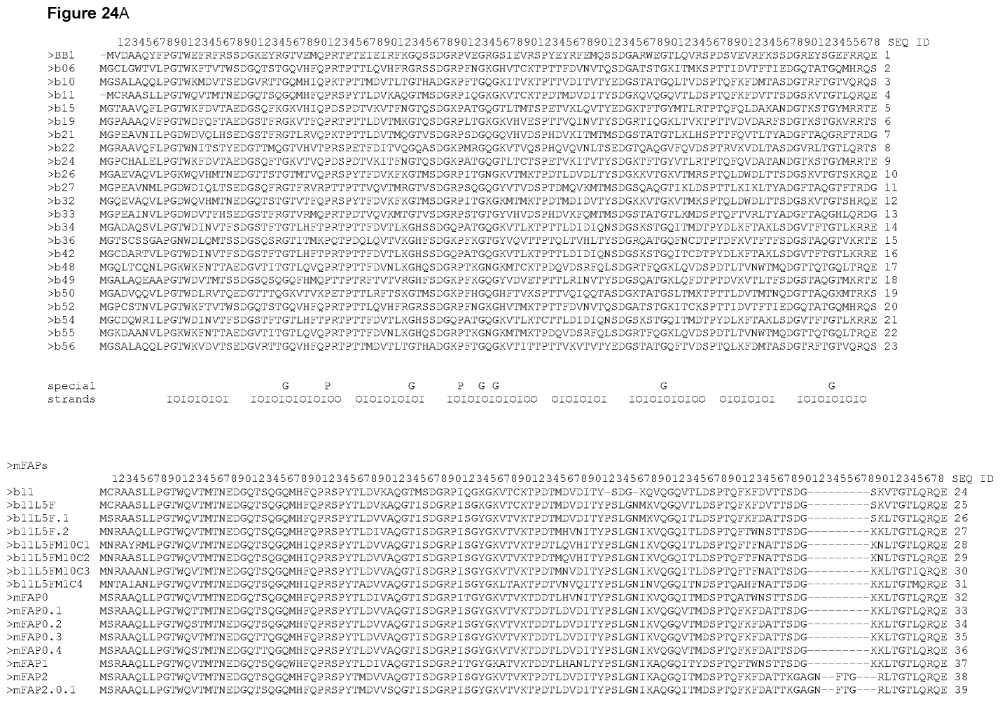

Overall, the resulting peptides exhibited over 100-fold differences in affinity for calcium (Table 5) and both positive and negative allosteric modulation between calcium binding and DFHBI binding (FIG. 23). To show that the allosteric effect is also present in vivo, we expressed negatively allosteric mFAP peptides containing four EF-hand motifs in the cytosol of cultured human HEK293 cells and stimulated Ca²⁺ release into the cytosol by treatment with 100 μM acetylcholine. We demonstrate that indeed Ca²⁺ release into the cytosol results in a marked decrease in fluorescence in HEK293 cells as expected from in vitro Ca²⁺ titration results. An X-ray crystal structure has been solved for EF1p2_mFAP2b (Table 6).

In the present work, optimization of the mFAPs resulted in two peptides (mFAP2a and mFAP2b) highly fluorogenic in the presence of DFHBI and/or DFHBI-1T. We showed that the mFAP is photostable and the chromophore binding/unbinding equilibrium makes the system amenable to use in super-resolution microscopy. We identify mutations that produce a pH-responsive peptide/chromophore pair that can be used for pH sensing. We also show that insertions of calcium binding motifs into a loop of the beta-barrel produce calcium-responsive peptide/chromophore pairs that can be used as sensors. We furthermore propose that the different variants of the mFAP system can be combined in a modular way. For example, the Ca²⁺-responsive mFAP variants presented herein can be used for super-resolution microscopy, in particular localization microscopy, as the fluorescence blinking rate can be tuned by modulating both Ca²⁺ concentration as well as DFHBI concentration. We furthermore propose that, due to the promiscuity of EF-hand motifs for binding Ca²⁺, La³⁺, Tb³⁺, and other ions [10], the Ca²⁺-responsive mFAP variants presented herein can be used to detect Ca²⁺, La³⁺, Tb³⁺, and other ion presences and concentrations, and that these ions can also be used to modulate blinking frequency in super-resolution experiments. Additionally, the mFAP system has the potential of being re-optimized to bind DFHBI-derived and other chromophores with different fluorescence spectra, as shown by the low-level binding of the chromophore DFHO [11] to the peptide mFAP3. mFAPs could be used as modular fluorescent sensors for detection and quantification of other small-molecules, ions, peptides, or nucleic acids by insertion of their respective binding peptides into the loops of the mFAPs.

TABLE 5
Ca²⁺-responsive fluorogenic mFAP variants.

| | DFHBI | DFHBI | Ca²⁺ | Hill | | | (K_{d}⁺ · | Max. \|θ⁺ · |
|---|---|---|---|---|---|---|---|---|
| | K_{d}⁺ | K_{d}⁻ | K_{d} | Coefficient, | K_{d}⁻/ | K_{d}⁺/ | K_{d}⁻)^{1/2} | θ⁻\| |
| Design Name | (μM) | (μM) | (μM) | n_{H} | K_{d}⁺ | K_{d}⁻ | (μM) | (%) |
| EF1p_mFAP2b | 2.8 ± 0.1 | 16.6 ± 1.3 | 4,114.3 ± 639.7 | 1.3 ± 0.2 | 5.8 ± 0.6 | — | 6.9 ± 0.3 | 41.5 |
| EF1p_mFAP2a | 1.1 ± 0.0 | 33.3 ± 3.2 | 352.3 + 11.4 | 0.9 ± 0.0 | 31.2 ± 3.1 | — | 6.0 ± 0.3 | 69.6 |
| EF1p2_mFAP2b | 10.8 ± 1.1 | 34.8 ± 3.3 | 2,273.2 ± 285.3 | 0.9 ± 0.1 | 3.2 ± 0.5 | — | 19.4 ± 1.4 | 28.4 |
| EF1p2_mFAP2a | 1.3 ± 0.1 | 45.0 ± 5.3 | 272.8 ± 3.9 | 1.0 ± 0.0 | 35.5 ± 4.4 | — | 7.6 ± 0.5 | 71.3 |
| EF1p3_mFAP2b | 3.4 ± 0.2 | 27.1 ± 2.2 | 1,720.6 ± 81.1 | 1.0 ± 0.0 | 7.9 ± 0.8 | — | 9.6 ± 0.5 | 47.6 |
| EF1p3_mFAP2a | 1.5 ± 0.1 | 56.2 ± 8.0 | 588.3 ± 13.5 | 0.9 ± 0.0 | 36.5 ± 5.5 | — | 9.3 ± 0.7 | 71.6 |
| EF1n_mFAP2b | 6.9 ± 0.3 | 5.0 ± 0.3 | 259.9 ± 28.7 | 1.2 ± 0.1 | — | 1.4 ± 0.1 | 5.9 ± 0.2 | 8.0 |
| EF1n_mFAP2a | 19.9 ± 1.6 | 2.6 ± 0.1 | 3,017.6 ± 202.9 | 0.9 ± 0.0 | — | 7.5 ± 0.7 | 7.2 ± 0.3 | 46.6 |
| EF1n2_mFAP2b | 42.2 ± 8.0 | 27.6 ± 2.2 | 139.8 ± 14.2 | 1.1 ± 0.1 | — | 1.5 ± 0.3 | 34.1 ± 3.5 | 10.6 |
| EF1n2_mFAP2a | 43.6 ± 4.8 | 4.7 ± 0.3 | 504.4 ± 26.5 | 0.9 ± 0.0 | — | 9.3 ± 1.2 | 14.3 ± 0.9 | 50.7 |
| EF1n3_mFAP2b | 26.9 ± 2.2 | 3.2 ± 0.1 | 125.2 ± 78.0 | 0.8 ± 0.5 | — | 8.4 ± 0.8 | 9.3 ± 0.4 | 48.6 |
| EF1n3_mFAP2a | 5.4 ± 0.2 | 1.3 ± 0.1 | 240.9 ± 13.2 | 1.0 ± 0.0 | — | 4.3 ± 0.3 | 2.6 ± 0.1 | 35.0 |
| EF2n_mFAP2b | 38.0 ± 3.6 | 10.0 ± 0.7 | 41.9 ± 5.9 | 1.5 ± 0.1 | — | 3.8 ± 0.4 | 19.5 ± 1.1 | 32.1 |
| EF2n_mFAP2a | 51.9 ± 14.2 | 2.4 ± 0.1 | 60.0 ± 4.1 | 1.1 ± 0.1 | — | 22.0 ± 6.1 | 11.1 ± 1.6 | 64.8 |
| EF2n2_mFAP2b | 14.5 ± 9.7 | 14.6 ± 2.7 | 161.8 ± 37.8 | 1.3 ± 0.3 | — | 1.0 ± 0.7 | 14.5 ± 5.1 | 0.2 |
| EF2n2_mFAP2a | 45.7 ± 13.0 | 2.5 ± 0.1 | 225.9 ± 15.3 | 1.1 ± 0.1 | — | 18.2 ± 5.2 | 10.7 ± 1.5 | 62.0 |
| EF2n3_mFAP2b | 96.6 ± 25.1 | 27.4 ± 2.3 | 167.3 ± 25.7 | 1.1 ± 0.2 | — | 3.5 ± 1.0 | 51.4 ± 7.0 | 30.5 |
| EF2n3_mFAP2a | 85.7 ± 23.7 | 9.1 ± 0.5 | 191.5 ± 7.1 | 1.0 ± 0.0 | — | 9.4 ± 2.7 | 27.9 ± 4.0 | 50.9 |
| EF4n_mFAP2b | 65.1 ± 10.4* | 28.9 ± 2.6^{†} | 7.0 ± 0.6* | 1.0 ± 0.1 | — | 2.3 ± 0.4 | 43.4 ± 4.0 | 20.1 |
| EF4n_mFAP2a | 69.9 ± 11.8* | 62.7 ± 10.3^{†} | 9.5 ± 1.7* | 0.8 ± 0.1 | — | 1.1 ± 0.3 | 66.2 ± 7.8 | 2.7 |

DFHBI K_{d}⁺(in excess Ca²⁺), DFHBI K_{d}⁻(in absence of Ca²⁺), and Ca²⁺K_{d} (in excess DFHBI) are computed by fitting the normalized fluorescence readouts from titrations to a single binding site isotherm equation with Hill coefficient of 1 using non-linear least squares fitting. Standard deviations of the fit are reported. Hill coefficients are reported for fitting the titration data to a single binding site isotherm with variable Hill coefficient using non-linear least squares fitting, and the standard deviation of the fit are reported. For positive allosteric modulators, K_{d}⁻/K_{d}⁺ is reported, and for negative allosteric modulators, K_{d}⁺/K_{d}⁻ is reported. Computed (K_{d}⁺·K_{d}⁻)^{1/2} values and maximum absolute values of the difference in fraction of sensor bound by DFHBI, Max. |θ⁺−θ⁻|, is reported as a percent. * Chelex 100 was used to pre-treat buffers. † Titrations carried out in EGTA.

TABLE 6
X-ray crystallography data collection and
refinement metrics for EF1p2_mFAP2b.

| Data Collection | | | Refinement | |
|---|---|---|---|---|
| Space group | P 1 21 1 | | R-work | 0.1691 |
| Unit cell | | | R-free | 0.2010 |
| a, b, c | 36.4, 35.6, 86.6 | | Number of non- | 2,160 |
| | | | hydrogen atoms | |
| alpha, beta, | 90, 90.7, 90 | | macromolecules | 1,904 |
| gamma | | | | |
| Wavelength (Å) | 1.54 | | ligands | 38 |
| Resolution | 50-2.1 | (2.2-2.1) | water | 218 |
| range (Å) | | | | |
| Unique | 13,070 | | Protein residues | 254 |
| reflections | | | | |
| R-merge | 0.025 | (0.055) | RMS(bonds) | 0.004 |
| R-meas | 0.030 | (0.068) | RMS(angles) | 0.83 |
| R-pim | 0.016 | (0.040) | Ramachandran | 98.00 |
| | | | favored (%) | |
| CC1/2 | (0.996) | | Ramachandran | 2.00 |
| | | | allowed (%) | |
| I/sigma(I) | 35.2 | (14.7) | Ramachandran | 0.00 |
| | | | outliers (%) | |
| Chi\hat{ }2 | 0.595 | | Clashscore | 4.27 |
| Multiplicity | 3.6 | (2.8) | Average B-factor | 20.11 |
| Completeness (%) | 98.9 | (86.2) | macromolecules | 19.33 |

| | | | |
|---|---|---|---|
| Wilson B-factor | 18.62 | ligands | 14.58 |

| | |
|---|---|
| solvent | 27.9 |

### REFERENCES

1. Dou, J., Vorobieva, A. A., et al. De novo design of a fluorescence-activating β-barrel. Nature 561, 485-491 (2018).
2. Song, W., et al. Plug-and-play fluorophores extend the spectral properties of Spinach. J. Am. Chem. Soc. 136, 1198-1201 (2014).
3. Bozhanova, N. G., et al. Protein labeling for live cell fluorescence microscopy with a highly photostable renewable signal. Chem. Sci. 8, 7138-7142 (2017).
4. Shen, H., Fallas, J. A., et al. De novo design of self-assembling helical protein filaments. Science 362, 705-709 (2018).
5. Tantama, M., et al. Imaging intracellular pH in live cells with a genetically encoded red fluorescent protein sensor. J. Am. Chem. Soc. 133, 10034-10037 (2011).
6. Siedlecka, M., et al. Alpha-helix nucleation by a calcium-binding peptide loop. Proc. Natl. Acad. Sci. U.S.A. 96, 903-908 (1999).
7. Olsson, L. L. & Sjölin, L. Structure of Escherichia coli fragment TR2C from calmodulin to 1.7 A resolution. Acta Crystallogr. D Biol. Crystallogr. 57, 664-669 (2001).
8. Fallon, J. L. & Quiocho, F. A. A closed compact structure of native Ca(2+)-calmodulin. Structure 11, 1303-1307 (2003).
9. Chen, T.-W., et al. Ultra-sensitive fluorescent proteins for imaging neuronal activity. Nature 499, 295-300 (2013).
10. Ye, Y., et al. A grafting approach to obtain site-specific metal-binding properties of EF-hand proteins. Protein Eng. 16, 429-434 (2003).
11. Song, W., Filonov, G. S., et al. Imaging RNA polymerase III transcription using a photostable RNA-fluorophore complex. Nat. Chem. Biol. 13, 1187-1194 (2017).
12. Tebo, A. G. et al. Circularly Permuted Fluorogenic Proteins for the Design of Modular Biosensors. ACS Chem. Biol. 13, 2392-2397 (2018).
13. Olmsted, J. calorimetric determinations of absolute fluorescence quantum yields. The Journal of Physical Chemistry 83, 2581-2584 (1979).

1. A non-naturally occurring beta barrel polypeptide comprising the formula X1-X2-X3-X4-X5-X6-X7-X8-X9-X10-X11-X12-X13-X14-X15-X16-X17-X18-X19-X20, wherein:
X1 comprises a capping domain;
X2 comprises a beta strand,
wherein a contiguous C-terminal portion of X1 and N-terminal portion of X2 comprise the amino acid sequence Z1-P-G-Z2-W, where Z1 and Z2 are any amino acid;
X3 comprises a beta turn;
X4 comprises a beta strand that includes an internal G residue and a P at its C terminus;
X5 comprises a single polar amino acid;
X6 comprises a beta turn;
X7 comprises a beta strand including an internal G residue;
X8 comprises a beta turn;
X9 comprises a beta strand including an internal P residue and 2 internal G residues;
X10 comprises a single polar amino acid;
X11 comprises a beta turn;
X12 comprises a beta strand;
X13 comprises a beta turn;
X14 comprises a beta sheet with an internal G residue;
X15 comprises a single polar amino acid;
X16 comprises a beta turn;
X17 comprises a beta strand;
X18 comprises a beta turn; and
X19 comprises a beta strand.

2.-5. (canceled)

6. The beta barrel polypeptide of claim 1, wherein X1 comprises the amino acid sequence at least 50%, 55%, 60%, 65%, 70%, 75%, 80%, 85%, 90%, 95%, or 100% identical to the amino acid sequence RA(A/I/Y)(R/S/Q/A)LLP (SEQ ID NO: 121) or RAAQLLP (SEQ ID NO: 134), wherein the highlighted residue is invariant.

7. The beta barrel polypeptide of claim 1, wherein X2 comprises the amino acid sequence at least 50%, 55%, 60%, 65%, 70%, 75%, 80%, 85%, 90%, 95%, or 100% identical to the amino acid sequence G (T/K/N/D) WQZT(M/F)TN (SEQ ID NO: 122) wherein Z is any amino acid, or GTWQ(V/L/A/I) T(M/F)TN (SEQ ID NO: 135), wherein the highlighted residues are invariant.

8. The beta barrel polypeptide of claim 1, wherein X3 comprises the amino acid sequence (E/S)DG or EDG, and/or wherein X6 comprises the amino acid sequence (T/S)PZ3, where Z3 is polar amino acid or Tyr; or wherein X6 is SPY.

9. The beta barrel polypeptide of claim 1, wherein X4 comprises the amino acid sequence at least 50%, 55%, 60%, 65%, 70%, 75%, 80%, 85%, 90%, 95%, or 100% identical to the amino acid sequence QTSQGQMHFQP (SEQ ID NO: 123), wherein the highlighted residues are invariant.

10.-11. (canceled)

12. The beta barrel polypeptide of claim 1, wherein X7 comprises the amino acid sequence at least 50%, 55%, 60%, 65%, 70%, 75%, 80%, 85%, 90%, 95%, or 100% identical to the amino acid sequence T(L/A/M)D(I/V)(K/V)(A/S) GT(I/M) (SEQ ID NO: 124) or TMDIVAQGTI (SEQ ID NO: 136), wherein the highlighted residues are invariant.

13. The beta barrel polypeptide of claim 1, wherein X8 comprises the amino acid sequence (S/A)DG or SDG.

14. The beta barrel polypeptide of claim 1, wherein X9 comprises the amino acid sequence at least 50%, 55%, 60%, 65%, 70%, 75%, 80%, 85%, 90%, 95%, or 100% identical to the amino acid sequence RPI(Q/S/T/V)G(Y/K)GK(L/V/A)T(V/C/A) (SEQ ID NO: 125) or RPIVGYGKATV (SEQ ID NO:137), wherein the highlighted residues are invariant.

15. (canceled)

16. The beta barrel polypeptide of claim 1, wherein X11 comprises the amino acid sequence (S/T)(P/C)(polar or Y), or wherein X 11 is TPD.

17. The beta barrel polypeptide of claim 1, wherein X12 comprises the amino acid sequence at least 50%, 55%, 60%, 65%, 70%, 75%, 80%, 85%, 90%, 95%, or 100% identical to the amino acid sequence T(M/L/V)(D/H/Q/N)(V/A/L/I)(D/N/H/Q)(I/L/V) T(Y/W) (SEQ ID NO: 126) or TLDIDITY (SEQ ID NO:138).

18. The beta barrel polypeptide of claim 1, wherein X13 comprises the amino acid sequence (S/E)DG, or wherein X13 comprises the amino acid sequence at least 60%, 80%, or 100% identical to PSLGN (SEQ ID NO: 127).

19. The beta barrel polypeptide of claim 1, wherein X14 comprises the amino acid sequence at least 50%, 55%, 60%, 65%, 70%, 75%, 80%, 85%, 90%, 95%, or 100% identical to the amino acid sequence (K/M/I/L)(Q/K)(V/A/G)QGQ(V/I)T(M/L/Y) (SEQ ID NO: 128) or IKAQGQITM (SEQ ID NO: 139), wherein the highlighted residues are invariant.

20. (canceled)

21. The beta barrel polypeptide of claim 1, wherein X16 comprises the amino acid sequence (S/T)P(D/T/Y), or wherein X16 comprises the amino acid sequence SPT.

22. The beta barrel polypeptide of claim 1, wherein X17 comprises the amino acid sequence at least 50%, 55%, 60%, 65%, 70%, 75%, 80%, 85%, 90%, 95%, or 100% identical to the amino acid sequence Q(F/A)(K/T/H)(F/W)(D/N)(V/A/S/G)(T/Q/H/E) (T/F/V/Y) (SEQ ID NO: 129) or QFKFDATT (SEQ ID NO: 140).

23. The beta barrel polypeptide of claim 1, wherein X19 comprises the amino acid sequence at least 50%, 55%, 60%, 65%, 70%, 75%, 80%, 85%, 90%, 95%, or 100% identical to the amino acid sequence [(S/K/N/H)](K/R/I/N)(V/L)TGT(L/I/M)QRQE (SEQ ID NO: 132) or RLTGTLQRQE (SEQ ID NO: 144), wherein residues in brackets are optional.

24. The beta barrel polypeptide of claim 1, wherein X18 comprises the amino acid sequence selected from the group consisting of (S/E/N/A/Q)DG, SDG, K(G/Q/K/T)(A/D/E/N)(G/D/N)(N/G/D/Y/S) (SEQ ID NO: 130), KG(A/D/E)(G/D/N)(N/G/D/Y) (SEQ ID NO: 131), KGENDFHG (SEQ ID NO:141), KGADGWHG (SEQ ID NO: 142), and KGAGNFTG (SEQ ID NO: 143).

25.-27. (canceled)

28. The beta barrel polypeptide of claim 1, comprising the amino acid sequence at least 50%, 55%, 60%, 65%, 70%, 75%, 80%, 85%, 90%, 95%, or 100% identical to the amino acid sequence selected from SEQ ID NOS: 1-120.

29.-30. (canceled)

31. The polypeptide of claim 28, wherein the polypeptide comprises residues at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or all 45 of the following positions relative to SEQ ID NO: 38 (mFAP2), with numbering starting from the first residue after the optional N-terminal methionine residue:

| Position (mFAP2 numbering, no M) | Residues |
|---|---|
| 13 | V, A, L, I |
| 15 | M, F |
| 17 | N |
| 23 | S, T |
| 27 | W, M |
| 29 | F, I |
| 37 | M, L |
| 39 | I, V |
| 41 | A |
| 45 | I, M, L |
| 49 | R |
| 50 | P, T |
| 51 | I |
| 52 | V, S, Q, T |
| 57 | A, V, L |
| 59 | V, A, C |
| 65 | L, M, V |
| 67 | I, V, A |
| 69 | I, L |
| 71 | Y, W |
| 72-76 | PSLGN (SEQ ID |
| | NO: 127) |
| 77 | I, M, L |
| 79 | A, G, V |
| 83 | I, V |
| 85 | M, L, Y, N |
| 91 | F, A |
| 93 | W, F |
| 95 | A, G, S |
| 97 | T |
| 98-100 | KG (E/A) |
| 101-103 | (N/G/D) (D/N/G) F |
| 104-106 | (H/T/Q) GR |
| 105 | F, W, Y |
| 107 | L, V |
| 111 | L, I, M |

32. A nucleic acid encoding the beta barrel polypeptide of claim 1.

33.-35. (canceled)

36. Use of the beta barrel polypeptide of claim 1 for uses including, but not limited to pH sensing, ion-sensing/detection (including but not limited to Ca²⁺, La³⁺, Tb³⁺, and other ion sensing/detection/quantification), super-resolution microscopy, localization microscopy, and detection and quantification of other small-molecules, ions, peptides, or nucleic acids by insertion of their respective binding peptides into the loops of the polypeptides.

37. (canceled)


SEQUENCE LISTING (144 sequences)

SEQ ID NO: 1 - protein, 110 aa, Artificial Sequence
  Synthetic
  MISC_FEATURE (1)..(1): optionally absent
MVDAAQYFPGTWEFRFRSSDGKEYRGTVEMQPRTPTEIEIRFKGQSSDGRPVEGRGSIEVRSPYEYRFEMQSSDGARWEGTLQVRSPDSVEVRFKSSDGREYSGEFRRQE

SEQ ID NO: 2 - protein, 111 aa, Artificial Sequence
  Synthetic
  MISC_FEATURE (1)..(1): optionally absent
MGCLGWTVLPGTWKFTVTWSDGQTSTGQVHFQPRTPTTLQVHFRGRSSDGRPFNGKGHVTCKTPTTFDVNVTQSDGATSTGKITMKSPTTIDVTFTIEDGQTATGQMHRQS

SEQ ID NO: 3 - protein, 111 aa, Artificial Sequence
  Synthetic
  MISC_FEATURE (1)..(1): optionally absent
MGSALAQQLPGTWKMDVTSEDGVRTTGQMHIQPKTPTTMDVTLTGTHADGKPFTGQGKITVKTPTTVDITVTYEDGSTATGQLTVDSPTQFKFDMTASDGTRFTGTVQRQS

SEQ ID NO: 4 - protein, 110 aa, Artificial Sequence
  Synthetic
  MISC_FEATURE (1)..(1): optionally absent
MCRAASLLPGTWQVTMTNEDGQTSQGQMHFQPRSPYTLDVKAQGTMSDGRPIQGKGKVTCKTPDTMDVDITYSDGKQVQGQVTLDSPTQFKFDVTTSDGSKVTGTLQRQE

SEQ ID NO: 5 - protein, 111 aa, Artificial Sequence
  Synthetic
  MISC_FEATURE (1)..(1): optionally absent
MGTAAVQFLPGTWKFDVTAEDGSQFKGKVHIQPDSPDTVKVTFNGTQSDGKPATGQGTLTMTSPETVKLQVTYEDGKTFTGYMTLRTPTQFQLDAKANDGTKSTGYMRRTE

SEQ ID NO: 6 - protein, 111 aa, Artificial Sequence
  Synthetic
  MISC_FEATURE (1)..(1): optionally absent
MGPAAAQVFPGTWDFQFTAEDGSTFRGKVTFQPRTPTTLDVTMKGTQSDGRPLTGKGKVHVESPTTVQINVTYSDGRTIQGKLTVKTPTTVDVDARFSDGTKSTGKVRRTS

SEQ ID NO: 7 - protein, 111 aa, Artificial Sequence
  Synthetic
  MISC_FEATURE (1)..(1): optionally absent
MGPEAVNILPGDWDVQLHSEDGSTFRGTLRVQPKTPTTLDVTMQGTVSDGRPSDGQGQVHVDSPHDVKITMTMSDGSTATGTLKLHSPTTFQVTLTYADGFTAQGRFTRDG

SEQ ID NO: 8 - protein, 111 aa, Artificial Sequence
  Synthetic
  MISC_FEATURE (1)..(1): optionally absent
MGRAAVQFLPGTWNITSTYEDGTTMQGTVHVTPRSPETFDITVQGQASDGKPMRGQGKVTVQSPHQVQVNLTSEDGTQAQGVFQVDSPTRVKVDLTASDGVRLTGTLQRTS

SEQ ID NO: 9 - protein, 111 aa, Artificial Sequence
  Synthetic
  MISC_FEATURE (1)..(1): optionally absent
MGPCHALELPGTWKFDVTAEDGSQFTGKVTVQPDSPDTVKITFNGTQSDGKPATGQGTLTCTSPETVKITVTYSDGKTFTGYVTLRTPTQFQVDATANDGTKSTGYMRRTE

SEQ ID NO: 10 - protein, 111 aa, Artificial Sequence
  Synthetic
  MISC_FEATURE (1)..(1): optionally absent
MGAEVAQVLPGKWQVHMTNEDGTTSTGTMTVQPRSPYTFDVKFKGTMSDGRPITGNGKVTMKTPDTLDVDLTYSDGKKVTGKVTMRSPTQLDWDLTTSDGSKVTGTSKRQE

SEQ ID NO: 11 - protein, 111 aa, Artificial Sequence
  Synthetic
  MISC_FEATURE (1)..(1): optionally absent
MGPEAVNMLPGDWDIQLTSEDGSQFRGTFRVRPTTPTTVQVTMRGTVSDGRPSQGQGYVTVDSPTDMQVKMTMSDGSQAQGTIKLDSPTTLKIKLTYADGFTAQGTFTRDG

SEQ ID NO: 12 - protein, 111 aa, Artificial Sequence
  Synthetic
  MISC_FEATURE (1)..(1): optionally absent
MGQEVAQVLPGDWQVHMTNEDGQTSTGTVTFQPRSPYTFDVKFKGTMSDGRPITGKGKMTMKTPDTMDIDVTYSDGKKVTGKVTMKSPTQLDWDLTTSDGSKVTGTSHRQE

SEQ ID NO: 13 - protein, 111 aa, Artificial Sequence
  Synthetic
  MISC_FEATURE (1)..(1): optionally absent
MGPEAINVLPGDWDVTFHSEDGSTFRGTVRMQPRTPDTVQVKMTGTVSDGRPSTGTGYVHVDSPHDVKFQMTMSDGSTATGTLKMDSPTQFTVRLTYADGFTAQGHLQRDG

SEQ ID NO: 14 - protein, 111 aa, Artificial Sequence
  Synthetic
  MISC_FEATURE (1)..(1): optionally absent
MGADAQSVLPGTWDINVTFSDGSTFTGTLHFTPRTPTTFDVTLKGHSSDGQPATGQGKVTLKTPTTLDIDIQNSDGSKSTGQITMDTPYDLKFTAKLSDGVTFTGTLKRRE

SEQ ID NO: 15 - protein, 111 aa, Artificial Sequence
  Synthetic
  MISC_FEATURE (1)..(1): optionally absent
MGTSCSSGAPGNWDLQMTSSDGSQSRGTITMKPQTPDQLQVTVKGHFSDGKPFKGTGYVQVTTPTQLTVHLTYSDGRQATGQFNCDTPTDFKVTFTFSDGSTAQGTVKRTE

SEQ ID NO: 16 - protein, 111 aa, Artificial Sequence
  Synthetic
  MISC_FEATURE (1)..(1): optionally absent
MGCDARTVLPGTWDINVTFSDGSTFTGTLHFTPRTPTTFDVTLKGHSSDGQPATGQGKVTLKTPTTLDIDIQNSDGSKSTGQITCDTPYDLKFTAKLSDGVTFTGTLKRRE

SEQ ID NO: 17 - protein, 111 aa, Artificial Sequence
  Synthetic
  MISC_FEATURE (1)..(1): optionally absent
MGQLTCQNLPGKWKFNTTAEDGVTITGTLQVQPRTPTTFDVNLKGHQSDGRPTKGNGKMTCKTPDQVDSRFQLSDGRTFQGKLQVDSPDTLTVNWTMQDGTTQTGQLTRQE

SEQ ID NO: 18 - protein, 111 aa, Artificial Sequence
  Synthetic
  MISC_FEATURE (1)..(1): optionally absent
MGALAQEAAPGTWDVTMTSSDGSQSQGQFHMQPTTPTRFTVTVRGHFSDGKPFKGQGYVDVETPTTLRINVTYSDGSQATGKLQFDTPTDVKVTLTFSDGSTAQGTMKRTE

SEQ ID NO: 19 - protein, 111 aa, Artificial Sequence
  Synthetic
  MISC_FEATURE (1)..(1): optionally absent
MGADVQQVLPGTWDLRVTQEDGTTTQGKVTVKPETPTTLRFTSKGTMSDGKPFHGQGHFTVKSPTTVQIQQTASDGKTATGSLTMKTPTTLDVTMTNQDGTTAQGKMTRKS

SEQ ID NO: 20 - protein, 111 aa, Artificial Sequence
  Synthetic
  MISC_FEATURE (1)..(1): optionally absent
MGPCSTNVLPGTWKFTVTWSDGQTSTGQVHFQPRTPTTLQVHFRGRSSDGRPFNGKGHVTMKTPTTFDVNVTQSDGATSTGKITCKSPTTIDVTFTIEDGQTATGQMHRQS

SEQ ID NO: 21 - protein, 111 aa, Artificial Sequence
  Synthetic
  MISC_FEATURE (1)..(1): optionally absent
MGCDQWRILPGTWDINVTFSDGSTFTGTLHFTPRTPTTFDVTLKGHSSDGQPATGQGKVTLKTCTTLDIDIQNSDGSKSTGQITMDTPYDLKFTAKLSDGVTFTGTLKRRE

SEQ ID NO: 22 - protein, 111 aa, Artificial Sequence
  Synthetic
  MISC_FEATURE (1)..(1): optionally absent
MGKDAANVLPGKWKFNTTAEDGVTITGTLQVQPRTPTTFDVNLKGHQSDGRPTKGNGKMTMKTPDQVDSRFQLSDGRTFQGKLQVDSPDTLTVNWTMQDGTTQTGQLTRQE

SEQ ID NO: 23 - protein, 111 aa, Artificial Sequence
  Synthetic
  MISC_FEATURE (1)..(1): optionally absent
MGSALAQQLPGTWKVDVTSEDGVRTTGQVHFQPRTPTTMDVTLTGTHADGKPFTGQGKVTITTPTTVKVTVTYEDGSTATGQFTVDSPTQLKFDMTASDGTRFTGTVQRQS

SEQ ID NO: 24 - protein, 110 aa, Artificial Sequence; identical to SEQ ID NO: 4
  Synthetic
  MISC_FEATURE (1)..(1): optionally absent

SEQ ID NO: 25 - protein, 112 aa, Artificial Sequence
  Synthetic
  MISC_FEATURE (1)..(1): optionally absent
MCRAASLLPGTWQVTMTNEDGQTSQGQMHFQPRSPYTLDVKAQGTISDGRPISGKGKVTCKTPDTMDVDITYPSLGNMKVQGQVTLDSPTQFKFDVTTSDGSKVTGTLQRQE

SEQ ID NO: 26 - protein, 112 aa, Artificial Sequence
  Synthetic
  MISC_FEATURE (1)..(1): optionally absent
MSRAASLLPGTWQVTMTNEDGQTSQGQMHFQPRSPYTLDVVAQGTISDGRPISGYGKVTVKTPDTMDVDITYPSLGNMKVQGQITLDSPTQFKFDATTSDGSKLTGTLQRQE

SEQ ID NO: 27 - protein, 112 aa, Artificial Sequence
  Synthetic
  MISC_FEATURE (1)..(1): optionally absent
MNRAAQLLPGTWQVTMTNEDGQTSQGQMHFQPRSPYTLDIVAQGTISDGRPISGYGKVTVKTPDTMHVNITYPSLGNIKVQGQITLDSPTQFTWNSTTSDGKKLTGTLQRQE

SEQ ID NO: 28 - protein, 112 aa, Artificial Sequence
  Synthetic
  MISC_FEATURE (1)..(1): optionally absent
MNRAYRMLPGTWQVTMTNEDGQTSQGQMHIQPRSPYTLDVVAQGTISDGRPISGYGKVTVKTPDTLQVHITYPSLGNIKVQGQITLDSPTQFTFNATTSDGKNLTGTLQRQE

SEQ ID NO: 29 - protein, 112 aa, Artificial Sequence
  Synthetic
  MISC_FEATURE (1)..(1): optionally absent
MNRAASLLPGTWQVTMTNEDGQTSQGQMHIQPRSPYTLDVVAQGTISDGRPISGYGKVTVKTPDTMQVHITYPSLGNIKVQGQITLDSPTQFTFNATTSDGKNLTGTLQRQE

SEQ ID NO: 30 - protein, 112 aa, Artificial Sequence
  Synthetic
  MISC_FEATURE (1)..(1): optionally absent
MNRAAANLPGTWQVTMTNEDGQTSQGQMHFQPRSPYTLDVVAQGTISDGRPISGYGKVTVKTPDTMNVDITYPSLGNIKVQGQITLDSPTQFTFNATTSDGKKLTGTIQRQE

SEQ ID NO: 31 - protein, 112 aa, Artificial Sequence
  Synthetic
  MISC_FEATURE (1)..(1): optionally absent
MNTAIANLPGTWQVTMTNEDGQTSQGQMHIQPRSPYTADVVAQGTISDGRPISGYGKLTAKTPDTVNVQITYPSLGNINVQGQITNDSPTQAHFNATTSDGKKLTGTMQRQE

SEQ ID NO: 32 - protein, 112 aa, Artificial Sequence
  Synthetic
  MISC_FEATURE (1)..(1): optionally absent
MSRAAQLLPGTWQVTMTNEDGQTSQGQMHFQPRSPYTLDIVAQGTISDGRPITGYGKVTVKTDDTLHVNITYPSLGNIKVQGQITMDSPTQATWNSTTSDGKKLTGTLQRQE

SEQ ID NO: 33 - protein, 112 aa, Artificial Sequence
  Synthetic
  MISC_FEATURE (1)..(1): optionally absent
MSRAAQLLPGTWQTTMTNEDGQTSQGQMHFQPRSPYTLDVVAQGTISDGRPISGYGKVTVKTDDTLDVDITYPSLGNIKVQGQVTMDSPTQFKFDATTSDGKKLTGTLQRQE

SEQ ID NO: 34 - protein, 112 aa, Artificial Sequence
  Synthetic
  MISC_FEATURE (1)..(1): optionally absent
MSRAAQLLPGTWQSTMTNEDGQTSQGQMHFQPRSPYTLDVVAQGTISDGRPISGYGKVTVKTDDTLDVDITYPSLGNIKVQGQVTMDSPTQFKFDATTSDGKKLTGTLQRQE

SEQ ID NO: 35 - protein, 112 aa, Artificial Sequence
  Synthetic
  MISC_FEATURE (1)..(1): optionally absent
MSRAAQLLPGTWQVTMTNEDGQTTQGQMHFQPRSPYTLDVVAQGTISDGRPISGYGKVTVKTDDTLDVDITYPSLGNIKVQGQVTMDSPTQFKFDATTSDGKKLTGTLQRQE

SEQ ID NO: 36 - protein, 112 aa, Artificial Sequence
  Synthetic
  MISC_FEATURE (1)..(1): optionally absent
MSRAAQLLPGTWQSTMTNEDGQTTQGQMHFQPRSPYTLDVVAQGTISDGRPISGYGKVTVKTDDTLDVDITYPSLGNIKVQGQVTMDSPTQFKFDATTSDGKKLTGTLQRQE

SEQ ID NO: 37 - protein, 112 aa, Artificial Sequence
  Synthetic
  MISC_FEATURE (1)..(1): optionally absent
MSRAAQLLPGTWQVTMTNEDGQTSQGQWHFQPRSPYTLDIVAQGTISDGRPITGYGKATVKTDDTLHANLTYPSLGNIKAQGQITYDSPTQFTWNSTTSDGKKLTGTLQRQE

SEQ ID NO: 38 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPISGYGKVTVKTPDTLDVDITYPSLGNIKAQGQITMDSPTQFKFDATTKGAGNFTGRLTGTLQRQE

SEQ ID NO: 39 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVSQGTISDGRPISGYGKVTVKTPDTLDVDITYPSLGNIKAQGQITMDSPTQFKFDATTKGAGNFTGRLTGTLQRQE

SEQ ID NO: 40 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRTIVGYGKVTVKTPDTLDVDITYPSLGNIKAQGQITMDSPTQFKFDATTKGAGNFTGRLTGTLQRQE

SEQ ID NO: 41 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGAGNFTGRLTGTLQRQE

SEQ ID NO: 42 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGDDGFQGRLTGTLQRQE

SEQ ID NO: 43 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGEDGFQGRLTGTLQRQE

SEQ ID NO: 44 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGENDFVGRLTGTLQRQE

SEQ ID NO: 45 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGENDFQGRLTGTLQRQE

SEQ ID NO: 46 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGADGWHGRLTGTLQRQE

SEQ ID NO: 47 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGEDGWVGRLTGTLQRQE

SEQ ID NO: 48 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGADGWDGRLTGTLQRQE

SEQ ID NO: 49 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGEDDYVGRLTGTLQRQE

SEQ ID NO: 50 - protein, 116 aa, Artificial Sequence
  Synthetic
  MISC_FEATURE (1)..(1): optionally absent
MSRAAQLLPGTWQVTMTNEDGQTSQGQWHFQPRSPYTMDVVAQGTISDGRPIVGYGKATVKTPDTLDVDITYPSLGNIKAQGQITMDSPTQFKWDATTKGAGNFTGRLTGTLQRQE

SEQ ID NO: 51 - protein, 116 aa, Artificial Sequence
  Synthetic
  MISC_FEATURE (1)..(1): optionally absent
MSRAAQLLPGTWQVTMTNEDGQTSQGQWHFQPRSPYTMDVVAQGTISDGRPIVGYGKATVKTPDTLDVDLTYPSLGNIKAQGQITMDSPTQFKWDATTKGAGNFTGRLTGTLQRQE

SEQ ID NO: 52 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDVDITYPSLGNLKAQGQITMDSPTQFKFDATTKGAGNFTGRLTGTLQRQE

SEQ ID NO: 53 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDVDITYPSLGNIKAQGQITMDSPTQFKWDATTKGAGNFTGRLTGTLQRQE

SEQ ID NO: 54 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDVDITYPSLGNIKAQGQITMDSPTQFKFDSTTKGAGNFTGRLTGTLQRQE

SEQ ID NO: 55 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKFDSTTKGAGNFTGRLTGTLQRQE

SEQ ID NO: 56 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDADITYPSLGNIKAQGQITMDSPTQFKFDATTKGAGNFTGRLTGTLQRQE

SEQ ID NO: 57 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDADLTYPSLGNLKAQGQITMDSPTQFKFDATTKGAGNFTGRLTGTLQRQE

SEQ ID NO: 58 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDVDITYPSLGNIKAQGQITMDSPTQFKFDATTKGENDFHGRLTGTLQRQE

SEQ ID NO: 59 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKFDATTKGAGNFTGRLTGTLQRQE

SEQ ID NO: 60 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDVDITYPSLGNIKAQGQITMDSPTQFKWDATTKGENDFHGRLTGTLQRQE

SEQ ID NO: 61 - protein, 116 aa, Artificial Sequence
  Synthetic
  MISC_FEATURE (1)..(1): optionally absent
MSRAAQLLPGTWQLTMTNEDGQTSQGQWHFQPRSPYTMDIVAQGTISDGRPIVGYGKATVKTPDTLDVDITYPSLGNIKAQGQITMDSPTQFKWDATTKGENDFHGRLTGTLQRQE

SEQ ID NO: 62 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDVDITYPSLGNIKAQGQITMDSPTQFKWDATTKGENYFHGRLTGTLQRQE

SEQ ID NO: 63 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDLDVTYPSLGNIKAQGQITMDSPTQFKWDATTKGENDFHGRLTGTLQRQE

SEQ ID NO: 64 - protein, 116 aa, Artificial Sequence
  Synthetic
  MISC_FEATURE (1)..(1): optionally absent
MSRAAQLLPGTWQATMTNEDGQTSQGQWHFQPRSPYTMDIVAQGTISDGRPIVGYGKATVKTPDTLDVDVTYPSLGNIKAQGQITMDSPTQFKWDATTKGENDFHGRLTGTLQRQE

SEQ ID NO: 65 - protein, 116 aa, Artificial Sequence
  Synthetic
  MISC_FEATURE (1)..(1): optionally absent
MSRAAQLLPGTWQITMTNEDGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 66 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 67 - protein, 116 aa, Artificial Sequence
  Synthetic
  MISC_FEATURE (1)..(1): optionally absent
MSRAAQLLPGTWQVTFTNEDGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 68 - protein, 116 aa, Artificial Sequence
  Synthetic
  MISC_FEATURE (1)..(1): optionally absent
MSRAAQLLPGTWQGTFTNEDGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 69 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 70 - protein, 121 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAQFKQNDNPRYHGSTNILTGTLQRQE

SEQ ID NO: 71 - protein, 121 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVKSNDSPSLSGNTKILTGTLQRQE

SEQ ID NO: 72 - protein, 121 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAQFKKDDDPRYHGSTHILTGTLQRQE

SEQ ID NO: 73 - protein, 121 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAEYKKNDDPRLKGSKSILTGTLQRQE

SEQ ID NO: 74 - protein, 121 aa, Artificial Sequence
  Synthetic
  MISC_FEATURE (1)..(1): optionally absent
MSRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHYKTNDYPSLNGSKSILTGTLQRQE

SEQ ID NO: 75 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDVDITYPSLGNLKAQGQITMDSPTQFKFDATTKGENDFHGRLTGTLQRQE

SEQ ID NO: 76 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDIDITYPSLGNIKAQGQITMDSPTQFKWDATTKGENDFHGRLTGTLQRQE

SEQ ID NO: 77 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKWDATTKGENDFHGRLTGTLQRQE

SEQ ID NO: 78 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDADLTYPSLGNLKAQGQITMDSPTQFKFDATTKGENDFHGRLTGTLQRQE

SEQ ID NO: 79 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKFDATTKGENDFHGRLTGTLQRQE

SEQ ID NO: 80 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDADITYPSLGNIKAQGQITMDSPTQFKFDATTKGENDFHGRLTGTLQRQE

SEQ ID NO: 81 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDIDITYPSLGNIKAQGQITMDSPTQFKWDATTKGADGWHGRLTGTLQRQE

SEQ ID NO: 82 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDADITYPSLGNIKAQGQITMDSPTQFKFDATTKGADGWHGRLTGTLQRQE

SEQ ID NO: 83 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKATVKTPDTLDVDITYPSLGNLKAQGQITMDSPTQFKFDATTKGADGWHGRLTGTLQRQE

SEQ ID NO: 84 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKFDATTKGADGWHGRLTGTLQRQE

SEQ ID NO: 85 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDVDITYPSLGNIKAQGQITMDSPTQFKWDATTKGADGWHGRLTGTLQRQE

SEQ ID NO: 86 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDIDITYPSLGNIKAQGQITMDSPTQFKFDATTKGADGWHGRLTGTLQRQE

SEQ ID NO: 87 - protein, 116 aa, Artificial Sequence
  Synthetic
  MISC_FEATURE (1)..(1): optionally absent
MSRAAQLLPGTWQVTMTNEDGQTSQGQMHFQPRSPYTMDVVAQGTISDGRPIVGYGKVTVKTPDTLDADLTYPSLGNLKAQGQITMDSPTQFKFDATTKGADGWHGRLTGTLQRQE

SEQ ID NO: 88 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITWPSLGNIKGQGQITMDSPTQFKWDGTTKGENDFHGRLTGTLQRQE

SEQ ID NO: 89 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITWPSLGNIKAQGQITMDSPTQFKWDGTTKGENDFHGRLTGTLQRQE

SEQ ID NO: 90 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITWPSLGNIKAQGQITMDSPTQFKWDATTKGENDFHGRLTGTLQRQE

SEQ ID NO: 91 - protein, 116 aa, Artificial Sequence
  Synthetic
  MISC_FEATURE (1)..(1): optionally absent
MSRAAQLLPGTWQVTMTNEDGQTSQGQWHFQPRSPYTMDIVAQGTLSDGRPIVGYGKATVKTPDTLDIDITWPSLGNIKAQGQITMDSPTQFKWDATTKGENDFHGRLTGTLQRQE

SEQ ID NO: 92 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITYPSLGNIKGQGQITMDSPTQFKWDGTTKGENDFHGRLTGTLQRQE

SEQ ID NO: 93 - protein, 116 aa, Artificial Sequence
  Synthetic
  MISC_FEATURE (1)..(1): optionally absent
MSRAAQLLPGTWQATFTNEDGQTSQGQWHFQPRSPYTMDIVAQGTISDGRPIVGYGKATVKTPDTLDIDITWPSLGNIKVQGQITMDSPTQFKWDGTTKGENDFHGRLTGTLQRQE

SEQ ID NO: 94 - protein, 116 aa, Artificial Sequence
  Synthetic
  MISC_FEATURE (1)..(1): optionally absent
MSRAAQLLPGTWQVTMTNEDGQTSQGQMHFQPRSPYTMDIVAQGTISDGRPIVGYGKATVKTPDTLDIDITYPSLGNIKAQGQITMDSPTQFKFDATTKGENDFHGRLTGTLQRQE

SEQ ID NO: 95 - protein, 125 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQTKILTGTLQRQE

SEQ ID NO: 96 - protein, 125 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQPHILTGTLQRQE

SEQ ID NO: 97 - protein, 125 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQPHILTGTLQRQE

SEQ ID NO: 98 - protein, 124 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQPRLTGTLQRQE

SEQ ID NO: 99 - protein, 124 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQPRLTGTLQRQE

SEQ ID NO: 100 - protein, 125 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGDGYISAAEAAAQTKILTGTLQRQE

SEQ ID NO: 101 - protein, 125 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHYKGDKDGDGYISAAEAAAQILTGTLQRQE

SEQ ID NO: 102 - protein, 125 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAEYKGDKDGDGYISAAEAAAQILTGTLQRQE

SEQ ID NO: 103 - protein, 125 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAEYKGDKDGDGYISAAEAAAQILTGTLQRQE

SEQ ID NO: 104 - protein, 125 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHYKGDKDGDGYISAAEAAAQGLTGTLQRQE

SEQ ID NO: 105 - protein, 125 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHYKGDKDGDGYISAAEAAAQGLTGTLQRQE

SEQ ID NO: 106 - protein, 125 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHYKGDKDGDGYISAAEAAAQILTGTLQRQE

SEQ ID NO: 107 - protein, 160 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNYEEFVQMPRLTGTLQRQE

SEQ ID NO: 108 - protein, 160 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAEEFVQMPRLTGTLQRQE

SEQ ID NO: 109 - protein, 160 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAEEFVQMPRLTGTLQRQE

SEQ ID NO: 110 - protein, 161 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAAEAAQMTKILTGTLQRQE

SEQ ID NO: 111 - protein, 161 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAAEAAQMTKILTGTLQRQE

SEQ ID NO: 112 - protein, 163 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDATTKGDKDGNGYISAAELRHVMTNLGEKLTDEEVDEMIREADIDGDGQVNAAEAAAMTKILTGTLQRQE

SEQ ID NO: 113 - protein, 163 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDATTKGDKDGNGYISAAELRHVMTNLGEKLTDEEVDEMIREADIDGDGQVNAAEAAAMTKILTGTLQRQE

SEQ ID NO: 114 - protein, 162 aa, Artificial Sequence
  Synthetic
SRAAQLLPGTWQVTMTNEDGQTSQGQWHFRPRSPYTMDIVAQGTISDGRPIVGYGKATVKTPDTLDIDITYPSLGNIKAQGQITMDSPTQFKWDAHVKGDKDGNGYISAAELRHVMTNLGEKLTDEEVDEMIREADIDGDGQVNYEEFVQMPRLTGTLQRQE

SEQ ID NO: 115 - protein, 162 aa, Artificial Sequence
  Synthetic
SRAAQLLPGTWQATFTNEDGQTSQGQWHFRPRSPYTMDIVAQGTISDGRPIVGYGKATVKTPDTLDIDITYPSLGNIKAQGQITMDSPTQFKWDAHVKGDKDGNGYISAAELRHVMTNLGEKLTDEEVDEMIREADIDGDGQVNYEEFVQMPRLTGTLQRQE

SEQ ID NO: 116 - protein, 160 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAAEAAAMPRLTGTLQRQE

SEQ ID NO: 117 - protein, 160 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNAAEAAAMPRLTGTLQRQE

SEQ ID NO: 118 - protein, 160 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVDKDGNGYISAAELRHVMTNLGEKLTDEEVDEMIREADIDGDGQVNYEEFVQMPRLTGTLQRQE

SEQ ID NO: 119 - protein, 236 aa, Artificial Sequence
  Synthetic
SRAAQLLPGTWQVTMTNEDGQTSQGQWHFQPRSPYTMDIVAQGTISDGRPIVGYGKATVKTPDTLDIDITYPSLGNIKAQGQITMDSPTQFKWDAHVKGDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVQMPHILTGTLQRQE

SEQ ID NO: 120 - protein, 236 aa, Artificial Sequence
  Synthetic
SRAAQLLPGTWQATFTNEDGQTSQGQWHFQPRSPYTMDIVAQGTISDGRPIVGYGKATVKTPDTLDIDITYPSLGNIKAQGQITMDSPTQFKWDAHVKGDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVQMPHILTGTLQRQE

SEQ ID NO: 121 - protein, 7 aa, Artificial Sequence
  Synthetic
  MISC_FEATURE (3)..(3): X is A, I, or Y
  MISC_FEATURE (4)..(4): X is R, S, Q, or A
RAXXLLP

SEQ ID NO: 122 - protein, 9 aa, Artificial Sequence
  Synthetic
  MISC_FEATURE (2)..(2): X is T, K, N, or D
  MISC_FEATURE (7)..(7): X is M or F
GXWQZTXTN

SEQ ID NO: 123 - protein, 11 aa, Artificial Sequence
  Synthetic
QTSQGQMHFQP

SEQ ID NO: 124 - protein, 9 aa, Artificial Sequence
  Synthetic
  MISC_FEATURE (2)..(2): X is L, A, or M
  MISC_FEATURE (4)..(4): X is I or V
  MISC_FEATURE (5)..(5): X is K or V
  MISC_FEATURE (6)..(6): X is A or S
  MISC_FEATURE (9)..(9): X is I or M
TXDXXXGTX

SEQ ID NO: 125 - protein, 11 aa, Artificial Sequence
  Synthetic
  MISC_FEATURE (4)..(4): X is Q, S, T, or V
  MISC_FEATURE (6)..(6): X is Y or K
  MISC_FEATURE (9)..(9): X is L, V, or A
  MISC_FEATURE (11)..(11): X is C, V, or A
RPIXGXGKXTX

SEQ ID NO: 126 - protein, 8 aa, Artificial Sequence
  Synthetic
  MISC_FEATURE (2)..(2): X is M, L, or V
  MISC_FEATURE (3)..(3): X is D, H, Q, or N
  MISC_FEATURE (4)..(4): X is V, A, L, or I
  MISC_FEATURE (5)..(5): X is D, N, H, or Q
  MISC_FEATURE (6)..(6): X is I, L, or V
  MISC_FEATURE (8)..(8): X is Y or W
TXXXXXTX

SEQ ID NO: 127 - protein, 5 aa, Artificial Sequence
  Synthetic
PSLGN

SEQ ID NO: 128 - protein, 9 aa, Artificial Sequence
  Synthetic
  MISC_FEATURE (1)..(1): X is K, M, I, or L
  MISC_FEATURE (2)..(2): X is K or Q
  MISC_FEATURE (3)..(3): X is V, A, or G
  MISC_FEATURE (7)..(7): X is V or I
  MISC_FEATURE (9)..(9): X is M, L or Y
XXXQGQXTX

SEQ ID NO: 129 - protein, 8 aa, Artificial Sequence
  Synthetic
  MISC_FEATURE (2)..(2): X is F or A
  MISC_FEATURE (3)..(3): X is K, T or H
  MISC_FEATURE (4)..(4): X is F or W
  MISC_FEATURE (5)..(5): X is D or N
  MISC_FEATURE (6)..(6): X is V, A, S, or G
  MISC_FEATURE (7)..(7): X is T, Q, H or E
  MISC_FEATURE (8)..(8): X is T, F, V or Y
QXXXXXXX

SEQ ID NO: 130 - protein, 5 aa, Artificial Sequence
  Synthetic
  MISC_FEATURE (2)..(2): X is G, Q, K, or T
  MISC_FEATURE (3)..(3): X is A, D, E, or N
  MISC_FEATURE (4)..(4): X is D, G, or N
  MISC_FEATURE (5)..(5): X is D, G, Y, S or N
KXXXX

SEQ ID NO: 131 - protein, 5 aa, Artificial Sequence
  Synthetic
  MISC_FEATURE (3)..(3): X is A, D, or E
  MISC_FEATURE (4)..(4): X is G, D, or N
  MISC_FEATURE (5)..(5): X is G, D, Y or N
KGXXX

SEQ ID NO: 132 - protein, 11 aa, Artificial Sequence
  Synthetic
  MISC_FEATURE (1)..(1): X is S, K, N, H or optionally absent
  MISC_FEATURE (2)..(2): X is K, N, I or R
  MISC_FEATURE (3)..(3): X is V or L
  MISC_FEATURE (7)..(7): X is I, M or L
XXXTGTXQRQE

SEQ ID NO: 133 - protein, 116 aa, Artificial Sequence; identical to SEQ ID NO: 69
  Synthetic

SEQ ID NO: 134 - protein, 7 aa, Artificial Sequence
  Synthetic
RAAQLLP

SEQ ID NO: 135 - protein, 9 aa, Artificial Sequence
  Synthetic
  MISC_FEATURE (5)..(5): X is V, L, A, or I
  MISC_FEATURE (7)..(7): X is M or F
GTWQXTXTN

SEQ ID NO: 136 - protein, 10 aa, Artificial Sequence
  Synthetic
TMDIVAQGTI

SEQ ID NO: 137 - protein, 11 aa, Artificial Sequence
  Synthetic
RPIVGYGKATV

SEQ ID NO: 138 - protein, 8 aa, Artificial Sequence
  Synthetic
TLDIDITY

SEQ ID NO: 139 - protein, 9 aa, Artificial Sequence
  Synthetic
IKAQGQITM

SEQ ID NO: 140 - protein, 8 aa, Artificial Sequence
  Synthetic
QFKFDATT

SEQ ID NO: 141 - protein, 8 aa, Artificial Sequence
  Synthetic
KGENDFHG

SEQ ID NO: 142 - protein, 8 aa, Artificial Sequence
  Synthetic
KGADGWHG

SEQ ID NO: 143 - protein, 8 aa, Artificial Sequence
  Synthetic
KGAGNFTG

SEQ ID NO: 144 - protein, 10 aa, Artificial Sequence
  Synthetic
RLTGTLQRQE